(19) United States
(12) Patent Application Publication
(10) Pub. No.: US 2025/0115935 A1
(43) Pub. Date: Apr. 10, 2025

(54) ENGINEERED MULTIPARTITE TRANSCRIPTIONAL EFFECTORS SOURCED FROM HUMAN PROTEIN DOMAINS

(71) Applicant: William Marsh Rice University, Houston, TX (US)

(72) Inventors: Isaac HILTON, Houston, TX (US); Barun MAHATA, Houston, TX (US); Jacob GOELL, Houston, TX (US)

(73) Assignee: William Marsh Rice University, Houston, TX (US)

(21) Appl. No.: 18/834,826

(22) PCT Filed: Jan. 31, 2023
(86) PCT No.: PCT/US2023/061620

§ 371 Date: Jul. 31, 2024

Related U.S. Application Data
(60) Provisional application No. 63/305,040, filed on Jan. 31, 2022.

(51) Int. Cl. C12N 15/90; C12N 9/22; C12N 15/11

(52) U.S. Cl. CPC C12N 15/907; C12N 9/22; C12N 15/11; C12N 2310/20; C12N 2830/15

(57) ABSTRACT

The present disclosure is directed to designed fusion proteins derived from MTFs with strong potency to modulate transcription and designated these recombinant fusion proteins MSN and NMS. These powerful transactivators potently activate transcription from endogenous loci when recruited through CRISPR-dCas9, Zinc Finger, or TALE system proteins. This technology permits upregulation of gene expression in targeted manner devoid of viral transcription activation domains and is amenable to high-throughput screening. These synthetic transcription activators interact with all programable DNA binding proteins tested and have exhibited applicability in vitro for efficient lineage conversion.

### PRIORITY CLAIM

This application claims benefit of priority to U.S. Provisional Application Ser. No. 63/305,040, filed Jan. 31, 2022, the entire contents of which are hereby incorporated by reference.

### STATEMENT REGARDING FEDERAL FUNDING

This invention was made with government support under Grant Nos. R35GM143532 and R21EB030772 awarded by the National Institutes of Health. The government has certain rights in the invention.

### SEQUENCE LISTING

This application contains a Sequence Listing XML, which has been submitted electronically and is hereby incorporated by reference in its entirety. Said XML Sequence Listing, created on Jan. 30, 2023, is named RICEP0092WO.xml and is 6,945 bytes in size.

### BACKGROUND

### 1. Field of the Disclosure

The present disclosure relates generally to the fields of molecular biology and gene expression. More particular, the disclosure relates to multipartite transcriptional effectors and uses thereof.

### 2. Background

Nuclease deactivated CRISPR-Cas (dCas) systems can be used to modulate transcription in cells and organisms¹⁻⁸. For CRISPR-based activation (CRISPRa) approaches, transcriptional activators can be recruited to genomic regulatory elements using direct fusions to dCas proteins⁹⁻¹³, antibody-mediated recruitment¹⁴, or using engineered gRNA architectures^{15, 16}. High levels of CRISPRa-driven transactivation have been achieved by shuffling¹⁷, reengineering¹⁸, or combining^{9, 19, 20} transactivation domains (TADs) and/or chromatin modifiers. However, many of the transactivation components used in these CRISPRa systems have coding sizes that are restrictive for applications such as viral vector-based delivery. Moreover, most of the transactivation modules that display high potencies harbor components derived from viral pathogens and are poorly tolerated in clinically important cell types, which could hamper biomedical or in vivo use. Finally, there is an untapped repertoire of thousands of human transcription factors (TFs) and chromatin that has yet to be systematically tested and optimized as programmable modifiers²¹⁻²⁴ transactivation components. This diverse repertoire of human protein building blocks could be used to reduce the size of transactivation components, obviate the use of viral TFs, and possibly permit cell and/or pathway specific transactivation.

Mechanosensitive transcription factors (MTFs) modulate transcription in response to mechanical cues and/or external ligands^{25, 26}. When stimulated, MTFs are shuttled into the nucleus where they can rapidly transactivate target genes by engaging key nuclear factors including RNA polymerase II (RNAP) and/or histone modifiers²⁷⁻³⁰. The dynamic shuttling of MTFs can depend upon both the nature and the intensity of stimulation. Mammalian cells encode several classes of MTFs, including serum regulated MTFs (e.g., YAP, TAZ, SRF, MRTF-A and B, and MYOCD)^{26, 31}, cytokine regulated/JAK-STAT family MTFs (e.g., STAT proteins)³², and oxidative stress/antioxidant regulated MTFs (e.g., NRF2)³³; each of which can potently activate transcription when appropriately stimulated.

### SUMMARY

Thus, in accordance with the present disclosure, recombinant transcription activators comprising transcription activation domains from MRTF-A, STAT1 and eNRF2 are described. The recombinant transcription activators may further comprise a genomic regulatory element targeting domain and/or RNA-binding protein. The RNA-binding protein can be any protein that specifically binds RNA, such as one containing an MCP or PCP domain. Other examples include RNA-binding proteins/domains from PP7, Pumilio or RNA-binding Cas species distinct from the genomic regulatory element. The genomic regulatory element targeting domain may be a Cas protein, such as Cas6, AsdCas12a, SpdCas9, CjdCas9, or SadCas9. The genomic regulatory element targeting domain may also be a TALE DNA binding domain or a zinc finger DNA binding domain. The transcription activation domains may be ordered MRTF-A, STAT1 and eNRF2 in an N- to C-terminal order or may be ordered eNRF2, MRTF-A and STAT1 in an N- to C-terminal order. The transcription activation domains may be directly linked to said genomic regulatory element targeting domain or linked to said genomic regulatory element targeting domain through a linking moiety, such as where the linking moiety is GS or XTEN. The recombinant transcription activator may be about 250-500 or about 290 amino acid residues in length.

Also provided is a recombinant nucleic acid segment encoding a transcription activator comprising transcription activation domains MRTF-A, STAT1 and eNRF2. The nucleic acid may further comprise a nucleic acid segment encoding a genomic regulatory element targeting domain and/or RNA-binding protein. The RNA-binding protein can be any protein that specifically binds RNA, such as one containing an MCP or PCP domain. Other examples include RNA-binding proteins/domains from PP7, Pumilio or RNA-binding Cas species distinct from the genomic regulatory element. The genomic regulatory element targeting domain may be a Cas protein, such as Cas6, AsdCas12a, SpdCas9, CjdCas9, or SadCas9. The genomic regulatory element targeting domain may be a TALE DNA binding domain or a zinc finger DNA binding domain. The transcription activation domains may be ordered MRTF-A, STAT1 and eNRF2 in an N- to C-terminal order or may be ordered eNRF2, MRTF-A and STAT1 in an N- to C-terminal order. The transcription activation domains may be directly linked to said genomic regulatory element targeting domain or linked to said genomic regulatory element targeting domain through a linking moiety, such as where the linking moiety is GS or XTEN. The recombinant transcription activator may be about 750-1500 or about 870 bases in length. The promoter active may be eukaryotic cell is EFS or CMV.

In another embodiment, there is provided an artificial recombinant transcription factor comprising or consisting of at least 2 or at least 3 repeated 9aa TADs generated from MRTF-B and MYOCD or transcription factors. The recombinant transcription factor may be about or less than 300 amino acids in size. The MRTF-B and MYOCD features may be linked by linking moiety, such as the linking moieties GS and/or XTEN.

In still another embodiment, there is provided a method of editing gene expression in a eukaryotic cell comprising transferring into said cell the nucleic acid segment as defined above. The gene regulatory element targeting domain may be a Cas protein, and the method may comprise providing to said cell a guide RNA. The eukaryotic cell may be an isolated cell in culture, derived from a living organism, a human cell, non-human mammalian cell or a fibroblast. The editing may result in one or more of (a) increased gene expression of one or multiple genes, (b) induction of cellular differentiation, (c) induction of cellular de-differentiation. The editing may result in induction of pluripotency/stem cells from a differentiated cell. The editing may result in expression of a native/endogenous gene in a cell deficient in expression of said native gene/endogenous gene. The editing may result in expression of a non-native/exogenous gene such that said cell is protected from or at reduced risk of development of a disease state, disease condition or disorder. The editing system may be delivered via a viral mechanism, such as adeno-associated virus, lentivirus, retrovirus, herpesvirus, baculovirus, or adenovirus or delivered via a non-viral mechanism, such as electroporation, nucleofection, mechanical stress, or liposomal transfer.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIGS. 1a-i. CRISPR-DREAM displays potent activation at human promoters, has high specificity, and is robust across cell types. FIG. 1a. Nuclease inactivated Streptococcus pyogenes dCas9 (dCas9), a gRNA containing two engineered MS2 stem-loops (MS2 SLs) and MS2 binding Cap Protein (MCP)-fused transcriptional effector proteins are schematically depicted. Nuclease-inactivating mutations (D10A and H840A) are indicated by yellow bars with dots above. FIG. 1b. dCas9 and MCP-fusion proteins, including an MCP-mCherry fusion (Control; top), the engineered tripartite MCP-MSN domain fusion (DREAM system; middle), and dCas9-VP64 and the MCP-p65-HSF1 fusion protein (SAM system; bottom) are schematically depicted. FIG. 1c. The expression levels of dCas9 and dCas9-VP64 (top), FLAG tagged MCP-mCherry, FLAG tagged MCP-MSN, FLAG tagged MCP-p65-HSF1 (middle), and β-Tubulin (loading control; bottom) are shown as detected by Western blotting in HEK293T cells 72 hrs post-transfection. FIG. 1d and FIG. 1e. Relative expression levels of endogenous human genes 72 hrs after Control, DREAM, or SAM systems were targeted to their respective promoters using pools of 4 or 3 gRNAs (HBG1 and CD34, respectively; FIG. 1d), or using single gRNAs (ACE2 and HGF, respectively; FIG. 1e) as measured by QPCR. FIG. 1f. Transcriptome wide RNA-seq data generated 72 hrs after the DREAM (top) or SAM (bottom) systems were targeted to the HBG1/HBG2 promoter using 4 pooled gRNAs. mRNAs identified as significantly differentially expressed (fold change >2 or <−2 and FDR <0.05) are shown as red dots in both MA plots. In the top MA plot (CRISPR-DREAM), mRNAs corresponding to HBG1/HBG2 (target genes) are highlighted in light blue. mRNAs encoding components of the MSN tripartite fusion protein (MRTF-A/STAT1/NRF2; red), were also significantly differentially expressed (fold change >2 and FDR <0.05). In the bottom MA plot (SAM system), mRNAs corresponding to HBG1/HBG2 (target genes) are highlighted in light gray. HSF1 mRNA (a component of the p65-HSF1 bipartite fusion protein; red), was also significantly differentially expressed (fold change >2 and FDR <0.05). FIG. 1g. 6 endogenous genes were activated by DREAM or SAM using a pool of gRNAs (1 gRNA/gene) in HEK293T cells. FIGS. 1h and i. OCT4 (FIG. 1h) or HBG1 (FIG. 1i) gene activation by DREAM or SAM systems when corresponding promoters were targeted by 4 gRNAs per promoter in hTERT-MSC or PMBC cells, respectively. All QPCR samples were processed 72 hrs post-transfection and are the result of at least 3 biological replicates. Error bars; SEM. *; P<0.05, **; P<0.01, ***, P<0.001. ns; not significant.

FIGS. 2a-i. CRISPR-DREAM efficiently activates transcription from diverse human regulatory elements. FIG. 2a-c. CRISPR-DREAM and the SAM system activated downstream mRNA expression from OCT4 (FIG. 2a), HBE, HBG, and HBD (FIG. 2b), and SOCS1 (FIG. 2c), when targeted to the OCT4 distal enhancer (DE), HS2 enhancer, or one of two intragenic SOCS1 enhancers, using pools of 3 (OCT4 DE), 4 (HS2), 3 (SOCS1+15 kb), or 2 (SOCS1+50 kb) gRNAs respectively. d. CRISPR-DREAM and the SAM system activated sense eRNA expression when targeted to the NET1 enhancer using 2 gRNAs. FIGS. 2e-f. CRISPR-DREAM and the SAM system bidirectionally activated eRNA expression when targeted to the KLK3 (FIG. 2e) or TFF1 (FIG. 2f) enhancers using pools of 4 or 3 gRNAs, respectively. FIG. 2g and FIG. 2h. CRISPR-DREAM and the SAM system activated the expression of long noncoding RNA when targeted to the CCAT1 (FIG. 2g) or GRASLND (FIG. 2h) promoters using pools of 4 gRNAs, respectively. FIG. 2i. CRISPR-DREAM and the SAM system activated the expression of pre and mature miR-146a when targeted to the miR-146a promoter using a pool of 4 gRNAs. All samples were processed for QPCR 72 hrs post-transfection. Data are the result of at least 4 biological replicates. Error bars; SEM. *; P<0.05, **; P<0.01, ***; P<0.001. ns; not significant.

FIGS. 3a-l. CRISPR-DREAM is portable to orthogonal dCas9 proteins and amenable to miniaturization. FIG. 3a. The SadCas9-DREAM system is schematically depicted, and nuclease-inactivating mutations (D10A and N580A) are indicated by yellow bars with dots above. FIG. 3b. HBG1 (left) or TTN (right) gene activation using the SadCas9-DREAM or SadCas9-SAM systems, when targeted to each corresponding promoter using pools of 4 gRNAs, respectively. FIG. 3c. HBG1 (left) or TTN (right) gene activation using the SadCas9-DREAM or SadCas9-VPR systems, when targeted to each corresponding promoter using pools of 4 MS2-modified (SadCas9-DREAM) or standard gRNAs (SadCas9-VPR), respectively. FIG. 3d. The CjdCas9-DREAM system is schematically depicted, and nuclease-inactivating mutations (D8A and H559A) are indicated by yellow bars with dots above. FIG. 3e. HBG1 (left) or TTN (right) gene activation using the CjdCas9-DREAM or CjdCas9-SAM systems, when targeted to each corresponding promoter using pools of 3 MS2-modified gRNAs, respectively. FIG. 3f. HBG1 (left) or TTN (right) gene activation using the CjdCas9-DREAM or MiniCAFE systems, when targeted to each corresponding promoter using pools of 3 MS2-modified (SadCas9-DREAM) or standard gRNAs (miniCAFE), respectively. FIG. 3g. A 3× 9aa TAD derived from MYOCD and MRTF-B TADs is schematically depicted, GS; glycine-serine linker. FIG. 3h. HBG1 (left) or TTN (right) gene activation when the 3× 9aa TAD was fused to MCP and recruited to each corresponding promoter using dCas9 and a pool of 4 MS2-modified gRNAs, respectively. FIG. 3i. The mini-DREAM system is schematically depicted. MCP-eN3×9 is a fusion protein consisting of MCP, eNRF2, and the 3× 9aa TAD derived from MYOCD and MRTF-B TADs. FIG. 3j. HBG1 (left) or TTN (right) gene activation when either the mini-DREAM or CRISPR-DREAM system was targeted to each corresponding promoter using a pool of 4 MS2-modified gRNAs, respectively. FIG. 3k. The mini-DREAM Compact system is schematically depicted, P2A; self-cleaving peptide. FIG. 31. HIBG1 (left) or TTN (right) gene activation when either the mini-DREAM Compact or mini-DREAM system was targeted to each corresponding promoter using a pool of 4 MS2-modified gRNAs, respectively. All samples were processed for QPCR 72 hrs post-transfection. Data are the result of at least 3 biological replicates. Error bars; SEM. *; P<0.05, **; P<0.01, ***; P<0.001. ns; not significant.

FIGS. 4a-i. The MSN and NMS effector domains are portable to diverse DNA binding platforms and enable superior multiplexing when fused to dCas12a. FIG. 4a. Synthetic transcription activator-like effector (TALE) proteins harboring indicated effector domains were designed to target the human IL1RN promoter. Repeat variable di-residues, RVDs. Relative IL1RN expression (bottom) 72 hrs after indicated TALE fusion protein encoding plasmids were transfected. FIG. 4b. Synthetic zinc finger (ZF) proteins harboring indicated effector domains were designed to target the human ICAM1 promoter. Relative ICAM1 expression (bottom) 72 hrs after indicated ZF fusion protein encoding plasmids were transfected. FIG. 4c. The Type I CRISPR system derived from E. Coli K-12 (Eco-cascade) is schematically depicted along with an effector fused to the Cas6 protein subunit. FIG. 4d. HBG1 gene activation when either the MSN, NMS, or p300 effector domains were fused to Cas6 and the respective engineered Eco-Cascade complexes were targeted to the HBG1 promoter using a single crRNA. FIG. 4e. Multiplexed activation of 6 endogenous genes 72 hrs after co-transfection of Eco-cascade complexes when MSN was fused to Cas6 and targeted using a single crRNA array expression plasmid (1 crRNA/promoter). FIG. 4f. The dCas 12a protein and indicated fusions are schematically depicted along with the G993A DNase-inactivating mutation indicated by a yellow bar with a dot above. FIG. 4g and FIG. 4h. IL1B (FIG. 4g) or TTN (FIG. 4h) gene activation using the indicated dCas12a fusion proteins when targeted to each corresponding promoter using a pool of 2 crRNAs (for IL1B) or a single array encoding 3 crRNAs (TTN), respectively. FIG. 4i. Multiplexed activation of 16 indicated endogenous genes 72 hrs after co-transfection of dCas12a-NMS and a single crRNA array expression plasmid encoding 20 crRNAs. All samples were processed for QPCR 72 hrs post-transfection in HEK293T cells. Data are the result of at least 4 biological replicates. Error bars; SEM. **; P<0.01.

FIGS. 5a-e. dCas9-NMS permits efficient in vitro reprogramming of human fibroblasts. FIG. 5a. Primary human foreskin fibroblasts (HFFs) were nucleofected with plasmids encoding 15 multiplexed gRNAs targeting the OCT4, SOX2, KLF4, c-MYC, and LIN28A promoter and EEA motifs (as in previous reports¹⁷), and either dCas9-NMS (middle row) or dCas9-VP192 (bottom row). HFF morphology was analyzed 8 and 16 days later (white scale bars, 100 μm). FIG. 5b. Relative expression of pluripotency-associated genes OCT4 (left) and SOX2 (right) in representative iPSC colonies (C1 or C2) approximately 40 days after nucleofection of either dCas9-NMS (blue) or dCas9-VP192 (gray) and multiplexed gRNAs compared to untreated HFF controls. FIG. 5c. Relative expression of mesenchymal-associated genes THY1 (left) and ZEB1 (right) in representative iPSC colonies (C1 or C2) approximately 40 days after nucleofection of either dCas9-NMS (blue) or dCas9-VP192 (gray) and multiplexed gRNAs compared to untreated HFF controls. FIG. 5d and FIG. 5e. Immunofluorescence microscopy of HFFs approximately 40 days after nucleofection of either dCas9-NMS or dCas9-VP192 and multiplexed gRNAs compared to untreated HFF controls (white scale bars, 100 μm). Cells were stained for the expression of pluripotency-associated cell surface markers SSEA4 (FIG. 5d, green) or TRA-1-81 (FIG. 5e, green). All cells were counterstained with DAPI for nuclear visualization.

FIGS. 6a-h. CRISPR-DREAM components are well tolerated in primary cells and compatible with viral delivery methods. FIGS. 6a-b. Immunofluorescence microscopy showing mCherry/EGFP expression levels in MSCs (FIG. 6a) and human T cells (FIG. 6b) 72 hrs after co-transduction of dCas9 in combination with either MCP-mCherry (control), MCP-eN3×9-T2A-EGFP, MCP-MSN-T2A-EGFP, MCP-NMS-T2A-EGFP, or MCP-VPR-T2A-EGFP respectively (white scale bars, 250 μm for MSCs, 100 μm for T cells). MCP-fusion vectors also contain a U6 driven gRNA expression cassette and either a TTN (MSCs) or CARD9 (T cells). FIGS. 6c-d. Relative expression of TTN (FIG. 6c) or CARD9 (FIG. 6d) in MSCs and T cells, respectively, 3 days after lentiviral co-transduction using indicated components. FIG. 6e. AAV constructs used for dual-delivery of CRISPR-DREAM components are schematically depicted. The EFGP control vector is shown (top) along with the hSyn promoter driven SpdCas9 vector (middle), which consists of a modified WPRE/polyA sequence (W3SL). The U6 promoter driven gRNA expressing vector (bottom) is also shown and also encodes MCP fused to MSN, which is driven by the hSyn promoter. FIG. 6f. Agrp gene activation in mouse primary cortical neurons using the dual AAV8 transduced CRISPR-DREAM system described (in FIG. 6e) 5 days post transduction. FIG. 6g. All-in-one (AIO) SadCas9-based AAV vectors are schematically depicted. AIO vectors consist of M11 promoter driven gRNA cassettes and either SCP1 (top) or EFS (bottom) promoter driven NMS-SadCas9. A modified WPRE/polyA sequence (CW3SA) was used in the AIO vectors. FIG. 6h. Agrp gene activation in mouse primary cortical neurons transduced with AIO AAV vectors (in FIG. 6h) 5 days post transduction. Data are the result of at least 2 biological replicates. Error bars; SEM. *; P<0.05.

FIGS. 7a-g. Transactivation potency of serum responsive MTF TADs when recruited to human promoters via dCas9. FIG. 7a. Schematics showing Hippo and SRF-MRTF family proteins; YAP, the hyperactive YAP mutant (YAP S397A), TAZ, SRF, MRTF-A, MRTF-B and MYOCD proteins. TADs for each respective MTF, along with amino acid (aa) coordinates, are shown in light blue. FIG. 7b and FIG. 7c. OCT4 and IL1RN mRNA levels after the indicated TADs were fused to the N- or C-terminus of dCas9 targeted to each respective promoter using 4 pooled gRNAs. FIG. 7d and FIG. 7e. OCT4 and IL1RN mRNA levels after the indicated TADs were fused to the MCP protein and recruited via 4 pooled MS2 modified gRNAs and dCas9. MCP fused to the bipartite p65-HSF was used as a positive control. FIG. 7f and FIG. 7g. OCT4 and IL1RN mRNA levels after the indicated TADs were fused to scFv and recruited via dCas9 harboring 10×GCN4 C-terminal fusion protein (the SunTag system) along with 4 pooled standard gRNAs targeting each respective promoter. All samples were processed for QPCR analysis 72 hrs post-transfection in HEK293T cells and are the result of at least 2 biological replicates. Error bars; SEM.

FIGS. 8a-f. Comparison and versatility of gene activation potential between TADs from MRTF-A and MRTF-B. FIGS. 8a-b. OCT4 and IL1RN mRNA levels after the indicated TADs from MRTA-A (left) or MRTF-B (right) were recruited using the specified dCas9-based recruitment architecture (direct fusion, SunTag-based, or MCP-based) and 4 corresponding pooled gRNAs. FIGS. 8c-d. mRNA levels for indicated loci after TADs from MRTF-A or MRTF-B were recruited via dCas9 and targeted to promoters using pools of MS2 modified gRNAs (FIG. 8c; HBG1 and TTN promoters) or a single gRNA (FIG. 8d; SBNO2 and TBX5 promoters). FIGS. 8e-f. GRASLND long noncoding RNA (left) or NET1 eRNA (right) levels after TADs from MRTF-A or MRTF-B were recruited via dCas9 and targeted to each respective locus using 4 and 2 pooled MS2 modified gRNAs, respectively. All samples were processed for QPCR analysis 72 hrs post-transfection in HEK293T cells and are the result of at least 2 biological replicates. Error bars; SEM.

FIGS. 9a-g. Transactivation potency of oxidative stress responsive MTF TADs when recruited to human promoters via dCas9. FIG. 9a. TADs from the NRF2 protein are depicted schematically. eNRF2 is a fusion between Neh4 and Neh5 TADs separated by an 11 amino acid (aa) extended glycine-serine linker. The length of NRF2 and aa coordinates of individual TADs (Neh4 and Neh5) are shown. FIG. 9b and FIG. 9c. OCT4 and IL1RN mRNA levels after the indicated TADs were fused to the N- or C-terminus of dCas9 and targeted to each respective promoter using 4 pooled standard gRNAs. FIG. 9d and FIG. 9e. OCT4 and IL1RN mRNA levels after the indicated TADs were fused to the MCP protein and recruited via 4 pooled MS2 stem-loop modified gRNAs and dCas9. MCP fused to the bipartite p65-HSF was used as a positive control. FIG. 9f and FIG. 9g. OCT4 and IL1RN mRNA levels after the indicated TADs were fused to scFv and recruited via dCas9 harboring 10×GCN4 C-terminal fusion protein (the SunTag system) along with 4 pooled standard gRNAs targeting each respective promoter. All samples were processed for QPCR analysis 72 hrs post-transfection in HEK293T cells and are the result of at least 2 biological replicates. Error bars; SEM.

FIGS. 10a-e. Comparison and versatility of gene activation potential of the eNRF2 TAD. FIG. 10a and FIG. 10b. OCT4 and IL1RN mRNA levels after the eNRF2 TAD was recruited using the specified dCas9-based recruitment architecture (direct fusion, SunTag-based, or MCP-based) and 4 corresponding pooled gRNAs. FIG. 10c and FIG. 10d. mRNA levels for indicated loci after the eNRF2 TAD was recruited via dCas9 and targeted to promoters using pools of MS2 modified gRNAs (FIG. 10c; HBG1 and TTN promoters) or a single gRNA (FIG. 10d; SBNO2 and TBX5 promoters). FIG. 10e. GRASLND long noncoding RNA (left) or NET1 eRNA (right) levels after the eNRF2 TAD was recruited via dCas9 and targeted to each indicated locus using 4 and 2 pooled MS2 modified gRNAs, respectively. All samples were processed for QPCR analysis 72 hrs post-transfection in HEK293T cells and are the result of at least 3 biological replicates. Error bars; SEM.

FIGS. 11a-g. Transactivation potency of cytokine responsive MTF TADs when recruited to human promoters via dCas9. FIG. 11a. Schematics showing STAT family proteins STAT1, STAT2, STAT3, STAT4, STAT5 and STAT6. TADs for each respective protein are shown in light blue along with amino acid (aa) coordinates. FIG. 11b and FIG. 11c. OCT4 and IL1RN mRNA levels after the indicated TADs were fused to the N- or C-terminus of dCas9 targeted to each respective promoter using 4 pooled standard gRNAs. FIG. 11d and FIG. 11e. OCT4 and IL1RN mRNA levels after the indicated TADs were fused to the MCP protein and recruited via 4 pooled MS2 modified gRNAs and dCas9. MCP fused to the bipartite p65-HSF was used as a positive control. FIG. 11f and FIG. 11g. OCT4 and IL1RN mRNA levels after the indicated TADs were fused to scFv and recruited via dCas9 harboring a 10×GCN4 C-terminal fusion protein (the SunTag system) along with 4 pooled standard gRNAs targeting each respective promoter. All samples were processed for QPCR analysis 72 hrs post-transfection in HEK293T cells and are the result of at least 2 biological replicates. Error bars; SEM.

FIGS. 12-c. Bipartite fusions between MRTF and STAT TADs enhance transactivation potential. FIG. 12a. Schematic diagram showing the fusion of STAT1 through STAT6 TADs to the C- or N-terminus of MRTF-A or MRTF-B TADs. FIG. 12b. OCT4 mRNA activation when targeted by indicated bipartite MRTF-STAT TAD fusions relative to dCas9+MCP-mCherry. The dotted line indicates basal OCT4 expression in dCas9+MCP-mCherry transfected HEK293T cells. FIG. 12c. OCT4 mRNA activation potential of indicated bipartite MRTF-STAT TAD fusions relative to dCas9+MCP-MRTF-A. The dotted line indicates OCT4 expression in dCas9+MCP-MRTF-A transfected HEK293T cells. All samples were processed for QPCR analysis 72 hrs post-transfection and are the result of at least 2 biological replicates. Error bars; SEM.

FIGS. 13a-c. Fusion of eNRF2 to the C- or N-terminus of MRTF-A-STAT1 (MS) fusions further enhances transactivation potency. FIG. 13a. Schematic diagram showing tripartite MSN or NMS fusion proteins (base pair, bp sizes below) in conjunction with the MS2 binding protein; MCP. The eNRF2 TAD was either fused to the C-terminus or N-terminus of the bipartite MRTF-A-STAT1 TAD. FIG. 13b. OCT4 mRNA levels after targeting with dCas9, 4 MS2-modified gRNAs, and the indicated MS2-recruited tripartite TADs. FIG. 13c. OCT4 mRNA levels after targeting with dCas9 or indicated direct dCas9 C-terminal TAD fusions, 4 MS2-modified gRNAs, and the MS2-recruited tripartite MCP-MSN TAD. OCT4 activation levels are presented relative to the activation achieved by dCas9+MCP-MSN. All samples were processed for QPCR analysis 72 hrs post-transfection and are the result of at least 8 biological replicates in HEK293T cells. Error bars; SEM.

FIGS. 14a-m. CRISPR-DREAM activation of coding genes in HEK293T cells using pooled gRNAs. Relative expression levels of 13 different endogenous human genes after Control, DREAM, or SAM systems were targeted to their respective promoters using pools of gRNAs in HEK293T cells. All samples were processed for QPCR analysis 72 hrs post-transfection and are the result of at least 3 biological replicates. Error bars; SEM. *; P<0.05, **; P<0.01, ***; P<0.001. ns; not significant.

FIGS. 15a-i. CRISPR-DREAM activation of coding genes in HEK293T cells using single gRNAs. Relative expression levels of 9 different endogenous human genes 72 hrs after Control, DREAM, or SAM systems were targeted to their respective promoters using a single gRNA in HEK293T cells. All samples were processed for QPCR analysis 72 hrs post-transfection, and are the result of at least 3 biological replicates. Error bars; SEM. *; P<0.05, ***; P<0.001. ns; not significant.

FIGS. 16a-d. Comparison of gene activation potency between DREAM and SAM systems spanning ˜1 kb regions upstream from human TSSs. FIGS. 16a-b. The genomic regions (hg38) encompassing the human TTN (FIG. 16a) and FOXA3 (FIG. 16b) genes on chromosomes 2 and 19, respectively, are shown. Genes and isoforms are shown in dark blue; gRNA target regions are indicated by black lines and light blue highlighting. H3K27ac (from GSE174866), H3K27me3 (from DRX013192), and DNase Hypersensitivity Sites (DHSs; from GSE32970) are shown in green, red, and blue, respectively. Transcription Start Sites (TSSs) for each gene indicated by black arrows. FIG. 16c and FIG. 16d. Comparison of transactivation potency between DREAM and SAM systems when targeted to indicated sites within the TTN or FOXA3 promoters, respectively. 10 gRNAs were designed to tile across ˜1 kb upstream of respective promoter regions. Non-transfected HEK293T cells were used as control. All samples were processed for QPCR analysis 72 hrs post-transfection and are the result of at least 4 biological replicates. Error bars; SEM. *; P<0.05, **; P<0.01, ***; P<0.001. ns; not significant.

FIGS. 17a-e. CRISPR-DREAM mediated activation of HBG1/HBG2 is specific, robust, and potent. FIG. 17a. The genomic region encompassing the human HBG1 and HBG2 genes, along with two nearby genes BGLT3 and HBBP1 on chromosome 11 (hg38) is shown. Genes are shown in dark blue; gRNA target regions are indicated by black lines. H3K27ac (from GSE174866), H3K27me3 (from DRX013192), and DNase Hypersensitivity Sites (DHSs; from GSE32970) are shown in green, red, and blue, respectively. FIG. 17b. An MA plot generated from DESeq2 analysis 72 hrs after HEK293T cells were transiently co-transfected with dCas9-VPR and four HBG1/HBG2 promoter targeting gRNAs. mRNAs corresponding to HBG1 and HBG2 isoforms (statistically significant differentially expressed with a fold change (FC)>2 or <−2 and a false discovery rate (FDR)<0.05) are shown in deep gray. Red dots indicate other statistically significant differentially expressed genes (FC>2 or <−2, and FDR <0.05). FIG. 17c. Heatmap showing the normalized gene expression in counts per million (CPM) for HBG1 and HBG2 in HEK293T cells transfected with DREAM, SAM, or dCas9-VPR and a pool of 4 HBG1/HBG2 promoter targeting gRNAs (MS2 modified gRNAs for DREAM and SAM systems and standard gRNAs for dCas9-VPR). FIG. 17d. QPCR analysis showing HBG1 expression after targeting with DREAM, SAM, or dCas9-VPR in HEK293T cells. dCas9+MCP-mCherry was used as a control. All samples were processed for QPCR analysis 72 hrs post-transfection in HEK293T cells and are the result of at least 3 biological replicates. Error bars; SEM. FIG. 17e. Venn diagram showing all statistically significant differentially regulated genes (FC>2 or <−2, and FDR<0.05) in HEK293T cells after the HBG1/HBG2 promoters were targeted as above using the DREAM, SAM, or dCas9-VPR systems. Genes in red font are specific to indicated experimental system, and bolded genes are components of human TADs used in respective DREAM or SAM systems.

FIGS. 18a-f. CRISPR-DREAM is robust across diverse human cancer cell lines. Transactivation potencies of DREAM and SAM systems are shown when targeted to indicated human promoters in a lung adenocarcinoma cell line (A549; FIG. 18a), a breast cancer cell line (SK-BR-3; FIG. 18b), a bone osteosarcoma epithelial cell line (U2OS FIG. 18c), a colorectal carcinoma cell line (HCT-116; FIG. 18d), a myelogenous leukemia cell line (K562; FIG. 18e), and a cervical cancer cell line (HeLa; FIG. 18f). All samples were processed for QPCR analysis 72 hrs post-transfection and are the result of at least 3 biological replicates. Error bars; SEM. *; P<0.05, **; P<0.01, ***; P<0.001. ns; not significant.

FIGS. 19a-d. CRISPR-DREAM is robust across diverse karyotypically normal human cells. FIGS. 19a-c. Transactivation potencies of DREAM and SAM systems are shown when targeted to indicated human promoters in hTERT-MSCs (FIG. 19a), PBMCs (FIG. 19b), Human Foreskin Fibroblasts (HFF; FIG. 19c), or retinal pigmented epithelial cells (ARPE-19; FIG. 19d). All samples were processed for QPCR analysis 72 hrs post-transfection and are the result of at least 3 biological replicates. Error bars; SEM. *; P<0.05, **; P<0.01. ns; not significant.

FIGS. 20a-b. CRISPR-DREAM is robust in rodent cells. Transactivation potencies of DREAM and SAM systems are shown when targeted to indicated human promoters in murine NIH3T3 cells (FIG. 20a) or Chinese hamster ovary (CHO-K1) cells (FIG. 20b). All samples were processed for QPCR analysis 72 hrs post-transfection and are the result of at least 3 biological replicates. Error bars; SEM. *; P<0.05. ns; not significant.

FIGS. 21a-e. CRISPR-DREAM activates gene expression when targeted to enhancers, activates eRNAs, and activates lncRNAs in human cells. FIG. 21a. MYOD mRNA levels after DREAM or SAM systems were targeted to the MYOD distal regulatory region (DRR) using 4 MS2-modified gRNAs. b and c. eRNA levels induced after DREAM or SAM systems were targeted to the FKBP5 (FIG. 21b) or GREB1 (FIG. 21c) enhancer in HEK293T cells. FIGS. 21d-e. lncRNA levels induced after DREAM or SAM systems were targeted to the HOTAIR (FIG. 21d) or MALAT1 (FIG. 21e) lncRNA promoters in HEK293T cells. All samples were processed for QPCR analysis 72 hrs post-transfection and are the result of at least 4 biological replicates. Error bars; SEM. *; P<0.05, **; P<0.01. ns; not significant.

FIGS. 22a-e. Orthogonal CRISPR-DREAM systems are potent in Hela cells. FIGS. 22a-b. SadCas9-DREAM mediated transactivation when targeted to the promoters of 2 different endogenous genes (HBG1; FIG. 22a, and TTN; FIG. 22b) in comparison to SadCas9-SAM or SadCas9-VPR systems. FIG. 22c. RNA sequence of the MS2 loop containing gRNA designed for CjdCas9 and schematics of CjCas9 gRNA with MS2 loop (magnified in inset) incorporated within the tetraloop region. FIGS. 22d-e. CjdCas9-DREAM mediated transactivation when targeted to the promoters of 2 different endogenous genes (HBG1; FIG. 22d, and TTN; FIG. 22e) in comparison to CjdCas9-SAM or MiniCAFE systems. All samples were process for QPCR analysis 72 hrs post-transfection and are the result of at least 4 biological replicates. Error bars; SEM. *; P<0.05, **; P<0.01, ***; P<0.001. ns; not significant.

FIGS. 23a-j. Prediction, construction, and validation of transactivation potential among different 9aa TADs. FIGS. 23a-c. different 9aa TADs from MRTF-A (FIG. 23a), MRTF-B (FIG. 23b) or MYOCD (FIG. 23c) were predicted using the Nine Amino Acids Transactivation Domain 9aaTAD Prediction Tool (world-wide-web at med.muni.cz/9aaTAD/). The Inventors selected 9aa TADs that showed 100% matches to database predictions. FIG. 23d. OCT4 gene activation when indicated 9aa TADs were fused to MCP and then recruited to OCT4 promoter using dCas9 and a pool of 4 MS2-modified gRNAs. FIG. 23e. OCT4 gene activation when indicated bipartite TADs, built using heterotypic 1× 9aa TADs, were fused to MCP and recruited to the OCT4 promoter using dCas9 and a pool of 4 MS2-modified gRNAs. The blue bar (MCP-MYOCD.1-MYOCD.3) showed the highest gene activation among all 2× 9aa TADs and was selected for generating heterotypic 3× 9aa TADs. FIG. 23f. OCT4 gene activation when indicated tripartite TADs, built using heterotypic 2× 9aa TADs, were fused to MCP and recruited to the OCT4 promoter using dCas9 and a pool of 4 MS2-modified gRNAs. The purple bar (MCP-MRTF-B.3-MYOCD.1-MYOCD.3) showed the highest gene activation among all 3× 9aa TADs and was selected for further analysis. FIGS. 23g-h. Relative gene activation when the selected 3× 9aa TAD (MCP-MRTF-B.3-MYOCD.1-MYOCD.3) was fused to MCP and recruited to either the CD34 (FIG. 23g) or SBNO2 (FIG. 23h) promoter using dCas9 and a pool of 4 MS2-modified gRNAs or a single MS2-modified gRNA, respectively. FIGS. 23i-j. Levels of GRASLND lncRNA (FIG. 23i) or OCT4 mRNA (FIG. 23j) after the selected 3× 9aa was recruited via dCas9 and targeted to either the GRASLND promoter or OCT distal enhancer (OCT4-DE), respectively, using pools of 4 MS2-modified gRNAs. All samples were processed for QPCR analysis 72 hrs post-transfection in HEK293T cells and are the result of at least 2 biological replicates. Error bars; SEM.

FIGS. 24a-b. Construction and validation of the eN3×9 TAD domain for the mini-DREAM system. The 3× 9aa TADs derived from MRTF-B or MYOCD are depicted schematically. eNRF2 and different combinations of fusion architectures between indicated 3× 9aa TADs with eNRF2 fusions are also shown. 3× 9aa TADs were either cloned to the C- or N-terminal of eNRF2 and separated by either single glycine-serine linker (GS) or a 11 amino acid extended glycine-serine linker (Linker; see FIG. 9a). Respective aa sizes of each fusion proteins are also shown. b. transcriptional activation of OCT4 after 3× 9aa TAD, eNRF2, or indicated TAD fusions were recruited to the OCT4 promoter via dCas9 and 4 pooled of MS2 modified gRNAs. The CRISPR-DREAM system is shown for comparison. All samples were processed for QPCR analysis 72 hrs post-transfection in HEK293T cells and are the result of at least 4 biological replicates. Error bars; SEM.

FIGS. 25a-h. Design, construction, and validation of HNH domain-deleted dCas9 variants for the mini-DREAM system. FIG. 25a. Different HNH domain-deleted SpdCas9 variants, along with wildtype dCas9, are schematically depicted. Two different HNH domain-deleted SpCas9 variants (amino acid, aa 792-897, or aa 768-919 deleted, respectively) were selected for analysis and reconstructed using either no linker, a single glycine-serine linker, or an XTEN16 linker separating dCas9 protein segments. FIG. 25b. All HHN domain-deleted dCas9 variants were expressed in HEK293T cells and Western blotting was performed 72 hrs post-transfection in HEK293T cells using either anti-FLAG or anti-Cas9 antibodies (Tubulin was used as a loading control). FIG. 25c. OCT4 transactivation when MCP-MSN was recruited to the OCT4 promoter using different indicated HNH domain-deleted dCas9 variants and a pool of 4 MS2-modified gRNAs. FIGS. 25d-h. Comparison of transactivation potential between CRISPR-DREAM and selected HNH domain-deleted dCas9 (with no linker)+MCP-MSN at indicated endogenous loci. All samples were processed for QPCR analysis 72 hrs post-transfection in HEK293T cells and are the result of at least 4 biological replicates. Error bars; SEM.

FIGS. 26a-h. mini-DREAM and mini-DREAM Compact systems display robust transactivation potencies in HEK293T cells. FIGS. 26a-d. Transactivation potencies of mini-DREAM and CRISPR-DREAM systems are shown when targeted to the IL1RN promoter (FIG. 26a) using pooled MS2-modified gRNAs, the SBNO2 (FIG. 26b) or UPK3B promoters (FIG. 26c) using a single MS2-modified gRNA, respectively, and the OCT4 distal enhancer (DE; FIG. 26d) using pooled MS2-modified gRNAs. FIGS. 26e-g. Transactivation potencies of mini-DREAM and mini-DREAM Compact systems are shown when targeted to the IL1RN (FIG. 26e), OCT4 (FIG. 26f) or CD34 promoters (FIG. 26g), respectively, using pooled MS2-modified gRNAs. FIG. 26h. Transactivation potencies of mini-DREAM and mini-DREAM Compact systems are shown when targeted to the SBNO2 promoter using a single MS2-modified gRNA. All samples were processed for QPCR analysis 72 hrs post-transfection in HEK293T cells and are the result of at least 3 biological replicates. Error bars; SEM.

FIGS. 27a-e. Generating and validating tripartite TADs in direct fusion architectures. FIG. 27a. OCT4 mRNA levels after different dCas9 direct fusions were targeted to the OCT4 promoter using pooled gRNAs. Indicated direct fusions were generated by linking MSN or NMS domains to either the C-terminus, N-terminus, or both termini of dCas9, along with selected combinations also containing VP64 as indicated. FIG. 27b. The expression levels of dCas9, NMS-dCas9-VP64, dCas9-VPR are shown as detected by Western blotting in HEK293T cells 72 hrs post-transfection. FIG. 27c. Lengths (in bp) of different fusion proteins and modules are shown. FIG. 27d. Relative expression levels of 11 different endogenous human genes after dCas9, NMS-dCas9-VP64, or dCas9-VPR systems were targeted to their respective promoters/enhancers using pooled gRNAs. FIG. 27e. Relative expression levels of 7 different endogenous human genes after dCas9, NMS-dCas9-VP64, or dCas9-VPR systems were targeted to their respective promoters/enhancers using single gRNAs. All samples were processed for QPCR analysis 72-84 hrs post-transfection in HEK293T cells and are the result of at least 4 biological replicates. Error bars; SEM.

FIGS. 28a-c. The NMS effector domain is compatible with, and robust in, the dCas9 SunTag system. FIGS. 28a-c. OCT4 (FIG. 28a), TTN (FIG. 28b) and HBG1 (FIG. 28c) mRNA levels after the indicated TADs were fused to scFv and recruited via dCas9 harboring a 10×GCN4 C-terminal fusion protein (the SunTag system) along with 4 pooled gRNAs targeting each respective promoter. All samples were processed for QPCR analysis 72 hrs post-transfection in HEK293T cells and are the result of at least 3 biological replicates. Error bars; SEM. *; P<0.05, **; P<0.01. ns; not significant.

FIG. 29. The tripartite MSN and NMS TADs are portable to synthetic TALE DNA binding systems. Relative IL1RN mRNA levels after individual (A-D) or pooled (“all”) IL1RN promoter-targeting TALEs (TALE-VP64, TALE-p300, TALE-MSN, TALE-NMS and TALE-VPR) were co-transfected into HEK293T cells. All samples were processed for QPCR analysis 72 hrs post-transfection in HEK293T cells and are the result of at least 4 biological replicates. Error bars; SEM.

FIGS. 30a-i. The tripartite MSN and NMS TADs are portable to different Type I CRISPRa systems. a-d. IL1RN (FIG. 30a), OCT4 (FIG. 30b), TTN (FIG. 30c) or SBNO2 (FIG. 30d) mRNA activation when the indicated Cas6 fusion protein encoding plasmids (and plasmids encoding the other components of Eco-cascade complex) were targeted to each corresponding promoter using a single crRNA. FIG. 30e. NET1 eRNA activation when the indicated Cas6 fusion protein encoding plasmids (and plasmids encoding the other components of Eco-cascade complex) were targeted to respective enhancer using a single crRNA. FIG. 30f. Multiplexed activation of 3 protein coding genes (TTN, HBG1 and OCT4) following co-transfection of the indicated Cas6 fusion protein encoding plasmids (and plasmids encoding the other components of Eco-cascade complex) and a multiplexed crRNA expression plasmid encoding 1 crRNA/locus. FIG. 30g. The Type I CRISPR system derived from P. aeruginosa (Pae-Cascade) is schematically depicted along with a representative effector fused to the Csy3 protein subunit. FIGS. 30h-i. HBG1 (FIG. 30h) and IL1B (FIG. 30i) mRNA activation using the indicated Csy3 fusion proteins when targeted to each corresponding promoter using a single crRNA, respectively. All samples were processed for QPCR analysis 72 hrs post-transfection in HEK293T cells and are the result of at least 4 biological replicates. Error bars; SEM. *; P<0.05, **; P<0.01, ***; P<0.001. ns; not significant.

FIGS. 31a-g. The tripartite MSN and NMS TADs are portable to the dCas12a-based CRISPRa system. FIGS. 31a-c. ASCL1 (FIG. 31a), ZFP42/REX1 (FIG. 31b) and ILIR2 (FIG. 31c) transactivation after the indicated dCas12a fusion proteins were targeted to each corresponding promoter using 2 crRNAs per respective locus. FIGS. 31d-e. NPY1R (FIG. 31d) and HBB (FIG. 31e) mRNA activation after the indicated dCas 12a fusion proteins were targeted to each corresponding promoter using a single crRNA. FIG. 31f. Multiplexed activation of 4 indicated endogenous genes 72 hrs after co-transfection of indicated dCas12a fusion protein encoding plasmids and a single plasmid encoding an 8-crRNA expression array (2 crRNAs/gene promoter). FIG. 31g. The crRNA expression array encoding 20 crRNA targeting 16 loci used in main text FIG. 4i, is schematically depicted. All samples were processed for QPCR analysis 72 hrs post-transfection in HEK293T cells and are the result of at least 3 biological replicates. Error bars; SEM. *; P<0.05. ns; not significant.

FIGS. 32a-j. dCas9-NMS permits efficient in vitro reprogramming of human fibroblasts. FIG. 32a. Bar graph showing iPSC colonies (the total number of colonies per million HFFs) generated from HFFs nucleofected with either dCas9-NMS and dCas9-VP192 and a gRNA cocktail (see main text and methods). FIGS. 32b-f. Relative expression of pluripotency-associated genes NANOG (FIG. 32b), LIN28A (FIG. 32c), REX1 (FIG. 32d), CDH1 (FIG. 32e), FGF4 (FIG. 32f) in representative colonies (C1 or C2) ˜40 days after nucleofection of either dCas9-NMS (blue) or dCas9-VP192 (gray) and multiplexed gRNAs compared to untreated HFF controls. FIGS. 32g-i. Relative expression of mesenchymal-associated genes ZEB2 (FIG. 32g), TWIST1 (FIG. 32h) and SNAIL2 (FIG. 32i), in representative colonies (C1 or C2) ˜40 days after nucleofection of either dCas9-NMS (blue) or dCas9-VP192 (gray) and multiplexed gRNAs compared to untreated HFF controls. FIG. 32j. Immunofluorescence microscopy of HFFs ˜40 days after nucleofection of either dCas9-NMS or dCas9-VP192 and multiplexed gRNAs compared to untreated HFF controls (white scale bars, 100 μm). Cells were immuno-stained for the expression of pluripotency-associated cell surface marker TRA-1-60 (FIG. 32j, green). All cells were counterstained with DAPI to visualize the nucleus.

FIGS. 33a-b. Lentiviral titers are influenced by the presence of TAD domains. FIG. 33a. Schematic representation of different lentiviral vectors with their respective sizes shown (in bp). FIG. 33b. Titers of different lentiviruses used in this study. Each colored dot indicates a lentiviral titer from an independent preparation.

FIGS. 34a-b. Effect of lentiviral transduction upon primary T cell health. FIG. 34a. Flow cytometry plot showing 7AAD⁺/Annexin V⁺ cells (after enriching for EGFP positive cells) co-transduced with indicated lentiviral vectors. FIG. 34b. Bar graph showing the percentage of healthy primary T cells among the EGFP positive (from flow cytometry data) transduced with indicated lentiviral particles.

FIGS. 35a-f. Selection and validation of optimal Agrp gRNA and construct designs for dual and all-in-one (AIO) AAV vectors. FIG. 35a. The genomic region (mm10) encompassing the mouse Agrp gene on chromosomes 8 is shown. Agrp gene is shown in dark blue; SpdCas9 gRNA (1-15) target regions are indicated by black lines and light blue highlighting except for the most potent gRNA (gRNA 5), which was selected for future experiments and is shown in red. SadCas9 gRNA (1-3) target regions are indicated by black lines and light blue highlighting except most potent gRNA1, which was selected for future experiments was shown in purple. H3K27ac (from GSE10666), and DNase Hypersensitivity Sites (DHSs; from GSE37074) are shown in green, and blue, respectively. Transcription Start Sites (TSSs) for Agrp is indicated by black arrows. FIG. 35b. Comparison of transactivation potency of different gRNA targeting Agrp promoter using the DREAM system. 15 gRNAs were designed to tile across ˜1.5 kb upstream of the Agrp promoter. Non-transfected Neuro-2A cells were used as a control. FIG. 35c. Dual AAV plasmid (comprising CRISPR-DREAM and Agrp gRNA5) mediated Agrp induction in Neuro-2A cells. FIG. 35d. Immunofluorescence microscopy showing EGFP expression in AAV8-EGFP transduced (2.5×10⁴ vg/cell) mouse primary cortical neurons 72 hrs post-transduction (red scale bars, 100 μm). FIG. 35e. Agrp induction in Neuro-2A cells 72 hrs after transfection of the AIO AAV construct consisting of the SCP1 promoter driven NMS-SadCas9. FIG. 35f. Agrp induction in Neuro-2A cells 72 hrs after transfection of the AIO AAV construct consisting of EFS promoter driven NMS-SadCas9.

### DESCRIPTION OF ILLUSTRATIVE EMBODIMENTS

The engineered upregulation of gene expression depends on seamless design, rational architecture and precise targeting of engineered transcription factors or epigenetic modifiers to the gene regulatory element of interest. Despite much progress in developing multipartite transcription factors and recruitment strategies to form multimeric complex at the target DNA sequence in human cells, there remain few options in the synthetic transcription factor (TF) toolbox.

Here, the inventors quantify the endogenous transactivation potency of dozens of different TADs derived from human MTFs in different combinations and across various dCas-based recruitment architectures. The inventors use these data to design new multipartite transactivation modules, called MSN, NMS, and eN3×9 and the inventors further apply the MSN and NMS effectors to build the CRISPR-dCas9 recruited enhanced activation module (DREAM) platform. The inventors demonstrate that CRISPR-DREAM potently stimulates transcription in primary human cells and cancer cell lines, as well as in murine and CHO cells.

The inventors also show that CRISPR-DREAM activates different classes of RNAs spanning diverse regulatory elements within the human genome. Further, the inventors find that the MSN/NMS effectors are portable to smaller engineered dCas9 variants, natural orthologues of dCas9, dCas12a, Type I CRISPR/Cas systems, and TALE and ZF proteins. Moreover, the inventors demonstrate that a dCas12a-NMS fusion enables superior multiplexing transactivation capabilities compared to existing systems.

The inventors also show that dCas9-NMS efficiently reprograms human fibroblasts to induced pluripotency and the inventors leverage the compact size of these new effectors to build potent dual and all-in-one CRISPRa AAVs. Finally, the inventors demonstrate that MSN, NMS, and eN3×9 are better tolerated than viral-based TADs in primary human MSCs and T cells. Overall, the engineered transactivation modules that the inventors have developed here are small, highly potent, devoid of viral sequences, versatile across programmable DNA binding systems, and enable robust multiplexed transactivation in human cells-important features that can be leveraged to test new biological hypotheses and engineer complex cellular functions. These and other aspects of the disclosure are described in detail below.

### I. Transcriptional Activators

Mechanosensitive transcription factors (MTFs) are highly regulated robust and efficient transcriptional modulators, which respond to mechanical cues or external ligands. Upon activation they can be shuttled to the cell nucleus, rapidly induce transcription and then subsequently can be exported from nucleus. These dynamics are controlled by the nature and the intensity of stimulation. MTFs coordinate this rapid transcription by engaging many nuclear factors including RNA polymerase, histone writers, readers, and/or erasers. Here, the inventors evaluated and selected particular TADs from a variety of factors including serum regulated (YAP-TAZ-TEAD and SRF-MRTF/MYOCD) transcription factors, cytokine regulated JAK-STAT family transcription factors and oxidative stress/antioxidant regulated NRF2. A discussion of these factors and the design of new recombinant transcription regulators is provided below.

### A. Transcription Factors and Activation Domains

A transcription factor (TF) (or sequence-specific DNA-binding factor) is a protein that controls the rate of transcription of genetic information from DNA to messenger RNA, by binding to a specific DNA sequence. The function of TFs is to regulate gene expression to make sure that they are expressed in the right cell at the right time and in the right amount throughout the life of the cell and the organism. Groups of TFs function in a coordinated fashion to direct cell division, cell growth, and cell death throughout life; cell migration and organization during embryonic development; and intermittently in response to signals from outside the cell, such as a hormone. Transcription factors are members of the proteome as well as regulome.

TFs work alone or with other proteins in a complex, by promoting (as an activator), or blocking (as a repressor) the recruitment of RNA polymerase (the enzyme that performs the transcription of genetic information from DNA to RNA) to specific genes.

A defining feature of TFs is that they contain at least one DNA-binding domain (DBD), which attaches to a specific sequence of DNA adjacent to the genes that they regulate. TFs are grouped into classes based on their DBDs. Other proteins such as coactivators, chromatin remodelers, histone acetyltransferases, histone deacetylases, kinases, and methylases are also essential to gene regulation, but lack DNA-binding domains, and therefore are not TFs.

Transcription factors are essential for the regulation of gene expression and are, as a consequence, found in all living organisms. The number of transcription factors found within an organism increases with genome size, and larger genomes tend to have more transcription factors per gene.

There are approximately 2800 proteins in the human genome that contain DNA-binding domains, and 1600 of these are presumed to function as transcription factors, though other studies indicate it to be a smaller number. Therefore, approximately 10% of genes in the genome code for transcription factors, which makes this family the single largest family of human proteins. Furthermore, genes are often flanked by several binding sites for distinct transcription factors, and efficient expression of each of these genes requires the cooperative action of several different transcription factors (see, for example, hepatocyte nuclear factors). Hence, the combinatorial use of a subset of the approximately 2000 human transcription factors easily account for the unique regulation of each gene in the human genome during development.

Transcription factors bind to either enhancer or promoter regions of DNA adjacent to the genes that they regulate. Depending on the transcription factor, the transcription of the adjacent gene is either up- or down-regulated. Transcription factors use a variety of mechanisms for the regulation of gene expression. These mechanisms include:
stabilizing or blocking the binding of RNA polymerase to DNA
catalyzing the acetylation or deacetylation of histone proteins
histone acetyltransferase (HAT) activity
histone deacetylase (HDAC) activity
recruiting coactivator or corepressor proteins to the transcription factor DNA complex

Transactivation domains or trans-activating domains (TADs) are transcription factor scaffold domains which contain binding sites for other proteins such as transcription coregulators. These binding sites are frequently referred to as activation functions (AFs). TADs are named after their amino acid composition. These amino acids are either essential for the activity or simply the most abundant in the TAD. Transactivation by the Gal4 transcription factor is mediated by acidic amino acids, whereas hydrophobic residues in Gcn4 play a similar role. Hence, the TADs in Gal4 and Gcn4 are referred to as acidic or hydrophobic, respectively.
In general, there are four classes of TADs:
acidic domains (called also “acid blobs” or “negative noodles”, rich in D and E amino acids, present in Gal4, Gcn4 and VP16).
glutamine-rich domains (contains multiple repetitions like “QQQXXXQQQ” (SEQ ID NO: 1), present in SP1)
proline-rich domains (contains repetitions like “PPPXXXPPP” (SEQ ID NO: 2) present in c-jun, AP2 and Oct-2)
isoleucine-rich domains (repetitions “IIXXII” (SEQ ID NO: 3), present in NTF-1)
   Alternatively, since similar amino acid compositions does not necessarily mean similar activation pathways, TADs can be grouped by the process they stimulate, either initiation or elongation.

### B. MRTF-A

MRTF-A (myocardin related transcription factor A), also known as MKL/megakaryoblastic leukemia 1 is a protein that in humans is encoded by the MKL1 gene. The protein encoded by this gene is regulated by the actin cytoskeleton and is shuttled between the cytoplasm and the nucleus in response to actin dynamics. In the nucleus, it coactivates the transcription factor serum response factor, a key regulator of smooth muscle cell differentiation, in an interaction mediated by its Basic domain. It is closely related to MKL2 and myocardin, with which it shares five key conserved structural domains. This gene is involved in a specific translocation event that creates a fusion of this gene and the RNA-binding motif protein-15 gene. This translocation has been associated with acute megakaryocytic leukemia. It also functions in the process of normal megakaryocyte maturation.

Reference sequences for MRTF-A mRNA and protein can be found at NM_001282660 and NP_001269589, respectively.

### C. STAT1

Signal transducer and activator of transcription 1 (STAT1) is a transcription factor which in humans is encoded by the STAT1 gene. It is a member of the STAT protein family. All STAT molecules are phosphorylated by receptor associated kinases, that causes activation, dimerization by forming homo- or heterodimers and finally translocate to nucleus to work as transcription factors. Specifically, STAT1 can be activated by several ligands such as Interferon alpha (IFNα), Interferon gamma (IFNγ), Epidermal Growth Factor (EGF), Platelet Derived Growth Factor (PDGF), Interleukin 6 (IL-6), or IL-27.

Type I interferons (IFN-α, IFN-β) bind to receptors, cause signaling via kinases, phosphorylate and activate the Jak kinases TYK2 and JAK1 and STAT1 and STAT2. STAT molecules form dimers and bind to ISGF3G/IRF-9, which is Interferon stimulated gene factor 3 complex with Interferon regulatory Factor 9. This allows STAT1 to enter the nucleus. STAT1 has a key role in many gene expressions that cause survival of the cell, viability or pathogen response. There are two possible transcripts (due to alternative splicing) that encode 2 isoforms of STAT1. STAT1α, the full-length version of the protein, is the main active isoform, responsible for most of the known functions of STAT1. STAT1β, which lacks a portion of the C-terminus of the protein, is less-studied, but has variously been reported to negatively regulate activation of STAT1 or to mediate IFN-γ-dependent anti-tumor and anti-infection activities.

STAT1 is involved in upregulating genes due to a signal by either type I, type II, or type III interferons. In response to IFN-γ stimulation, STAT1 forms homodimers or heterodimers with STAT3 that bind to the GAS (Interferon-Gamma-Activated Sequence) promoter element; in response to either IFN-α or IFN-β stimulation, STAT1 forms a heterodimer with STAT2 that can bind the ISRE (Interferon-Stimulated Response Element) promoter element. In either case, binding of the promoter element leads to an increased expression of ISG (Interferon-Stimulated Genes).

Reference sequences for STAT1 mRNA and protein can be found at NM_007315 and NP_009330, respectively.

### D. NRF2

Nuclear factor erythroid 2-related factor 2 (NRF2), also known as nuclear factor erythroid-derived 2-like 2, is a transcription factor that in humans is encoded by the NFE2L2 gene. NRF2 is a basic leucine zipper (bZIP) protein that may regulate the expression of antioxidant proteins that protect against oxidative damage triggered by injury and inflammation, according to preliminary research. In vitro, NRF2 binds to antioxidant response elements (AREs) in the nucleus leading to transcription of ARE genes. NRF2 increases heme oxygenase 1 leading to an increase in phase II enzymes in vitro. NRF2 also inhibits the NLRP3 inflammasome.

NRF2 appears to participate in a complex regulatory network and performs a pleiotropic role in the regulation of metabolism, inflammation, autophagy, proteostasis, mitochondrial physiology, and immune responses. Several drugs that stimulate the NFE2L2 pathway are being studied for treatment of diseases that are caused by oxidative stress. A mechanism for hormetic dose responses is proposed in which Nrf2 may serve as an hormetic mediator that mediates a vast spectrum of chemopreventive processes.

NRF2 is a basic leucine zipper (bZip) transcription factor with a Cap “n” Collar (CNC) structure. NRF2 possesses six highly conserved domains called NRF2-ECH homology (Neh) domains. The Neh1 domain is a CNC-bZIP domain that allows Nrf2 to heterodimerize with small Maf proteins (MAFF, MAFG, MAFK). The Neh2 domain allows for binding of NRF2 to its cytosolic repressor Keap1. The Neh3 domain may play a role in NRF2 protein stability and may act as a transactivation domain, interacting with component of the transcriptional apparatus. The Neh4 and Neh5 domains also act as transactivation domains but bind to a different protein called cAMP Response Element Binding Protein (CREB), which possesses intrinsic histone acetyltransferase activity. The Neh6 domain may contain a degron that is involved in a redox-insensitive process of degradation of NRF2. This occurs even in stressed cells, which normally extend the half-life of NRF2 protein relative to unstressed conditions by suppressing other degradation pathways.

Reference sequences for NRF2 mRNA and protein can be found at NM_006164 and NP_001138884, respectively.

### E. MSN and NMS

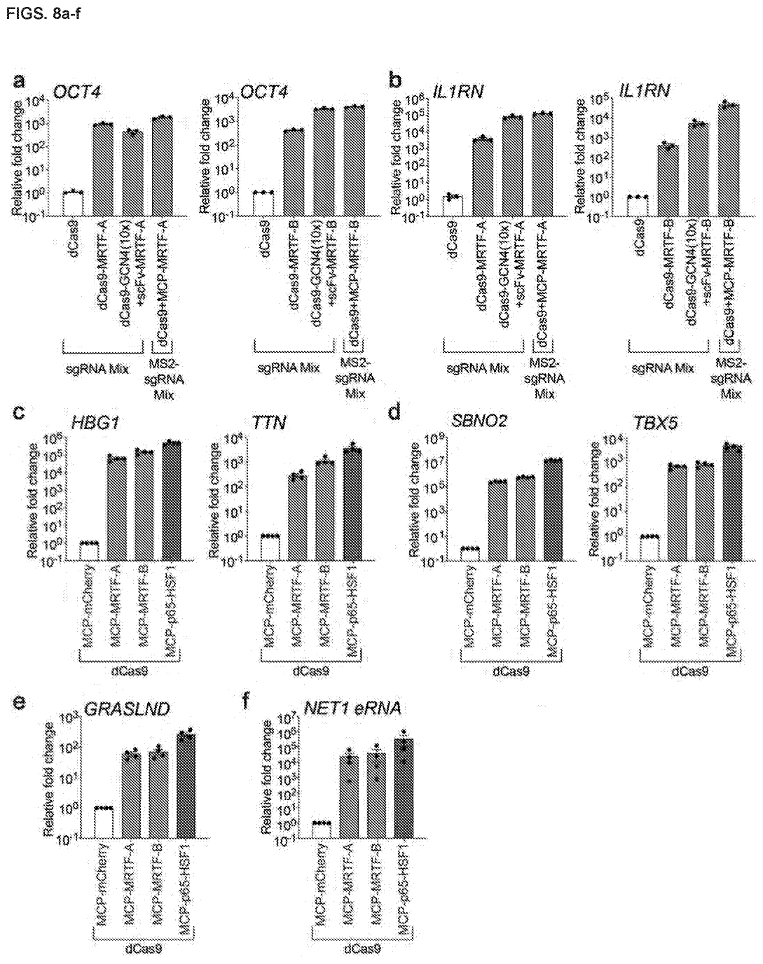

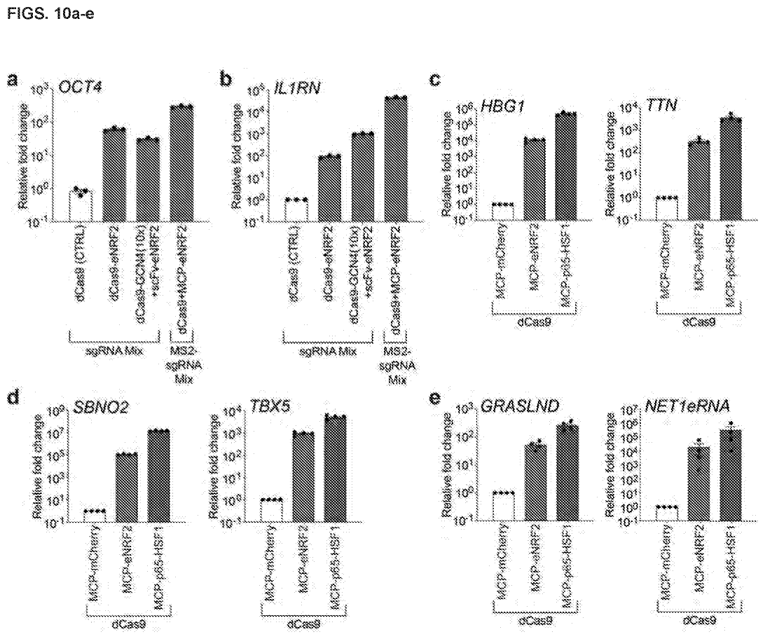

As discussed above, the inventors explored the transcription activation properties of a variety TAD domains from human transcription factors. After selecting the most potent, they examined all possible anchoring positions (direct fusion in N-terminal and C-terminal, MS2-MCP and SunTag) with a dCas9-sgRNA complex. They broadly divided these transcription factors into general categories based on their regulation of transcription and tested their ability to activate transcription in all anchoring architectures. Surprisingly, none of the TADs of STAT family members alone were able to activate transcription from the inventors' testbed. Among the three TAD domains of NRF2, two fused TAD domains of NRF2 namely Neh4 and Neh5 (designated eNRF2) were the most promising. In addition, MS2-MCP mediated recruitment showed significantly higher degree of upregulation among all anchoring architectures both for MRTF-A, MRTF-B and eNRF2. To assess the broad effectiveness of these TADs (MRTF-A, MRTF-B and eNRF2), the inventors tested their efficacy on endogenous protein coding genes in pooled gRNA settings (HBG1), single gRNA settings (SBNO2), LncRNA (GRASLND) and eRNA (NET1) and as expected these selected TADs can upregulate all tested target genes from 5 to 2000-fold (FIGS. 8a-f and FIGS. 10a-e).

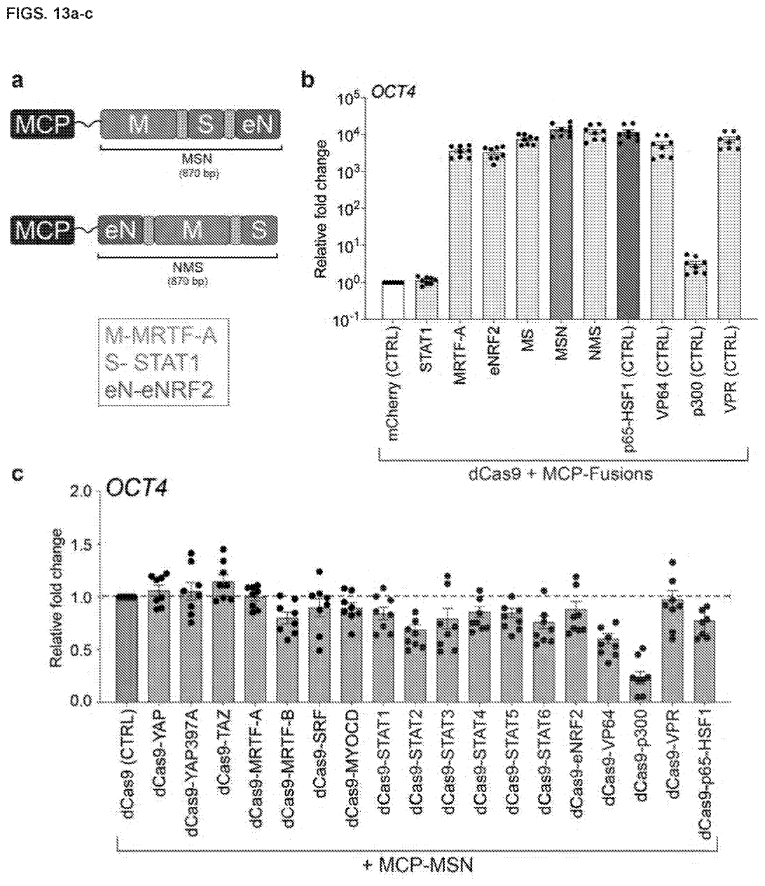

To further increase transcriptional activity, the inventors made tripartite fusions by fusing eNRF2, MRTF-A and STAT1 and constructed two highly potent engineered transcription factors designated MSN (MRTF-A-STAT1-eNRF2) and NMS (eNRF2-MRTF-A-STAT1). These molecules were tested and compared for gene activation potential with all state of art CRISPR based activators (MCP-p65-HSF1, MCP-VP64, MCP-VPR, MCP-p300), MCP-MRTF-a-STAT1-eNRF2 (MCP-MSN) showed higher activation than all as tested on OCT4 locus (FIGS. 13a-c).

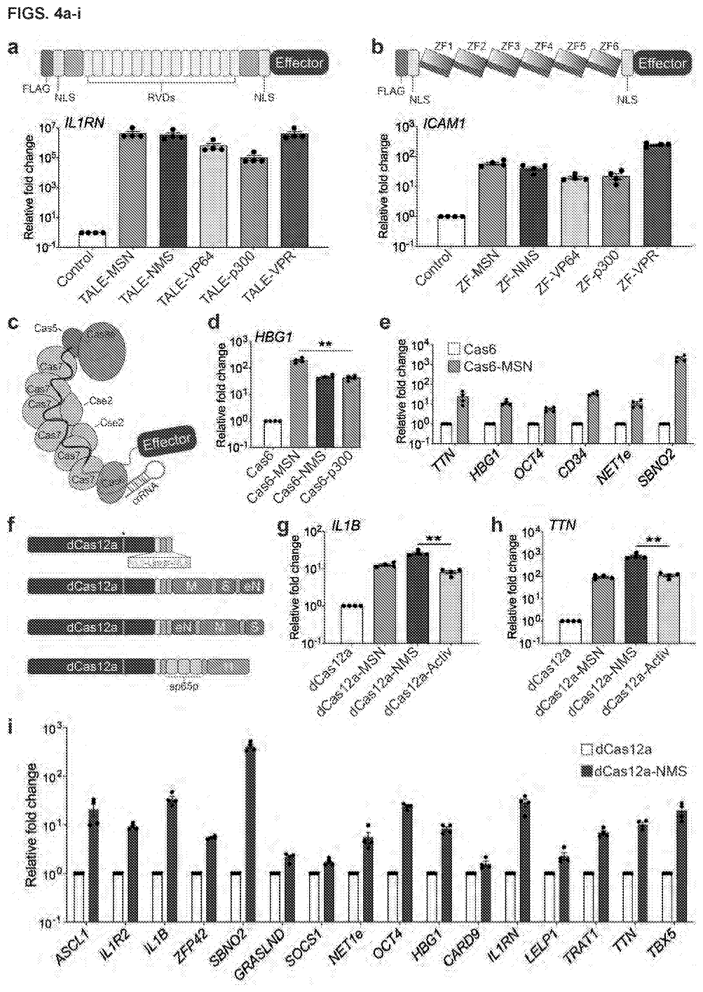

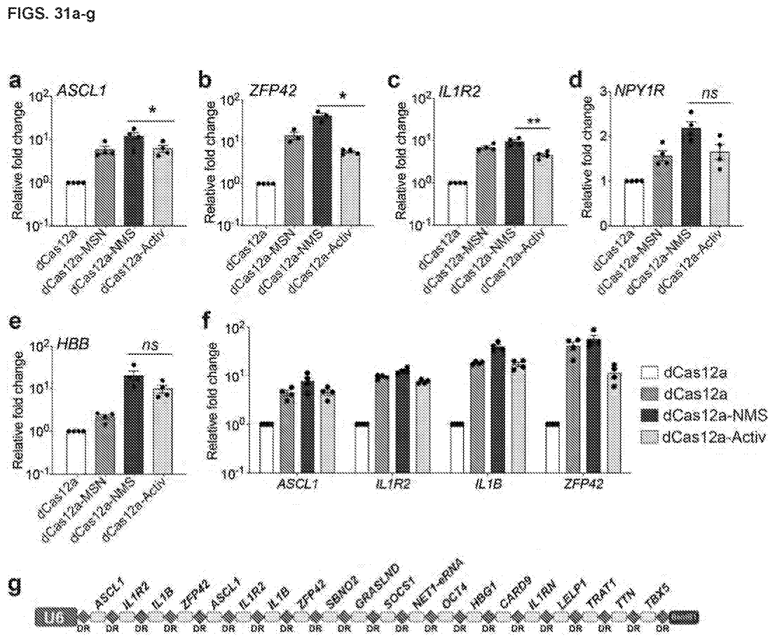

The inventors then investigated the potency of tripartite fusions (both MSN and NMS) by transferring them to another robust, versatile, easily programmable and multiplexable orthogonal system, namely, dCas12a. These were compared against available gold standard activator dCas12a-[Activ] and the result clearly demonstrate that dCas12a-NMS is able to induce transcription comparable or better than dCas12a-[Activ] and in tested ASCL1, ILIR2 loci (2 crRNA for each gene). Finally, it has been shown that Cas12a can process up to 20 crRNA and can activate 10 different genes, so the inventors took similar strategy and cloned 20 crRNA in an array targeting 16 different endogenous genes, targeting either promoter, enhancer, eRNA and LncRNA and the data showed the activation of 16 genes using dCas12a-NMS (FIGS. 4f-i and Supplementary FIGS. 31a-g).

Recently, the prototypic and well-studied Type I CRISPR system (E. coli K12) was engineered to robustly modulate transcription from endogenous loci. To leverage the efficacy of MSN and NMS domains and Type I CRISPR system, the inventors transferred MSN and NMS domains to Cas6 and compared its efficiency against already benchmarked Cas6-p300 system. These data demonstrate that Cas6-MSN acts superior to Cas6-p300 in the targeted TTN and HBG1 loci. Further, like dCas12a, Type I cascade system can process its own crRNA array and shown to activate 2 genes in arrayed crRNA settings, here, the inventors further extend the crRNA array up to 6, targeting 4 different genes and found that MSN is superior to p300 in multiplex activation platform (FIGS. 4c-e).

### II. Genomic Regulatory Element Targeting Domains, RNA Binding Domains and Linkers

As discussed above, the utility of the TADs described above has been demonstrated using a variety of targeting domains. While the precise nature and function of the targeting domains is secondary, and virtually any such domain could function, the following discussion highlights highly relevant examples. In addition, an optional element further includes RNA binding elements such as MCPs, PCPs and Pumilio proteins. These elements would expand the toolbox of recruitment strategies of these domains, enabling the targeting of multiple effectors in combination with the MSN and NMS.

### A. Cas Proteins

Cas (CRISPR associated protein) molecules play a vital role in the immunological defense of certain bacteria against DNA viruses and plasmids and is heavily utilized in genetic engineering applications. Its main function is to cut DNA and thereby alter a cell's genome.

Cas9 is a perhaps the most studied of all the Cas molecules. It is a dual RNA-guided DNA endonuclease enzyme associated with the Clustered Regularly Interspaced Short Palindromic Repeats (CRISPR) adaptive immune system in Streptococcus pyogenes. S. pyogenes utilizes CRISPR to memorize and Cas9 to later interrogate and cleave foreign DNA, such as invading bacteriophage DNA or plasmid DNA. Cas9 performs this interrogation by unwinding foreign DNA and checking for sites complementary to the 20 bP spacer region of the guide RNA. If the DNA substrate is complementary to the guide RNA, Cas9 cleaves the invading DNA. In this sense, the CRISPR-Cas9 mechanism has a number of parallels with the RNA interference (RNAi) mechanism in eukaryotes.

Apart from its original function in bacterial immunity, the Cas9 protein has been heavily utilized as a genome engineering tool to induce site-directed double-strand breaks in DNA. These breaks can lead to gene inactivation or the introduction of heterologous genes through non-homologous end joining and homologous recombination respectively in many laboratory model organisms. Alongside zinc finger nucleases and Transcription activator-like effector nuclease (TALEN) proteins, Cas9 is becoming a prominent tool in the field of genome editing.

Cas9 has gained traction in recent years because it can cleave nearly any sequence complementary to the guide RNA. Because the target specificity of Cas9 stems from the guide RNA: DNA complementarity and not modifications to the protein itself (like TALENs and zinc fingers), engineering Cas9 to target new DNA is straightforward. Versions of Cas9 that bind but do not cleave cognate DNA can be used to locate transcriptional activator or repressors to specific DNA sequences in order to control transcriptional activation and repression. Native Cas9 requires a guide RNA composed of two disparate RNAs that associate—the CRISPR RNA (crRNA), and the trans-activating crRNA (tracrRNA). Cas9 targeting has been simplified through the engineering of a chimeric single guide RNA (chiRNA).

Other useful Cas proteins include Cas6, AsdCas12a, SpdCas9, CjdCas9, and SadCas9.

### B. TALE DNA Binding Domain

TAL (transcription activator-like) effectors (often referred to as TALEs, but not to be confused with the three amino acid loop extension homeobox class of proteins) are proteins secreted by some β- and γ-proteobacteria. Most of these are Xanthomonads. Plant pathogenic Xanthomonas bacteria are especially known for TALEs, produced via their type III secretion system. These proteins can bind promoter sequences in the host plant and activate the expression of plant genes that aid bacterial infection. They recognize plant DNA sequences through a central repeat domain consisting of a variable number of ˜34 amino acid repeats. There appears to be a one-to-one correspondence between the identity of two critical amino acids in each repeat and each DNA base in the target sequence. These proteins are interesting to researchers both for their role in disease of important crop species and the relative ease of retargeting them to bind new DNA sequences. Similar proteins can be found in the pathogenic bacterium Ralstonia solanacearum and Burkholderia rhizoxinica, as well as yet unidentified marine microorganisms. The term TALE-likes is used to refer to the putative protein family encompassing the TALEs and these related proteins.

The most distinctive characteristic of TAL effectors is a central repeat domain containing between 1.5 and 33.5 repeats that are usually 34 residues in length (the C-terminal repeat is generally shorter and referred to as a “half repeat”). A typical repeat sequence is LTPEQVVAIASHDGGKQALETVQRLLPVLCQAHG (SEQ ID NO: 4), but the residues at the 12th and 13th positions are hypervariable (these two amino acids are also known as the repeat variable di-residue or RVD). There is a simple relationship between the identity of these two residues in sequential repeats and sequential DNA bases in the TAL effector's target site. The crystal structure of a TAL effector bound to DNA indicates that each repeat comprises two alpha helices and a short RVD-containing loop where the second residue of the RVD makes sequence-specific DNA contacts while the first residue of the RVD stabilizes the RVD-containing loop. Target sites of TAL effectors also tend to include a thymine flanking the 5′ base targeted by the first repeat; this appears to be due to a contact between this T and a conserved tryptophan in the region N-terminal of the central repeat domain. However, this “zero” position does not always contain a thymine, as some scaffolds are more permissive.

TAL effectors can induce susceptibility genes that are members of the NODULIN3 (N3) gene family. These genes are essential for the development of the disease. In rice two genes, Os-8N3 and Os-11N3, are induced by TAL effectors. Os-8N3 is induced by PthXo1 and Os-11N3 is induced by PthXo3 and AvrXa7. Two hypotheses exist about possible functions for N3 proteins-first, that they are involved in copper transport, resulting in detoxification of the environment for bacteria (the reduction in copper level facilitates bacterial growth), and second, that they are involved in glucose transport, facilitating glucose flow (this mechanism provides nutrients to bacteria and stimulates pathogen growth and virulence).

This simple correspondence between amino acids in TAL effectors and DNA bases in their target sites makes them useful for protein engineering applications. Numerous groups have designed artificial TAL effectors capable of recognizing new DNA sequences in a variety of experimental systems. Such engineered TAL effectors have been used to create artificial transcription factors that can be used to target and activate or repress endogenous genes in tomato, Arabidopsis thaliana, and human cells.

Genetic constructs to encode TAL effector-based proteins can be made using either conventional gene synthesis or modular assembly. A plasmid kit for assembling custom TALEN and other TAL effector constructs is available through the public, not-for-profit repository Addgene. Webpages providing access to public software, protocols, and other resources for TAL effector-DNA targeting applications include the TAL Effector-Nucleotide Targeter and taleffectors.com.

Engineered TAL effectors can also be fused to the cleavage domain of FokI to create TAL effector nucleases (TALEN) or to meganucleases (nucleases with longer recognition sites) to create “megaTALs.” Such fusions share some properties with zinc finger nucleases and may be useful for genetic engineering and gene therapy applications. TALEN-based approaches are used in the emerging fields of gene editing and genome engineering. TALE-induced non-homologous end joining modification has been used to produce novel disease resistance in rice.

### C. Zinc Finger DNA Binding Domains

A zinc finger is a small protein structural motif that is characterized by the coordination of one or more zinc ions (Zn²⁺) to stabilize the fold. It was originally coined to describe the finger-like appearance of a hypothesized structure from the African clawed frog (Xenopus laevis) transcription factor IIIA. However, it has been found to encompass a wide variety of differing protein structures in eukaryotic cells. Xenopus laevis TFIIIA was originally demonstrated to contain zinc and require the metal for function in 1983, the first such reported zinc requirement for a gene regulatory protein followed soon thereafter by the Krüppel factor in Drosophila. It often appears as a metal-binding domain in multi-domain proteins.

Proteins that contain zinc fingers (zinc finger proteins) are classified into several different structural families. Unlike many other clearly defined supersecondary structures such as Greek keys or β hairpins, there are a number of types of zinc fingers, each with a unique three-dimensional architecture. A particular zinc finger protein's class is determined by this three-dimensional structure, but it can also be recognized based on the primary structure of the protein or the identity of the ligands coordinating the zinc ion. In spite of the large variety of these proteins, however, the vast majority typically function as interaction modules that bind DNA, RNA, proteins, or other small, useful molecules, and variations in structure serve primarily to alter the binding specificity of a particular protein.

Since their original discovery and the elucidation of their structure, these interaction modules have proven ubiquitous in the biological world and may be found in 3% of the genes of the human genome. In addition, zinc fingers have become extremely useful in various therapeutic and research capacities. Engineering zinc fingers to have an affinity for a specific sequence is an area of active research, and zinc finger nucleases and zinc finger transcription factors are two of the most important applications of this to be realized to date.

Zinc finger (Znf) domains are relatively small protein motifs that contain multiple finger-like protrusions that make tandem contacts with their target molecule. Some of these domains bind zinc, but many do not, instead binding other metals such as iron, or no metal at all. For example, some family members form salt bridges to stabilise the finger-like folds. They were first identified as a DNA-binding motif in transcription factor TFIIIA from Xenopus laevis (African clawed frog), however they are now recognised to bind DNA, RNA, protein, and/or lipid substrates. Their binding properties depend on the amino acid sequence of the finger domains and on the linker between fingers, as well as on the higher-order structures and the number of fingers. Znf domains are often found in clusters, where fingers can have different binding specificities. Znf motifs occur in several unrelated protein superfamilies, varying in both sequence and structure. They display considerable versatility in binding modes, even between members of the same class (e.g., some bind DNA, others protein), suggesting that Znf motifs are stable scaffolds that have evolved specialised functions. For example, Znf-containing proteins function in gene transcription, translation, mRNA trafficking, cytoskeleton organization, epithelial development, cell adhesion, protein folding, chromatin remodeling, and zinc sensing, to name but a few. Zinc-binding motifs are stable structures, and they rarely undergo conformational changes upon binding their target.

Initially, the term zinc finger was used solely to describe DNA-binding motif found in Xenopus laevis; however, it is now used to refer to any number of structures related by their coordination of a zinc ion. In general, zinc fingers coordinate zinc ions with a combination of cysteine and histidine residues. Originally, the number and order of these residues was used to classify different types of zinc fingers (e.g., Cys2His2, Cys4, and Cys6). More recently, a more systematic method has been used to classify zinc finger proteins instead. This method classifies zinc finger proteins into “fold groups” based on the overall shape of the protein backbone in the folded domain. The most common “fold groups” of zinc fingers are the Cys2His2-like (the “classic zinc finger”), treble clef, and zinc ribbon.

Various protein engineering techniques can be used to alter the DNA-binding specificity of zinc fingers and tandem repeats of such engineered zinc fingers can be used to target desired genomic DNA sequences. Fusing a second protein domain such as a transcriptional activator or repressor to an array of engineered zinc fingers that bind near the promoter of a given gene can be used to alter the transcription of that gene. Fusions between engineered zinc finger arrays and protein domains that cleave or otherwise modify DNA can also be used to target those activities to desired genomic loci. The most common applications for engineered zinc finger arrays include zinc finger transcription factors and zinc finger nucleases, but other applications have also been described. Typical engineered zinc finger arrays have between 3 and 6 individual zinc finger motifs and bind target sites ranging from 9 basepairs to 18 basepairs in length. Arrays with 6 zinc finger motifs are particularly attractive because they bind a target site that is long enough to have a good chance of being unique in a mammalian genome.

### D. Linkers

Linkers are short peptide segments that permit the “fusion” of two often larger peptide or polypeptide regions such that the functionalities of the larger regions are not impaired or physically constrained by direct linkage at their termini. Linkers are often characterized by polar uncharged or charged residues, flexibility (although some applications benefit from rigid linkers) and secondary structures of particular nature.

Flexible GS linkers contain, not surprisingly, glycine and serine residues, including GGS, GSSGSS (SEQ ID NO: 5), and GSSSSSS (SEQ ID NO: 6). A particular example is (GGGGS) 3 (SEQ ID NO: 7). Another linker, called XTEN, is a short (16 aa) flexible peptide segment with no specific structure.

### III. Recombinant Vector Systems

Systems using MSN and NMS can not only be delivered as proteins per se (after appropriate recombinant production in bacterial or eurkaryotic hosts) but by expression from genetic construct as well. Plasmids or linear DNA encoding the NMS/MSN construct and the necessary gene regulatory elements can be delivered by virus, nanoparticles, or other methods. Similarly, RNA encoding these constructs and necessary regulatory elements or RNA modifications can be delivered via similar vehicles.

Expression requires that appropriate signals be provided in the vectors and include various regulatory elements in addition to the such as enhancers/promoters from both viral and mammalian sources that drive expression of the genes of interest in cells. Elements designed to optimize messenger RNA stability and translatability in host cells also are defined.

Use of the term “expression cassette” is meant to include any type of genetic construct containing a nucleic acid coding for a gene product in which part or all of the nucleic acid encoding sequence is capable of being transcribed and translated, i.e., is under the control of a promoter. The phrase “under transcriptional control” means that the promoter is in the correct location and orientation in relation to the nucleic acid to control RNA polymerase initiation and expression of the gene. An “expression vector” is meant to include expression cassettes comprised in a genetic construct that is capable of replication, and thus including one or more of origins of replication, transcription termination signals, poly-A regions, selectable markers, and multipurpose cloning sites.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, viral promotes such as the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized. Further, selection of a promoter that is regulated in response to specific physiologic signals can permit inducible expression of the gene product.

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

### IV. Target Genes and Cells of Interest

The target cells in which the presently disclosed molecules can be used are virtually limitless. Of particular interest are diseased cells that do not express or have low expression of a particular gene. Others are cells where induction of gene expression will differentiate the cell into a cell needed by a host, such as for wound healing or recovery from a traumatic insult such as a stroke or myocardial infarction. The presently disclosed molecules are also of particular use in generating iPSCs by inducing gene expression patterns capable of de-differentiating cells such as fibroblasts. Further, other cell types include cells of the immune system or those with immunomodulatory potential, eye, central nervous system (CNS)-related, and/or muscle cells.

### V. Treatment of Disease

The present disclosure has the potential to treat genetic disorders, in particular disorders of haploinsufficiency. Haploinsufficiency describes a model of dominant gene action in diploid organisms, in which a single copy of the wild-type allele at a locus in heterozygous combination with a variant allele is insufficient to produce the wild-type phenotype. Haploinsufficiency may arise from a de novo or inherited loss-of-function mutation in the variant allele, such that it produces little or no gene product (often a protein). Although the other, standard allele still produces the standard amount of product, the total product is insufficient to produce the standard phenotype. This heterozygous genotype may result in a non- or sub-standard, deleterious, and (or) disease phenotype. Haploinsufficiency is the standard explanation for dominant deleterious alleles.

In the alternative case of haplosufficiency, the loss-of-function allele behaves as above, but the single standard allele in the heterozygous genotype produces sufficient gene product to produce the same, standard phenotype as seen in the homozygote. Haplosufficiency accounts for the typical dominance of the “standard” allele over variant alleles, where the phenotypic identity of genotypes heterozygous and homozygous for the allele defines it as dominant, versus a variant phenotype produced by only by the genotype homozygous for the alternative allele, which defines it as recessive. The systems could also be used to induce a co-delivered gene not normally found in the target cells, for example, a cancer killing protein.

The following table provides examples of genes for which haploinsufficiency can lead to disease:

TABLE 1
Genes Associated With Haploinsufficiency Diseases

| | Entrez | Chro- | |
|---|---|---|---|
| Gene | Gene | mo- | |
| Symbol | ID | some | Disorder/Syndrome |
| TP73 | 7161 | 1 | prostate hyperplasia and |
| | | | prostate cancer |
| DFFB | 1677 | 1 | oligodendroglioma development |
| KCNAB2 | 8514 | 1 | characteristic craniofacial |
| | | | abnormalities, mental retardation, |
| | | | and epilepsy with 1p36 deletion |
| | | | syndrome |
| CHD5 | 26038 | 1 | monosomy 1p36 syndrome |
| CAMTA1 | 23261 | 1 | tumors development |
| PINK1 | 65018 | 1 | sporadic early-onset parkinsonism |
| SAM68 | 10657 | 1 | mammary tumor onset and |
| | | | tumor multiplicity |
| KCNQ4 | 9132 | 1 | DEAFNESS, AUTOSOMAL |
| | | | DOMINANT NONSYNDROMIC |
| | | | SENSORINEURAL 2 |
| GLUT1 | 6513 | 1 | Facilitated glucose transporter |
| | | | protein type 1 (GLUT1) |
| | | | deficiency syndrome |
| MYH | 4595 | 1 | hepatocellular carcinoma and |
| | | | cholangiocarcinom |
| FOXE3 | 2301 | 1 | anterior segment dysgenesis |
| | | | similar to Peters' anomaly |
| HUD | 1996 | 1 | poor prognosis |
| INK4C | 1031 | 1 | medulloblastoma formation |
| NFIA | 4774 | 1 | Complex central nervous system |
| | | | (CNS) malformations and urinary |
| | | | tract defects |
| CCN1 | 3491 | 1 | delayed formation of the ventricular |
| | | | septum in the embryo and persistent |
| | | | ostium primum atrial septal defects |
| ABCA4 | 24 | 1 | Stargardt disease, retinitis |
| | | | pigmentosa-19, and macular |
| | | | degeneration age-related 2 |
| WNT2B | 7482 | 1 | mental retardation, short |
| | | | stature and colobomata |
| ADAR | 103 | 1 | dyschromatosis symmetrica |
| | | | hereditaria |
| ATP1A2 | 477 | 1 | familial hemiplegic migraine type 2 |
| MPZ | 4359 | 1 | neurologic diseases, including |
| | | | CHN, DSS, and CMT1B |
| MYOC | 4653 | 1 | hereditary juvenile-onset |
| | | | open-angle glaucoma |
| HRPT2 | 79577 | 1 | Ossifying fibroma (progressive |
| | | | enlargement of the affected jaw) |
| LRH-1 | 2494 | 1 | inflammatory bowel disease |
| IRF6 | 3664 | 1 | van der Woude syndrome and |
| | | | popliteal pterygium syndrome |
| PROX1 | 5629 | 1 | Lymphatic vascular defects, |
| | | | adult-onset obesity |
| TP53BP2 | 7159 | 1 | no suppression of tumor growth |
| NLRP3 | 114548 | 1 | CINCA syndrome |
| ID2 | 3398 | 2 | Congenital hydronephrosis |
| MYCN | 4613 | 2 | reduced brain size and intestinal |
| | | | atresias in Feingold syndrome |
| GCKR | 2646 | 2 | one form of maturity onset diabetes |
| | | | of the young |
| SPAST | 6683 | 2 | SPASTIC PARAPLEGIA 4 |
| MSH6 | 2956 | 2 | limitation of mismatch repair |
| FSHR | 2492 | 2 | degenerative changes in the central |
| | | | nervous system |
| SPR | 6697 | 2 | dopa-responsive dystonia |
| PAX8 | 7849 | 2 | congenital hypothyroidism |
| SMADIP1 | 9839 | 2 | syndromic Hirschsprung disease |
| RPRM | 56475 | 2 | tumorigenesis, no suppression |
| | | | of tumor growth |
| SCN1A | 6323 | 2 | Severe myoclonic epilepsy of |
| | | | infancy (SMEI) or Dravet syndrome |
| HOXD13 | 3239 | 2 | foot malformations |
| COL3A1 | 1281 | 2 | Ehlers-Danlos syndrome type |
| | | | IV, and with aortic and arterial |
| | | | aneurysms |
| SLC40A1 | 30061 | 2 | ferroportin disease |
| SATB2 | 23314 | 2 | craniofacial dysmorphologies, |
| | | | cleft palate |
| SUMO1 | 7341 | 2 | nonsyndromic cleft lip and palate |
| BMPR2 | 659 | 2 | primary pulmonary hypertension |
| XRCC5 | 7520 | 2 | retarded growth, increased |
| | | | radiosensitivity, elevated p53 |
| | | | levels and shortened telomeres |
| PAX3 | 5077 | 2 | developmental delay and autism |
| STK25 | 10494 | 2 | mild-to-moderate mental retardation |
| | | | with an Albright hereditary |
| | | | osteodystrophy-like phenotype |
| CHL1 | 10752 | 3 | 3p deletion (3p−) syndrome |
| SRGAP3 | 9901 | 3 | severe mental retardation |
| VHL | 7428 | 3 | increased lung cancer susceptibility |
| GHRL | 51738 | 3 | GHRELIN POLYMORPHISM |
| PPARG | 5468 | 3 | susceptibility to mammary, ovarian |
| | | | and skin carcinogenesis |
| SRG3 | 6599 | 3 | proteasomal degradation |
| RASSF1A | 11186 | 3 | pathogenesis of a variety of cancers, |
| | | | no suppression of tumor growth |
| TKT | 7086 | 3 | reduced adipose tissue and |
| | | | female fertility |
| MITF | 4286 | 3 | Waardenburg syndrome type 2 |
| FOXP1 | 27086 | 3 | tumors development |
| ROBO1 | 6091 | 3 | predispose to dyslexia |
| DIRC2 | 84925 | 3 | onset of tumor growth |
| ATP2C1 | 27032 | 3 | orthodisease, skin disorder |
| FOXL2 | 668 | 3 | blepharophimosis syndrome |
| | | | associated with ovarian dysfunction |
| ATR | 545 | 3 | mismatch repair-deficient |
| SI | 6476 | 3 | SUCRASE-ISOMALTASE |
| | | | DEFICIENCY, CONGENITAL |
| TERC | 7012 | 3 | Autosomal dominant dyskeratosis |
| | | | congenita (ADDC), a rare inherited |
| | | | bone marrow failure syndrome |
| SOX2 | 6657 | 3 | hippocampal malformations |
| | | | and epilepsy |
| OPA1 | 4976 | 3 | optic atrophy |
| TFRC | 7037 | 3 | stressed erythropoiesis and |
| | | | neurologic abnormalities |
| FGFR3 | 2261 | 4 | a variety of skeletal dysplasias, |
| | | | including the most common genetic |
| | | | form of dwarfism, achondroplasia |
| LETM1 | 3954 | 4 | Wolf Hirshhorn syndrome |
| SH3BP2 | 6452 | 4 | Wolf-Hirschhorn syndrome |
| MSX1 | 4487 | 4 | oligodontia |
| RBPJ | 3516 | 4 | embryonic lethality and formation |
| | | | of arteriovenous malformations |
| PHOX2B | 8929 | 4 | predispose to Hirschsprung disease |
| ENAM | 10117 | 4 | Amelogenesis imperfecta (inherited |
| | | | defects of dental enamel formation) |
| MAPK10 | 5602 | 4 | epileptic encephalopathy of the |
| | | | Lennox-Gaustaut type |
| PKD2 | 5311 | 4 | Autosomal dominant polycystic |
| | | | kidney disease |
| SNCA | 6622 | 4 | familial Parkinson's disease |
| RIEG | 5308 | 4 | Rieger syndrome (RIEG) |
| | | | characterized by malformations |
| | | | of the anterior segment of the eye, |
| | | | failure of the periumbilical skin to |
| | | | involute, and dental hypoplasia |
| ANK2 | 287 | 4 | arrhythmia |
| MAD2L1 | 4085 | 4 | optimal hematopoiesis |
| PLK4 | 10733 | 4 | mitotic infidelity and carcinogenesis |
| FBXW7 | 55294 | 4 | cancer (breast, ovary) tumors |
| | | | development |
| TERT | 7015 | 5 | DYSKERATOSIS CONGENITA |
| SEMA5A | 9037 | 5 | abnormal brain development |
| GDNF | 2668 | 5 | complex human diseases |
| | | | (Hirschsprung-like intestinal |
| | | | obstruction and early-onset lethality) |
| FGF10 | 2255 | 5 | craniofacial development and |
| | | | developmental disorders |
| PIK3R1 | 5295 | 5 | insulin resistance |
| APC | 324 | 5 | familial adenomatous polyposis |
| RAD50 | 10111 | 5 | hereditary breast cancer |
| | | | susceptibility associated with |
| | | | genomic instability |
| SMAD5 | 4090 | 5 | secondary myelodysplasias |
| | | | and acute myeloid leukemias |
| EGR1 | 1958 | 5 | development of myeloid disorders |
| TCOF1 | 6949 | 5 | depletion of neural crest cell |
| | | | precursors, Treacher Collins |
| | | | syndrome |
| NPM1 | 4869 | 5 | myelodysplasias and leukemias |
| NKX2-5 | 1482 | 5 | microcephaly and congenital |
| | | | heart disease |
| MSX2 | 4488 | 5 | pleiotropic defects in bone growth |
| | | | and ectodermal organ formation |
| NSD1 | 64324 | 5 | Sotos syndrome |
| FOXC1 | 2296 | 6 | Axenfeld-Rieger anomaly of |
| | | | the anterior eye chamber |
| DSP | 1832 | 6 | skin fragility/woolly hair syndrome; |
| | | | disruption of tissue structure, |
| | | | integrity and changes in |
| | | | keratinocyte proliferation |
| EEF1E1 | 9521 | 6 | no suppression of tumor growth |
| TNXA | 7146 | 6 | Ehlers-Danlos syndrome |
| TNX | 7148 | 6 | Elastic fiber abnormalities in |
| | | | hypermobility type |
| | | | Ehlers-Danlos syndrome |
| HMGA1 | 3159 | 6 | insulin resistance and diabetes |
| RUNX2 | 860 | 6 | cleidocranial dysplasia |
| CD2AP | 23607 | 6 | glomerular disease susceptibility |
| ELOVL4 | 6785 | 6 | defective skin permeability barrier |
| | | | function and neonatal lethality |
| NT5E | 4907 | 6 | Neuropathy target esterase |
| | | | deficiency |
| SIM1 | 6492 | 6 | impaired melanocortin-mediated |
| | | | anorexia and activation of |
| | | | paraventricular nucleus neurons |
| COL10A1 | 1300 | 6 | Schmid type metaphyseal |
| | | | chondrodysplasia and Japanese |
| | | | type spondylometaphyseal dysplasia |
| PARK2 | 5071 | 6 | PARKINSON DISEASE 2 |
| TWIST1 | 7291 | 7 | coronal synostosis |
| GLI3 | 2737 | 7 | Greig cephalopolysyndactyly |
| | | | and Pallister-Hall syndromes |
| GCK | 2645 | 7 | non-insulin dependent diabetes |
| | | | mellitus (NIDDM), maturity onset |
| | | | diabetes of the young, type 2 |
| | | | (MODY2) and persistent |
| | | | hyperinsulinemic hypoglycemia of |
| | | | infancy (PHHI) |
| FKBP6 | 8468 | 7 | Williams-Beuren syndrome |
| ELN | 2006 | 7 | cardiovascular disease and |
| | | | connective tissue abnormalities |
| LIMK1 | 3984 | 7 | Williams syndrome (WS), a |
| | | | neurodevelopmental disorder |
| RFC2 | 5982 | 7 | growth deficiency as well as |
| | | | developmental disturbances in |
| | | | Williams syndrome |
| GTF3 | 9569 | 7 | abnormal muscle fatiguability |
| GTF2I | 2969 | 7 | Williams-Beuren syndrome |
| NCF1 | 653361 | 7 | autosomal recessive chronic |
| | | | granulomatous disease |
| KRIT1 | 889 | 7 | Cerebral Cavernous Malformations |
| | | | (vascular malformations |
| | | | characterised by abnormally |
| | | | enlarged capillary cavities) |
| COL1A2 | 1278 | 7 | subtle symptoms like recurrent |
| | | | joint subluxation or hypodontia |
| SHFM1 | 7979 | 7 | severe mental retardation, short |
| | | | stature, microcephaly and deafness |
| RELN | 5649 | 7 | Cognitive disruption and altered |
| | | | hippocampus synaptic function |
| FOXP2 | 93986 | 7 | Speech and language impairment |
| | | | and oromotor dysprax |
| CAV1 | 857 | 7 | 17beta-estradiol-stimulated |
| | | | mammary tumorigenesis |
| ST7 | 7982 | 7 | no suppression of tumor growth |
| BRAF | 673 | 7 | Cardiofaciocutaneous (CFC) |
| | | | syndrome |
| SHH | 6469 | 7 | Holoprosencephaly, sacral |
| | | | anomalies, and situs ambiguus |
| HLXB9 | 3110 | 7 | Currarino syndrome including a |
| | | | presacral mass, sacral agenesis, and |
| | | | anorectal malformation |
| GATA4 | 2626 | 8 | congenital heart disease |
| NKX3-1 | 4824 | 8 | prostate cancer |
| FGFR1 | 2260 | 8 | Pfeiffer syndrome, Jackson-Weiss |
| | | | syndrome, Antley-Bixler |
| | | | syndrome, osteoglophonic |
| | | | dysplasia, and autosomal dominant |
| | | | Kallmann syndrome 2 |
| CHD7 | 55636 | 8 | CHARGE syndrome |
| CSN5 | 10987 | 8 | TRC8 hereditary kidney cancer |
| EYA1 | 2138 | 8 | branchiootorenal dysplasia |
| | | | syndrome, branchiootic syndrome, |
| | | | and sporadic cases of congenital |
| | | | cataracts and ocular anterior |
| | | | segment anomalies |
| TRPS1 | 7227 | 8 | dominantly inherited |
| | | | tricho-rhino-phalangeal (TRP) |
| | | | syndromes |
| DMRT1 | 1761 | 9 | failure of testicular development |
| | | | and feminization in male |
| DMRT2 | 10655 | 9 | defective testis formation in |
| | | | karyotypic males and impaired |
| | | | ovary function in karyotypic females |
| MLLT3 | 4300 | 9 | neuromotor developmental delay, |
| | | | cerebrellar ataxia, and epilepsy |
| ARF | 1029 | 9 | acute myeloid leukemia |
| CDKN2B | 1030 | 9 | syndrome of cutaneous malignant |
| | | | melanoma and nervous system |
| | | | tumors |
| BAG1 | 573 | 9 | lung tumorigenesis |
| PAX5 | 5079 | 9 | pathogensis of lymphocytic |
| | | | lymphomas |
| GCNT1 | 2650 | 9 | T lymphoma cells resistant to |
| | | | cell death |
| ROR2 | 4920 | 9 | basal cell nevus symdrome (BCNS) |
| PTCH1 | 5727 | 9 | Primitive neuroectodermal |
| | | | tumors formation |
| NR5A1 | 2516 | 9 | impaired testicular development, |
| | | | sex reversal, and adrenal failure |
| LMX1B | 4010 | 9 | nail-patella syndrome |
| ENG | 2022 | 9 | Hereditary hemorrhagic |
| | | | telangiectasia type 1 |
| TSC1 | 7248 | 9 | transitional cell carcinoma of |
| | | | the bladder |
| COL5A1 | 1289 | 9 | Structural abnormalities of the |
| | | | cornea and lid |
| NOTCH1 | 4851 | 9 | aortic valve disease (cardiac |
| | | | malformation and aortic valve |
| | | | calcification) |
| EHMT1 | 79813 | 9 | 9q34 subtelomeric deletion |
| | | | syndrome |
| KLF6 | 1316 | 10 | cellular growth dysregulation |
| | | | and tumorigenesis |
| GATA3 | 2625 | 10 | HDR (hypoparathyroidism, |
| | | | deafness and renal dysplasia) |
| | | | syndrome |
| ANX7 | 310 | 10 | tumorigenesis |
| PTEN | 5728 | 10 | prostate cancer high-grade prostatic |
| | | | intra-epithelial neoplasias |
| PAX2 | 5076 | 10 | renal-coloboma syndrome |
| FGF8 | 2253 | 10 | several human craniofacial disorders |
| BUB3 | 9184 | 10 | short life span that is associated |
| | | | with the early onset of |
| | | | aging-related features |
| CDKN1C | 1028 | 11 | Beckwith-Wiedemann syndrome |
| NUP98 | 4928 | 11 | destruction of securin in mitosis |
| PAX6 | 5080 | 11 | eye diseases |
| WT1 | 7490 | 11 | congenital genitourinary (GU) |
| | | | anomalies and/or bilateral |
| | | | disease and tumorigenesis |
| EXT2 | 2132 | 11 | type II form of multiple exostoses |
| ALX4 | 60529 | 11 | Tibialaplasia, lower extremity |
| | | | mirror image polydactyly, |
| | | | brachyphalangy, craniofacial |
| | | | dysmorphism and genital hypoplasia |
| FEN1 | 2237 | 11 | neuromuscular and |
| | | | neurodegenerative diseases |
| SF1 | 7536 | 11 | mild gonadal dysgenesis and |
| | | | impaired androgenization |
| FGF3 | 2248 | 11 | otodental syndrome |
| FZD4 | 8322 | 11 | complex chromosome rearrangement |
| | | | with multiple abnormalities |
| | | | including growth retardation, facial |
| | | | anomalies, exudative |
| | | | vitreoretinopathy (EVR), cleft |
| | | | palate, and minor digital anomalies |
| ATM | 472 | 11 | High incidence of cancer |
| H2AX | 3014 | 11 | genomic instability, early onset |
| | | | of various tumors |
| FLI1 | 2313 | 11 | Paris-Trousseau thrombopenia |
| NFRKB | 4798 | 11 | cellular immunodeficiency, |
| | | | pancytopenia, malformations |
| PHB2 | 11331 | 12 | enhanced estrogen receptor function |
| ETV6 | 2120 | 12 | a pediatric pre-B acute |
| | | | lymphoblastic leukemia |
| CDKN1B | 1027 | 12 | ErbB2-induced mammary |
| | | | tumor growth |
| COL2A1 | 1280 | 12 | Stickler syndrome |
| KRT5 | 3852 | 12 | epidermolysis bullosa simplex |
| MYF6 | 4618 | 12 | myopathy and severe course of |
| | | | Becker muscular dystrophy |
| IGF1 | 3479 | 12 | subtle inhibition of intrauterine |
| | | | and postnatal growth |
| SERCA2 | 488 | 12 | colon and lung cancer |
| TBX5 | 6910 | 12 | maturation failure of conduction |
| | | | system morphology and function in |
| | | | Holt-Oram syndrome |
| TBX3 | 6926 | 12 | ulnar-mammary syndrome |
| HNF1A | 6927 | 12 | reduced serum apolipoprotein |
| | | | M levels |
| BRCA2 | 675 | 13 | predisposed to breast, ovarian |
| | | | pancreatic and other cancers |
| FKHR | 2308 | 13 | Alveolar rhabdomyosarcomas |
| RB1 | 5925 | 13 | Metaphase cytogenetic abnormalities |
| ZIC2 | 7546 | 13 | neurological disorders, behavioral |
| | | | abnormalities |
| LIG4 | 3981 | 13 | LIG4 syndrome, nonlymphoid |
| | | | tumorigenesis |
| COCH | 1690 | 14 | unknown |
| NPAS3 | 64067 | 14 | schizophrenia |
| NKX2-1 | 7080 | 14 | Choreoathetosis, hypothyroidism, |
| | | | pulmonary alterations, neurologic |
| | | | phenotype and secondary |
| | | | hyperthyrotropinemia, and diseases |
| | | | due to transcription factor defects |
| PAX9 | 5083 | 14 | posterior tooth agenesis |
| BMP4 | 652 | 14 | a contiguous gene syndrome |
| | | | comprising anophthalmia, pituitary |
| | | | hypoplasia, and ear anomalies |
| GCH1 | 2643 | 14 | malignant hyperphenylalaninemia |
| | | | and dopa-responsive dystonia |
| SIX6 | 4990 | 14 | bilateral anophthalmia and |
| | | | pituitary anomalies |
| RAD51B | 5890 | 14 | centrosome fragmentation and |
| | | | aneuploidy |
| BCL11B | 64919 | 14 | suppression of |
| | | | lymphomagenesis and thymocyte |
| | | | development |
| SPRED1 | 161742 | 15 | neurofibromatosis type 1-like |
| | | | syndrome |
| BUBR1 | 701 | 15 | enhanced tumor development |
| DLL4 | 54567 | 15 | embryonic lethality due to |
| | | | major defects in arterial and |
| | | | vascular development |
| FBN1 | 2200 | 15 | Marfan syndrome, isolated ectopia |
| | | | lentis, autosomal dominant |
| | | | Weill-Marchesani syndrome, MASS |
| | | | syndrome, and Shprintzen-Goldberg |
| | | | craniosynostosis syndrome |
| ALDH1A2 | 8854 | 15 | facilitate posterior organ |
| | | | development and prevent spina |
| | | | bifida |
| TPM1 | 7168 | 15 | type 3 familial hypertrophic |
| | | | cardiomyopathy |
| P450SCC | 1583 | 15 | 46, XY sex reversal and adrenal |
| | | | insufficiency |
| BLM | 641 | 15 | the autosomal recessive |
| | | | disorder Bloom syndrome |
| COUP-TFII | 7026 | 15 | several malformations, pre- and |
| | | | postnatal growth |
| | | | retardation and developmental |
| SOX8 | 30812 | 16 | the mental retardation found in |
| | | | ATR-16 syndrome |
| TSC2 | 7249 | 16 | differential cancer susceptibility |
| PKD1 | 5310 | 16 | autosomal dominant polycystic |
| | | | kidney disease |
| CBP | 1387 | 16 | Rubinstein-Taybi syndrome |
| SOCS1 | 8651 | 16 | sever liver fibrosis and |
| | | | hepatitis-induced carcinogenesis |
| PRM2 | 5620 | 16 | infertility |
| PRM1 | 5619 | 16 | infertility |
| ABCC6 | 368 | 16 | pseudoxanthoma elasticum |
| ERAF | 51327 | 16 | subtle erythroid phenotype |
| SALL1 | 6299 | 16 | Townes-Brocks syndrome |
| CBFB | 865 | 16 | delayed cranial ossification, cleft |
| | | | palate, congenital heart anomalies, |
| | | | and feeding difficulties |
| CTCF | 10664 | 16 | loss of imprinting of insulin-like |
| | | | growth factor-II in Wilms tumor |
| WWOX | 51741 | 16 | initiation of tumor development |
| FOXF1 | 2294 | 16 | defects in formation and branching |
| | | | of primary lung buds |
| FOXC2 | 2303 | 16 | the lymphatic/ocular disorder |
| | | | Lymphedema-Distichiasis |
| YWHAE | 7531 | 17 | pathogenesis of small cell lung |
| | | | cancer |
| HIC1 | 3090 | 17 | Miller-Dieker syndrome |
| LIS1 | 5048 | 17 | abnormal cell proliferation, |
| | | | migration and differentiation in |
| | | | the adult dentate gyrus |
| P53 | 7157 | 17 | male oral squamous cell carcinomas |
| PMP22 | 5376 | 17 | hereditary neuropathy with |
| | | | liability to pressure palsies |
| COPS3 | 8533 | 17 | Circadian rhythm |
| | | | abnormalities of melatonin in |
| | | | Smith-Magenis syndrome |
| RAI1 | 10743 | 17 | Smith-Magenis syndrome |
| TOP3A | 7156 | 17 | Smith-Magenis syndrome |
| SHMT1 | 6470 | 17 | Smith-Magenis syndrome |
| RNF135 | 84282 | 17 | phenotypic abnormalities |
| | | | including overgrowth |
| NF1 | 4763 | 17 | neurofibromatosis type 1 |
| SUZ12 | 23512 | 17 | mental impairment in constitutional |
| | | | NF1 microdeletions |
| MEL-18 | 7703 | 17 | breast carcinogenesis |
| KLHL10 | 317719 | 17 | disrupted spermiogenesis |
| STAT5B | 6777 | 17 | striking amelioration of |
| | | | IL-7-induced mortality and |
| | | | disease development |
| STAT5A | 6776 | 17 | striking amelioration of |
| | | | IL-7-induced mortality and |
| | | | disease development |
| BECN1 | 8678 | 17 | autophagy function, and tumor |
| | | | suppressor function |
| BRCA1 | 672 | 17 | shortened life span and |
| | | | ovarian tumorigenesis |
| PGRN | 2896 | 17 | neurodegeneration |
| MAPT | 4137 | 17 | neuronal cell death, |
| | | | neurodegenerative disorders such as |
| | | | Alzheimer's disease, Pick's disease, |
| | | | frontotemporal dementia, |
| | | | cortico-basal degeneration and |
| | | | progressive supranuclear palsy |
| CSH1 | 1442 | 17 | Silver-Russell syndrome |
| POLG2 | 11232 | 17 | mtDNA deletions causes COX |
| | | | deficiency in muscle fibers and |
| | | | results in the clinical phenotype |
| PRKAR1A | 5573 | 17 | Carney complex, a familial |
| | | | multiple neoplasia syndrome |
| SOX9 | 6662 | 17 | skeletal dysplasia |
| NHERF1 | 9368 | 17 | breast tumors |
| FSCN2 | 25794 | 17 | photoreceptor degeneration, |
| | | | autosomal dominant retinitis |
| | | | pigmentosa |
| DSG1 | 1828 | 18 | diseases of epidermal integrity |
| DSG2 | 1829 | 18 | ARRHYTHMOGENIC RIGHT |
| | | | VENTRICULAR DYSPLASIA |
| TCF4 | 6925 | 18 | Pitt-Hopkins syndrome, a |
| | | | syndromic mental disorder |
| FECH | 2235 | 18 | protoporphyria |
| MC4R | 4160 | 18 | increased adiposity and linear |
| | | | growth |
| GALR1 | 2587 | 18 | uncontrolled proliferation and |
| | | | neoplastic transformation |
| SALL3 | 27164 | 18 | 18q deletion syndrome |
| LKB1 | 6794 | 19 | Peutz-Jeghers syndrome |
| PNPLA6 | 10908 | 19 | organophosphorus-induced |
| | | | hyperactivity and toxicity |
| RYR1 | 6261 | 19 | malignant hyperthermia |
| | | | susceptibility, central core disease, |
| | | | and minicore myopathy with |
| | | | external ophthalmoplegia |
| TGFB1 | 7040 | 19 | Aggressive pancreatic ductal |
| | | | adenocarcinoma |
| RPS19 | 6223 | 19 | Diamond-Blackfan anemia |
| DMPK | 1760 | 19 | cardiac disease in myotonic |
| | | | dystrophy |
| CRX | 1406 | 19 | photoreceptor degeneration, |
| | | | Leber congenital amaurosis |
| | | | type III and the autosomal |
| | | | dominant cone-rod dystrophy 2 |
| PRPF31 | 26121 | 19 | retinitis pigmentosa with |
| | | | reduced penetrance |
| JAG1 | 182 | 20 | Alagille syndrome |
| PAX1 | 5075 | 20 | Klippel-Feil syndrome |
| GDF5 | 8200 | 20 | Multiple-synostosis syndrome |
| HNF4A | 3172 | 20 | monogenic autosomal dominant |
| | | | non-insulin-dependent |
| | | | diabetes mellitus type I |
| SALL4 | 57167 | 20 | Okihiro syndrome |
| MC3R | 4159 | 20 | susceptibility to obesity |
| RAE1 | 8480 | 20 | premature separation of sister |
| | | | chromatids, severe aneuploidy |
| | | | and untimely degradation of securin |
| GNAS | 2778 | 20 | reduced activation of a downstream |
| | | | target in epithelial tissues |
| EDN3 | 1908 | 20 | Hirschsprung disease |
| KCNQ2 | 3785 | 20 | epilepsy susceptibility |
| SOX18 | 54345 | 20 | mental retardation |
| SLC5A3 | 6526 | 21 | brain inositol deficiency |
| RUNX1 | 861 | 21 | The 8p11 myeloproliferative |
| | | | syndrome |
| DYRK1A | 1859 | 21 | neurological defects, |
| | | | developmental delay |
| COL6A1 | 1291 | 21 | autosomal dominant disorder, |
| | | | Bethlem myopathy |
| PRODH | 5625 | 22 | 22q11 Deletion syndrome |
| DGCR2 | 9993 | 22 | DiGeorge syndrome |
| HIRA | 7290 | 22 | DiGeorge syndrome (craniofacial, |
| | | | cardiac and thymic malformations) |
| TBX1 | 6899 | 22 | 22q11 deletion syndrome and |
| | | | schizophrenia |
| COMT | 1312 | 22 | 22q11.2 deletion syndrome |
| RTN4R | 65078 | 22 | schizophrenia susceptibility |
| | | | (schizoaffective disorders are |
| | | | common features in patients |
| | | | with DiGeorge/velocardiofacial |
| | | | syndrome) |
| PCQAP | 51586 | 22 | DiGeorge syndrome |
| LZTR1 | 8216 | 22 | DiGeorge syndrome |
| INI1 | 6598 | 22 | pituitary tumorigenesis |
| MYH9 | 4627 | 22 | hematological abnormalities |
| SOX10 | 6663 | 22 | the etiology of |
| | | | Waardenburg/Hirschsprung disease |
| FBLN1 | 2192 | 22 | limb malformations |
| PPARA | 5465 | 22 | prostate cancer |
| PROSAP2 | 85358 | 22 | the terminal 22q13.3 deletion |
| | | | syndrome, characterized by severe |
| | | | expressive-language delay, mild |
| | | | mental retardation, hypotonia, joint |
| | | | laxity, dolichocephaly, and minor |
| | | | facial dysmorphisms |
| SHOX | 6473 | X | congenital form of growth failure, |
| | | | the aetiology of “idiopathic” short |
| | | | stature and the growth deficits and |
| | | | skeletal anomalies in Leri Weill, |
| | | | Langer and Turner syndrome |
| P2RY8 | 286530 | X | mentally retarded males |
| NLGN4X | 57502 | X | autism and Asperger syndrome |
| TRAPPC2 | 6399 | X | spondyloepiphyseal dysplasia tarda |
| RPS4X | 6191 | X | unknown |
| CSF2RA | 1438 | X | growth deficiency |
| CHRDL1 | 91851 | X | topographic retinotectal projection |
| | | | and in the regulation of retinal |
| | | | andiogenesis in response to hypoxia |
| SF3B4 | 10262 | 1 | Nager syndrome, Hepatocellular |
| | | | carcinoma and Rodriguez |
| | | | Acrofacial Dysotosis |
| CTNND2 | 1501 | 5 | Intellectual disability, epilepsy |
| AAGAB | 79719 | 15 | Buschke-Fischer-Brauer and |
| | | | punctuate palmoplantar keratoderma |
| ABCD1 | 215 | X | adrenoleukodystrophy |
| AKT3 | 10000 | 1 | Developmental disorders and |
| | | | breast cancer |
| ANKRD11 | 29123 | 16 | KBG syndrome |
| ANOS1 | 3730 | X | Kallman syndrome |
| AP1S2 | 8905 | X | Mental retardation |
| AR | 367 | X | Kennedy's disease and |
| | | | androgen insensitivity |
| ARSE | 415 | X | chondrodysplasia punctata |
| ARX | 170302 | X | cognitive disability and epilepsy |
| ASXL1 | 171023 | 20 | myelodysplastic syndromes and |
| | | | chronic myelomonocytic leukemia |
| ATP7A | 538 | X | Menkes disease, X-linked distal |
| | | | spinal muscular atrophy, and |
| | | | occipital horn syndrome |
| ATP8A2 | 51761 | 13 | cerebellar ataxia and cognitive |
| | | | disabilities |
| ATRX | 546 | X | cognitive disabilities as well as |
| | | | alpha-thalassemia (ATRX) |
| | | | syndrome |
| AUTS2 | 26053 | 7 | autism spectrum disorders, |
| | | | intellectual disability, and |
| | | | developmental delay |
| AVPR2 | 554 | X | Nephrogenic Diabetes Insipidus (NDI) |
| BAG3 | 9531 | 10 | cardiomyopathy |
| BCL11A | 53335 | 2 | Autism and intellectual development |
| BCOR | 54880 | X | sarcoma of the kidney |
| BMPR1A | 657 | 10 | Intellectual disability |
| BRWD3 | 254065 | X | cognitive disabilities and |
| | | | X-linked macrocephaly |
| BTK | 695 | X | agammaglobulinemia |
| CACNA1C | 775 | 12 | Autism |
| CASK | 8573 | X | FG syndrome 4, intellectual |
| | | | disability and microcephaly |
| CDH1 | 999 | 16 | breast, colorectal, thyroid, |
| | | | gastric and ovarian cancer |
| CDKL5 | 6792 | X | infantile spasm syndrome (ISSX), |
| | | | also known as X-linked West |
| | | | syndrome, and Rett syndrome (RTT). |
| CHD2 | 1106 | 15 | Neurodevelopmental disorders |
| CHD8 | 57680 | 14 | Autism |
| CHM | 1121 | X | choroideremia |
| CHRM3 | 1131 | 1 | Schizophrenia |
| CLCN5 | 1184 | X | Dent disease and renal tubular |
| | | | disorders complicated by |
| | | | nephrolithiasis |
| CNKSR2 | 22866 | X | Intellectual disability |
| CNTN4 | 152330 | 3 | autism spectrum disorders |
| CNTNAP2 | 26047 | 7 | neurodevelopmental disorders, |
| | | | including Gilles de la Tourette |
| | | | syndrome, schizophrenia, |
| | | | epilepsy, autism, ADHD and |
| | | | intellectual disability |
| COL11A1 | 1301 | 1 | Fibrochondrogenesis, Stickler |
| | | | syndrome and with Marshall |
| | | | syndrome |
| COL1A1 | 1277 | 17 | imperfecta types I-IV, Ehlers-Danlos |
| | | | syndrome type VIIA, Ehlers-Danlos |
| | | | syndrome Classical type, Caffey |
| | | | Disease and idiopathic osteoporosis |
| CREBBP | 1387 | 16 | Rubinstein-Taybi syndrome (RTS) |
| | | | and acute myeloid leukemia |
| CRYBB2 | 1415 | 22 | Cataracts and prostate cancer |
| CUL4B | 8450 | X | Intellectual disability |
| CYBB | 1536 | X | chronic granulomatous disease |
| | | | (CGD |
| DCX | 1641 | X | pilepsy, cognitive disability, |
| | | | subcortical band heterotopia |
| | | | and lissencephaly syndrome |
| DICER1 | 23405 | 14 | familial tumor susceptibility |
| | | | syndrome |
| DKC1 | 1736 | X | X-linked dyskeratosis congenita |
| DLG3 | 1741 | X | cognitive disability |
| DMD | 1756 | X | uchenne muscular dystrophy (DMD), |
| | | | Becker muscular dystrophy (BMD), |
| | | | and cardiomyopathy |
| DSC2 | 1824 | 18 | arrhythmogenic right ventricular |
| | | | dysplasia-11, and cancer |
| EBP | 10682 | X | Chondrodysplasia punctata 2 |
| EDNRB | 1910 | 13 | Hirschsprung disease type 2 |
| EDA | 1896 | X | X-linked hypohidrotic |
| | | | ectodermal dysplasia |
| EFNB1 | 1947 | X | craniofrontonasal syndrome |
| EFTUD2 | 9343 | 17 | mandibulofacial dysostosis |
| | | | with microcephaly |
| EMX2 | 2018 | 10 | schizencephaly |
| EP300 | 2033 | 22 | Rubinstein-Taybi syndrome |
| | | | and epithelial cancer |
| ERF | 2077 | 19 | craniosynostosis |
| ERMARD | 55780 | 6 | Periventricular nodular heterotopia |
| EXT1 | 2131 | 8 | Multiple osteochondromas |
| EYA4 | 2070 | 6 | Cardiomyopathy and hearing loss |
| F8 | 2157 | X | hemophilia A |
| F9 | 2158 | X | hemophilia B or Christmas disease |
| FAM58A | 92002 | X | STAR syndrome |
| FANCB | 2187 | X | VACTERL syndrome |
| FAS | 355 | 10 | Autoimmune lymphoproliferative |
| | | | syndrome |
| FGD1 | 2245 | X | dysplasia in Aarskog-Scott |
| | | | syndrome and a syndromatic form |
| | | | of X-linked cognitive disability |
| FLCN | 201163 | 17 | Birt-Hogg-Dube syndrome |
| FLG | 2312 | 1 | ichthyosis vulgaris |
| FLNA | 2316 | X | Periventricular nodular heterotopias, |
| | | | otopalatodigital syndromes, |
| | | | frontometaphyseal dysplasia, |
| | | | Melnick-Needles syndrome, and |
| | | | X-linked congenital idiopathic |
| | | | intestinal pseudoobstruction |
| FOXG1 | 2290 | 14 | Rett syndrome |
| FRMD7 | 90167 | X | congenital nystagmus |
| FTSJ1 | 24140 | X | cognitive disability |
| GATA2 | 2624 | 3 | monocytopenia and mycobacterial |
| | | | infection syndrome and Emberger |
| | | | syndrome |
| GATA6 | 2627 | 18 | congenital defects and |
| | | | cardiomyopathy |
| GDI1 | 2664 | X | cognitive disability |
| GJA5 | 2702 | 1 | atrial fibrillation |
| GJA8 | 2703 | 1 | zonular pulverulent cataracts, nuclear |
| | | | progressive cataracts, and |
| | | | cataract-microcornea syndrome |
| GK | 2710 | X | glycerol kinase deficiency |
| GLA | 2717 | X | Fabry disease |
| GLI2 | 2736 | 2 | Greig cephalopolysyndactyly |
| | | | syndrome, Pallister-Hall syndrome, |
| | | | preaxial polydactyly type IV, |
| | | | postaxial polydactyly types A1 and B |
| GLMN | 11146 | 1 | glomuvenous malformations |
| GPC3 | 2719 | X | Simpson-Golabi-Behmel syndrome |
| GRIA3 | 2892 | X | Intellectual disability |
| GRIN2A | 2903 | 16 | epilepsy and speech disorder |
| GRIN2B | 2904 | 12 | neurodevelopmental disorders |
| | | | autism, attention deficit hyperactivity |
| | | | disorder, epilepsy and schizophrenia |
| HCCS | 3052 | X | microphthalmia syndrome |
| HDAC4 | 9759 | 2 | Mental retardation |
| HMGA2 | 8091 | 12 | Silver-Russell syndrome |
| HNF1B | 6928 | 17 | Intellectual disability |
| HNRNPK | 3190 | 9 | Intellectual disability |
| HPRT1 | 3251 | X | Lesch-Nyhan syndrome or gout |
| HNRNPU | 3192 | 1 | epileptic encephalopathy and |
| | | | intellectual disability |
| IDS | 3423 | X | Hunter syndrome |
| IGF1R | 3480 | 15 | Familial short statute |
| IKBKG | 8517 | X | inncontinentia pigmenti, |
| | | | hypohidrotic ectodermal dysplasia, |
| | | | and immunodeficiencies |
| IL1RAPL1 | 11141 | X | intellectual disability |
| KANSL1 | 284058 | 17 | intellectual disability |
| KAT6B | 23522 | 10 | Say-Barber-Biesecker/ |
| | | | Young-Simpson syndrome |
| KCNH2 | 3757 | 7 | long QT syndrome type 2 |
| KDM5C | 8242 | X | cognitive disability |
| KDM6A | 7403 | X | Kabuki syndrome |
| KIAA2022 | 340533 | X | cognitive disability and epilepsy |
| KIF11 | 3832 | 10 | microcephaly |
| KMT2A | 4297 | 11 | Acute lymphoid leukemias and |
| | | | acute myeloid leukemias |
| KMT2D | 8085 | 12 | Kabuki syndrome |
| L1CAM | 3897 | X | Masa syndrome and L1 syndrome |
| LAMP2 | 3920 | X | Danon disease |
| LDLR | 3949 | 19 | Familial hypercholesterolemia |
| LEMD3 | 23592 | 12 | Buschke-Ollendorff syndrome |
| | | | and melorheostosis |
| LHX4 | 89884 | 1 | hypopituitarism |
| LMNA | 4000 | 1 | cardiomyopathy |
| LRP5 | 4041 | 11 | familial exudative vitreoretinopathy |
| MAGEL2 | 54551 | 15 | Prader-Willi syndrome (PWS) |
| MAGT1 | 84061 | X | intellectual disability |
| MAOA | 4128 | X | Mental retardation |
| MAP2K2 | 5605 | 19 | cardiofaciocutaneous syndrome |
| MBD5 | 55777 | | Microcephaly, intellectual |
| | | | disabilities, speech |
| | | | impairment, and seizures |
| MECP2 | 4204 | X | Rett syndrome |
| MED13L | 23389 | 12 | Intellectual disability |
| MEF2C | 4208 | 5 | cognitive disability, epilepsy, |
| | | | and cerebral malformation |
| MEIS2 | 4212 | 15 | Intellectual disability |
| MEN1 | 4221 | 11 | Multiple Endocrine Neoplasia |
| | | | type 1 |
| MID1 | 4281 | X | Opitz syndrome |
| MLH1 | 4292 | 3 | colon cancer |
| MNX1 | 3110 | 7 | Currarino syndrome |
| MSH2 | 4436 | 2 | hereditary nonpolyposis colon |
| | | | cancer |
| MSH6 | 2956 | 2 | hereditary nonpolyposis colon |
| | | | cancer, colorectal cancer, and |
| | | | endometrial cancer |
| MTAP | 4507 | 9 | diaphyseal medullary stenosis |
| | | | with malignant fibrous |
| | | | histiocytoma (DMSMFH). |
| MTM1 | 4534 | X | X-linked myotubular myopathy |
| MYBPC3 | 4607 | 11 | familial hypertrophic |
| | | | cardiomyopathy |
| MYLK | 4638 | 3 | Megacystis Microcolon Intestinal |
| | | | Hypoperistalsis Syndrome |
| MYT1L | 23040 | 2 | schizophrenia |
| NDP | 4693 | X | Norrie disease |
| NF2 | 4771 | 22 | neurofibromatosis type II |
| NFIX | 4784 | 19 | Marshall-Smith syndrome or |
| | | | Sotos-like syndrome |
| NHS | 4810 | X | Nance-Horan syndrome |
| NIPBL | 25836 | 5 | Cornelia de Lange syndrome |
| NODAL | 4838 | 10 | Cardiovascular malformations |
| NOG | 9241 | 17 | symphalangism (SYM1) and |
| | | | multiple synostoses syndrome |
| | | | (SYNS1) |
| NR0B1 | 190 | X | congenital adrenal hypoplasia and |
| | | | hypogonadotropic hypogonadism |
| NRXN1 | 9378 | 2 | Pitt-Hopkins-like syndrome-2 |
| | | | and schizophrenia |
| NSDHL | 50814 | X | CHILD syndrome |
| NXF5 | 55998 | X | Mental retardation |
| NYX | 60506 | X | X-linked congenital stationary |
| | | | night blindness |
| OCRL | 4952 | X | oculocerebrorenal syndrome of |
| | | | Lowe and also Dent disease |
| OFD1 | 8481 | X | oral-facial-digital syndrome type |
| | | | I and Simpson-Golabi-Behmel |
| | | | syndrome type 2 |
| OPHN1 | 4983 | X | X-linked cognitive disability |
| OTC | 5009 | X | Hyperammonemia |
| OTX2 | 5015 | 14 | syndromic microphthalmia 5 and |
| | | | pituitary hormone deficiency 6 |
| PAFAH1B1 | 5048 | 17 | Lissencephaly |
| PAK2 | 5062 | 3 | intellectual disability |
| PAK3 | 5063 | X | intellectual disability |
| PCDH19 | 57526 | X | epileptic encephalopathy and |
| | | | autism |
| PDHA1 | 5160 | X | X-linked Leigh syndrome |
| PGK1 | 5230 | X | neurological impairment |
| PHEX | 5251 | X | Hypophospatemic rickets |
| PHF6 | 84295 | X | cognitive disability and epilepsy |
| PHF8 | 23133 | X | Mental retardation and cleft palate |
| PIGA | 5277 | X | encephalopathies |
| PITX3 | 5309 | 10 | Ocular and neurological disorders |
| PKP2 | 5318 | 12 | cardiomyopathy |
| PLP1 | 5354 | X | Pelizaeus-Merzbacher disease |
| | | | and spastic paraplegia type 2 |
| POLR1D | 51082 | 13 | Treacher Collins syndrome (TCS) |
| PORCN | 64840 | X | focal dermal hypoplasia |
| PQBP1 | 10084 | X | cognitive disability |
| PRPS1 | 5631 | X | Charcot-Marie-Tooth disease |
| | | | and Arts syndrome |
| PRRT2 | 112476 | 16 | paroxysmal kinesigenic |
| | | | dyskinesias |
| PTHLH | 5744 | 12 | osteochondoplsia |
| PTPN11 | 5781 | 12 | Noonan syndrome |
| RAB39B | 116442 | X | cognitive disability, epilepsy, |
| | | | and macrocephaly |
| RASA1 | 5921 | 5 | capillary malformations and |
| | | | Parkes Weber syndrome |
| RBFOX1 | 54715 | 16 | Epilepsy |
| RET | 5979 | 10 | Hirschsprung disease |
| RP2 | 6102 | X | Retinal dystrophies |
| RPS17 | 6218 | 15 | Diamond-Blackfan anemia |
| RPS24 | 6229 | 10 | Diamond-Blackfan anemia |
| RPS26 | 6231 | 12 | Diamond-Blackfan anemia |
| RPS6KA3 | 6197 | X | Coffin-Lowry syndrome |
| RS1 | 6247 | X | retinoschisis |
| SCN2A | 6326 | 2 | Epilepsy and autism |
| SCN5A | 6331 | 3 | Long QT syndrome type 3 |
| SDHAF2 | 54949 | 11 | paraganglioma |
| SDHB | 6390 | 1 | paraganglioma |
| SDHC | 6391 | 1 | paraganglioma |
| SDHD | 6392 | 11 | paraganglioma |
| SETBP1 | 26040 | 18 | Schinzel-Giedion syndrome |
| SETD5 | 55209 | 3 | Intellectual disability |
| SGCE | 8910 | 7 | Myoclonus dystonia |
| SH2B1 | 25970 | 16 | Maladaptive behaviors and obesity |
| SH2D1A | 4068 | X | Lymphoproliferative syndrome |
| SIX3 | 6496 | 2 | holoprosencephaly |
| SLC16A12 | 387700 | 10 | Juvenile cataracts and renal |
| | | | glucosuria |
| SLC16A2 | 6567 | X | Allan-Herndon-Dudley syndrome |
| SLC2A1 | 6513 | 1 | Paroxysmal exertion-induced |
| | | | dyskinesia |
| SLC4A10 | 57282 | 2 | Epilepsy and mental retardation |
| SLC6A8 | 6535 | X | Mental retardation |
| SLC9A6 | 10479 | X | cognitive disability |
| SMAD3 | 4088 | 15 | Cardiovascular malformations |
| | | | and aneurysms |
| SMAD4 | 4089 | 18 | pancreatic cancer, juvenile |
| | | | polyposis syndrome, and |
| | | | hereditary hemorrhagic |
| | | | telangiectasia syndrome |
| SMARCA4 | 6597 | 19 | Rhabdoid tumor predisposition |
| | | | syndrome |
| SMARCB1 | 6598 | 22 | Rhabdoid tumor predisposition |
| | | | syndrome |
| SMS | 6611 | X | intellectual disability |
| SNURF | 8926 | 15 | Prader-Willi Syndrome |
| SOX11 | 6664 | 2 | Autism and mental retardation |
| SOX5 | 6660 | 12 | Mental retardation |
| SPINK1 | 6690 | 5 | hereditary pancreatitis and |
| | | | tropical calcific pancreatitis |
| SRY | 6736 | Y | gonadal dysgenesis |
| STK11 | 6794 | 19 | Peutz-Jeghers syndrome and |
| | | | cancer |
| STS | 412 | X | X-linked ichthyosis (XLI) |
| STXBP1 | 6812 | 9 | infantile epileptic |
| | | | encephalopathy-4 |
| SYN1 | 6853 | | neuronal degeneration such as |
| | | | Rett syndrome |
| SYNGAP1 | 8831 | 6 | intellectual disability and autism |
| TAB2 | 23118 | 6 | congenital heart defects |
| TBX20 | 57057 | 7 | cardiac pathologies |
| TBX22 | 50945 | X | Cleft palate |
| TBX4 | 9496 | 17 | Small patella syndrome |
| TCF12 | 6938 | 15 | Anaplastic oligodendroglioma |
| TDGF1 | 6997 | 3 | forebrain defects |
| TFAP2B | 7021 | 6 | Char syndrome |
| TGFBR1 | 7046 | 9 | Ferguson-Smith disease (FSD) |
| TGFBR2 | 7048 | 3 | Syndrome, Loeys-Deitz Aortic |
| | | | Aneurysm Syndrome |
| TGIF1 | 7050 | 18 | holoprosencephaly type 4 |
| TIMM8A | 1678 | X | Jensen syndrome |
| TNNI3 | 7137 | 19 | cardiomyopathy |
| TP63 | 8626 | 3 | ectodermal dysplasia, cleft |
| | | | lip/palate, and split-hand/foot |
| | | | malformation |
| TSPAN7 | 7102 | X | cognitive disability and |
| | | | neuropsychiatric diseases |
| UBE2A | 7319 | X | cognitive disability |
| UBE3A | 7337 | 15 | autism |
| UPF3B | 65109 | X | Mental retardation |
| VEGFA | 7422 | 6 | Cardiovascular defects |
| WDR45 | 11152 | X | neurodegeneration |
| XIAP | 331 | X | dysgammaglobulinemia |
| YAP1 | 10413 | 11 | hearing loss, intellectual disability, |
| | | | hematuria, and orofacial clefting |
| ZC4H2 | 55906 | X | cognitive disability |
| ZDHHC9 | 51114 | X | cognitive disability |
| ZEB2 | 9839 | 2 | Mowat-Wilson syndrome |
| ZFPM2 | 23414 | | Cardiovascular malformations |
| ZIC1 | 7545 | 3 | Hepatocellular carcinoma |
| ZIC3 | 7547 | X | X-linked visceral heterotaxy |
| ZIC4 | 84107 | 3 | Danny-Walker malformation |
| ZNF41 | 7592 | X | cognitive disability |
| ZNF674 | 641339 | X | cognitive disability |
| ZNF711 | 7552 | X | cognitive disability |
| CACNA1A | 773 | 19 | Neurological disorders |

### VI. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Cell Culture. All experiments were performed within 10 passages of cell stock thaws. HEK293T (ATCC, CRL-11268), HeLa (ATCC, CCL-2), A549 (ATCC, CCL-185), SK-BR-3 (ATCC, HTB-30), U2OS (ATCC, HTB-96), HCT116 (ATCC, CRL-247), K562 (ATCC, CRL-243), CHO-K1 (ATCC, CCL-61), ARPE-19 (ATCC, CRL-2302), HFF (ATCC, CRL-2429), Jurkat-T (ATCC, TIB-152), hTERT-MSC (ATCC, SCRC-4000), and Neuro-2a (ATCC, CCL-131) cells were purchased from American Type Cell Culture (ATCC, USA) and cultured in ATCC-recommended media supplemented with 10% FBS (Sigma-Aldrich) and 1% pen/strep (100 units/mL penicillin, 100 μg/mL streptomycin; Gibco) at 37° C. and 5% CO₂. NIH3T3 cells were a kind gift from Dr. Caleb Bashor's lab and were cultured in DMEM supplemented with 10% FBS (Sigma-Aldrich) and 1% pen/strep (100 units/mL penicillin, 100 μg/mL streptomycin) at 37° C. and 5% CO₂.

Plasmid Transfection and Nucleofection. HEK293T cell transfections were performed in 24-well plates using 375 ng of dCas9 expression plasmid and 125 ng of equimolar pooled or individual gRNAs/crRNAs. 1.25×10⁵ HEK293T cells were plated the day before transfection and then transfected using Lipofectamine 3000 (Invitrogen, USA) as per manufacturer's instruction. For two component systems (dCas9+MCP or dCas9+scFv systems) 187.5 ng of each plasmid was used. For multiplex gene activation experiments using DREAM platforms, 25 ng of each gRNA encoding plasmid targeting each respective gene was used. Transfections in HeLa, A549, SK-BR-3, U2OS, HCT-116, HFF, NIH3T3, and CHO-K1 were performed in 12-well plates using Lipofectamine 3000 and 375 ng dCas9 plasmid, 375 ng of MCP-effector fusion proteins, and 250 ng DNA of MS2-modified gRNA encoding plasmid. For transfections using dCas12a fusion proteins where single genes were targeted, 375 ng of dCas12a-effector fusion plasmids and 125 ng of crRNA plasmids were transfected using lipofectamine 3000 per manufacturer's instruction. For multiplex gene activation experiments using dCas12a, 375 ng of dCas12a-effector fusion encoding plasmid and 250 ng of multiplex crRNA expression plasmids were used. For experiments using E. coli and P. aeruginosa Type I CRISPR systems, the inventors followed the same stoichiometries used in previous studies. For transfection of ICAM1-ZF effectors, 500 ng of each ICAM1 targeting ZF fusion was transfected. Transfections using IL1RN-TALE fusion proteins were performed using 500 ng of either single TALE or a pool of 4 TALEs using 125 ng of each TALE fusion. All ZF and TALE transfections were performed in HEK293T cells in 24-well format using Lipofectamine 3000 as per manufacturers instruction. For K562 cells, 1×10⁶ cells were nucleofected using the Lonza SF Cell Line 4D-Nucleofector Kit (Lonza V4XC-2012) and a Lonza 4D Nucleofector (Lonza, AAF1002X) using the FF-120 program. 2000 ng of total plasmids were nucleofected in each condition using 1×10⁶ K562 cells and 667 ng each of; dCas9 plasmid, MCP fusion plasmid, and pooled MS2-sgRNA expression plasmid was nucleofected per condition. Immediately after nucleofection, K562 cells were transferred to prewarmed media containing 6-well plates. hTERT-MSCs were electroporated with using the Neon transfection system (Thermo Fisher Scientific) using the 100 μL kit. 5×10⁵ hTERT-MSCs were resuspended in 100 μL resuspension buffer R and 10 μg total DNA (3.75 μg dCas9, 3.75 μg MCP-fusion effector plasmid, and 2.5 μg MS2-modified gRNA encoding plasmid). Electroporation was performed using the settings recommended by the manufacturers for mesenchymal stem cells: Voltage: 990V, Pulse width: 40 ms, Pulse number: 1. For fibroblast reprogramming experiments, the inventors used the Neon transfection system using the amounts of endotoxin free DNA described previously¹⁷ and below. Dual AAV (500 ng of each) and All-in-one (AIO) AAV (1 μg) construct transfections were performed in Neuro-2a cells in 12-well format using Lipofectamine 3000 as per manufacturers instruction.

PBMC Isolation, Culture, and Nucleofection. De-identified white blood cell concentrates (buffy coats) were obtained from the Gulf Coast Regional Blood Center in Houston, Texas. PBMCs were isolated from buffy coats using Ficoll gradient separation and cryopreserved in liquid nitrogen until later use. 1×10⁶ PBMCs per well were stimulated for 48h in a CD3/CD28 (Tonbo Biosciences, 700037U100 and 70289U100, respectively)-coated 24-well plate containing RPMI media supplemented with 10% FBS (Sigma-Aldrich), 1% Pen/Strep (Gibco), 10 ng/ml IL-15 (Tonbo Biosciences, 218157U002), and 10 ng/mL IL-7 (Tonbo Biosciences, 218079U002). Stimulated PBMCs were electroporated using the Neon transfection system (Thermo Fisher Scientific) 100 μL kit per manufacturer protocol. Briefly, PBMCs were centrifuged at 300 g for 5 min and resuspended in Neon Resuspension Buffer T to a final density of 1×10⁷ cells/mL. 100 μL of the resuspended cells (1×10⁶ cells) were then mixed with 12 μg total plasmid DNA (4.5 μg of dCas9 fusion encoding plasmids, 4.5 μg of MCP fusion encoding plasmids, and 3 μg of four equimolar pooled MS2-modified gRNA encoding plasmids) and electroporated with the following program specifications using a 100 μL Neon Tip: pulse voltage 2,150v, pulse width 20 ms, pulse number 1. Endotoxin free plasmids were used in all experiments. After electroporation, PBMCs were incubated in prewarmed 6-well plates containing RPMI media supplemented with 10% FBS (Sigma-Aldrich), 1% Pen/Strep (Gibco), 10 ng/mL IL-15, and 10 ng/mL IL-7. PBMCs were maintained at 37° C., 5% CO₂ for 48h before RNA isolation and QPCR.

Human Primary T Cell and Primary Umbilical Cord MSC Culture and Lentiviral Transduction. PBMCs were isolated from de-identified white blood cell concentrates (buffy coats) using Ficoll gradient separation. T cells were isolated using negative selection via the EasySep™ Human T Cell Isolation Kit (StemCell, 17951). T cells were frozen in Bambanker Cell Freezing Media (Bulldog Bio Inc, BB01) and stored in liquid nitrogen until use. Umbilical cord derived MSCs (ATCC, PCS-500-010) were cultured in MSC basal media (ATCC, PCS-500-030) supplemented with Mesenchymal Stem Cell Growth Kit (PCS-500-040) containing rhFGF basic (5 ng/ml), rhFGF acidic (5 ng/mL), rhEGF (5 ng/mL), FBS (2%), and L-Alanyl-L-Glutamine (2.4 mM). MSC media was also supplemented with 1% Pen-strep (Gibco, 15140122). MSCs were maintained at 37° C., 5% CO₂. Lentiviral transduction was performed in stimulated T cells as previously described³⁴. Briefly, 1×10⁶ T cells per well were stimulated for 24 h with Dynabeads™ Human T-Activator CD3/CD28 for T Cell Expansion and Activation (Thermo Fisher Scientific, 11161D) according to manufacturer's instructions in a 24-well plate containing X-VIVO 15 media (Lonza, 04418Q) supplemented with 5% FBS (Sigma-Aldrich), 55 mM 2-Mercaptoethanol (Gibco, 21985023), 4 mM N-acetyl-L-cysteine (Thermo Fisher Scientific, 160280250), and 500 IU/ml of recombinant human IL-2 (Biolegend, 589104). Stimulated T cells were co-transduced via spinoculation at 931×g, 37° C. for 2 hours in a plate coated with Retronectin (Takara Bio, T100B) with an MOI of ˜5.0 for each lentivirus (dCas9 lentivirus at MOI ˜5.0 and gRNA-MCP-fusion effector lentivirus). After spinoculation, T cells were maintained at 37° C., 5% CO₂ for 48h before downstream experiments. MSCs were co-transduced with an MOI of ˜10.0 (dCas9 lentivirus at MOI ˜10.0 and gRNA-MCP-fusion effector lentivirus at MOI ˜10.0) for each lentivirus via reverse transduction by seeding 1.25×10⁵ cells into each well of a 12-well plate containing the virus in MSC media supplemented with 8 μg/mL polybrene. Media was changed after 16 hours. Further experimental analyses were performed 72 hours post-transduction.

Mouse Primary Neuron Culture and AAV8 transduction. Mouse C57 Cortex Neurons (Lonza, M-CX-300) were cultured in Primary Neuron Basal Medium (PNBM) supplemented with 2 mM L-glutamine, GA-1000 and 2% NSF. In brief, 4×10⁵ cells were seeded in poly-D-lysine and laminin coated 24 well plates and cultured for 7 days for neuronal differentiation. On day 8, cells from each well were transduced with 1×10¹⁰ AAV8 viral particles (2.5×10⁴/cell). 5 days post-transduction cells were harvested for RNA isolation and QPCR analysis.

Plasmid Cloning. Lenti-dCas9-VP64 (Addgene #61425), dCas9-VPR (Addgene #63798), dCas9-p300 (Addgene #83889), MCP-p65-HSF1 (Addgene #61423), scFv-VP64 (Addgene #60904), SpgRNA expression plasmid (Addgene #47108), MS2-modified gRNA expression plasmid (Addgene #61424), AsCas12a (Addgene #128136), E. Coli Type I Cascade system (Addgene #106270-106275) and Pae Type I Cascade System (Addgene #153942 and 153943), YAP-S5A (Addgene #33093) have been described previously. The eNRF2 TAD fusion was synthetically designed and ordered as a gBlock from IDT. To generate an isogenic C-terminal effector domain cloning backbone, the dCas9-p300 plasmid (Addgene #83889) was digested with BamHI and then a synthetic double-stranded ultramer (IDT) was incorporated using NEBuilder HiFi DNA Assembly (NEB, E2621) to generate a dCas9-NLS-linker-BamHI-NLS-FLAG expressing plasmid. This plasmid was further digested with AfeI and then a synthetic double-stranded ultramer (IDT) was incorporated using NEBuilder HiFi DNA Assembly to generate a FLAG-NLS-MCS-linker-dCas9 expressing Plasmid for N-terminal effector domain cloning. For fusion of effector domains to MCP, the MCP-p65-HSF1 plasmid (Addgene #61423) was digested with BamHI and NheI and respective effector domains were cloned using NEBuilder HiFi DNA Assembly. For SunTag components, the scFv-GCN4-linker-VP16-GB1-Rex NLS sequence was PCR amplified from pHRdSV40-scFv-GCN4-sfGFP-VP64-GB1-NLS (Addgene #60904) and cloned into a lentiviral backbone containing an EF1-alpha promoter. Then VP64 domain was removed and an AfeI restriction site was generated and used for cloning TADs using NEBuilder HiFi DNA Assembly. The pHRdSV40-dCas9-10×GCN4_v4-P2A-BFP (Addgene #60903) vector was used for dCas9-based scFv fusion protein recruitment to target loci. All MTF TADs were isolated using PCR amplified from a pooled cDNA library from HEK293T, HeLa, U2OS and Jurkat-T cells. TADs were cloned into the MCP, dCas9 C-terminus, dCas9 N-terminus, and scFv backbones described above using NEBuilder HiFi DNA Assembly. Bipartite N-terminal fusions between MCP-MRTF-A or MCP-MRTF-B TADs and STAT 1-6 TADs were generated by digesting the appropriate MCP-fusion plasmid (MCP-MRTF-A or MCP-MRTF-B) with BamHI and then subcloning PCR-amplified STAT 1-6 TADs using NEBuilder HiFi DNA Assembly. Bipartite C-terminal fusions between MCP-MRTF-A or MCP-MRTF-B TADs and STAT 1-6 TADs were generated by digesting the appropriate MCP-fusion plasmid (MCP-MRTF-A or MCP-MRTF-B) with NheI and then subcloning PCR-amplified STAT 1-6 TADs using NEBuilder HiFi DNA Assembly. Similarly, eNRF2 was fused to the N- or C-terminus of the bipartite MRTF-A-STAT1 TAD in the MCP-fusion backbone using either BamHI (N-terminal; MCP-eNRF2-MRTF-A-STAT1 TAD) or NheI (C-terminal; MCP-MRTF-A-STAT1-eNRF2 TAD) digestion and NEBuilder HiFi DNA Assembly to generate the MCP-NMS or MCP-MSN tripartite TAD fusions, respectively. SadCas9 (with D10A and N580A mutations derived using PCR) was PCR amplified and then cloned into the SpdCas9 expression plasmid backbone created in this study digested with BamHI and XbaI. This SadCas9 expression plasmid was digested with BamHI and then PCR-amplified VP64 or VPR TADs were cloned in using NEBuilder HiFi DNA Assembly. CjCas9 was PCR-amplified from pAAV-EFS-CjCas9-eGFP-HIF1a (Addgene #137929) as two overlapping fragments using primers to create D8A and H559A mutations. These two CjdCas9 PCR fragments were then cloned into the SpdCas9 expression plasmid digested with BamHI and XbaI using NEBuilder HiFi DNA Assembly. This CjdCas9 expression plasmid was digested with BamHI and the PCR-amplified VP64 or VPR TADs were cloned in using NEBuilder HiFi DNA Assembly. HNH domain deleted SpdCas9 plasmids were generated using different primer sets designed to amplify the N-terminal and C-terminal portions of dCas9 excluding the HNH domain and resulting in either: no linker, a glycine-serine linker, or an XTEN16 linker, between HNH-deleted SpdCas9 fragments. These different PCR-amplified regions were cloned into the SpdCas9 expression plasmid digested with BamHI and XbaI using NEBuilder HiFi DNA Assembly. MCP-mCherry, MCP-MSN and MCP-p65-HSF1 were digested with NheI and a single strand oligonucleotide encoding the FLAG sequence was cloned onto the C-terminus of each respective fusion protein using NEBuilder HiFi DNA Assembly to enable facile detection via Western blotting. 1× 9aa TADs were designed and annealed as double strand oligos and then cloned into the BamHI/NheI-digested MCP-p65-HSF1 backbone plasmid (Addgene #61423) using T4 ligase (NEB). Heterotypic 2× 9aa TADs were generated by digesting MCP-1× 9aa TAD plasmids with either BamHI or NheI and then cloning single strand DNA encoding 1× 9aa TADs to the N- or C-termini using NEBuilder HiFi DNA Assembly. Heterotypic MCP-3× 9aa TADs were generated similarly by digesting MCP-2× 9aa TAD containing plasmids either with BamHI or NheI and then single strand DNA encoding 1× 9aa TADs were cloned to the N- or C-termini using NEBuilder HiFi DNA Assembly. Selected fusions between 3× 9aa TADs and eNRF2 were generated using gBlock (IDT) fragments and cloned into the BamHI/NheI-digested MCP-p65-HSF1 backbone plasmid (Addgene #61423) using NEBuilder HiFi DNA Assembly. To generate mini-DREAM compact single plasmid system, SpdCas9-HNH (no linker) deleted plasmid was digested with BamHI and then PCR amplified P2A self-cleaving sequence and MCP-eNRF2-3× 9aa TAD (eN3×9) was cloned using NEBuilder HiFi DNA Assembly. For dCas12a fusion proteins, SiT-Cas12a-Activ (Addgene #128136) was used. First, the inventors generated a nuclease dead (E993A) SiT-Cas12a backbone using PCR amplification and the inventors used this plasmid for subsequent C-terminal effector cloning using BamHI digestion and NEBuilder HiFi DNA Assembly. For E. coli Type I CRISPR systems, the Cas6-p300 plasmid (Addgene #106275) was digested with BamHI and then MSN and NMS domains were cloned in using NEBuilder HiFi DNA Assembly. Pae Type I Cascade plasmids encoding Csy1-Csy2 (Addgene #153942) and Csy3-VPR-Csy4 (Addgene #153943) were obtained from Addgene. The Csy3-VPR-Csy4 plasmid was digested with MluI (NEB) and BamHI (to remove the VPR TAD) and then the nucleoplasmin NLS followed by a linker sequence was added using NEBuilder HiFi DNA Assembly. Next, this Csy3-Csy4 plasmid was digested with AscI and either the MSN or NMS TADs were cloned onto the N-terminus of Csy3 NEBuilder HiFi DNA Assembly. ZF fusion proteins were generated by cloning PCR-amplified MSN, NMS, or VPR domains into the BsiWI and AscI digested ICAM1 targeting ZF-p300 plasmid¹⁰ using NEBuilder HiFi DNA Assembly. Similarly, TALE fusion proteins were created by cloning PCR-amplified MSN, NMS, or VPR domains into the BsiwI and AscI digested IL1RN targeting TALE plasmid backbone¹⁰ using NEBuilder HiFi DNA Assembly. pCXLE-dCas9VP192-T2A-EGFP-shP53 (Addgene #69535), GG-EBNA-OSK2M2L1-PP (Addgene #102898) and GG-EBNA-EEA-5guides-PGK-Puro (Addgene #102898) used for reprogramming experiments have been described previously^{17, 35}. The PCR-amplified NMS domain was cloned into the sequentially digested (XhoI then SgrDI; to remove the VP192 domain) pCXLE-dCas9VP192-T2A-EGFP-shP53 backbone using NEBuilder HiFi DNA Assembly. TADs were directly fused to the C-terminus of dCas9 by digesting the dCas9-NLS-linker-BamHI-NLS-FLAG plasmid with BamHI and then cloning in PCR-amplified TADs using NEBuilder HiFi DNA Assembly. TADs were directly fused to the N-terminus to dCas9 by digesting the FLAG-NLS-MCS-linker-dCas9 plasmid with AgeI (NEB) and then cloning in PCR-amplified TADs using NEBuilder HiFi DNA Assembly. For constructs harboring both N- and C-terminal fusions, respective plasmids with TADs fused to the C-terminus of dCas9 were digested with AgeI and then PCR-amplified TADs were cloned onto the N-terminus of dCas9 using NEBuilder HiFi DNA Assembly. hSyn-AAV-EGFP (Addgene #50465) plasmid was used to generate different AAV based DNA constructs. For SpdCas9 cloning both EFGP and WPRE were removed using XbaI and XhoI and SpdCas9 and the modified smaller WPRE along with SV40 polyA signal (W3SL) were then cloned into this backbone using NEBuilder HiFi DNA Assembly. For expression of MS2-gRNA and hSyn-MCP-MSN from a single plasmid, both components were PCR amplified and cloned into an EGFP-removed hSyn-AAV-EGFP backbone using NEBuilder HiFi DNA Assembly. For All-In-One AAV backbone the M11 promoter³⁶ was used to drive SaCas9 gRNA expression. The SCP1³⁷ and the EFS promoters were used to drive the expression of NMS-SadCas9. The efficient, smaller synthetic WPRE and polyadenylation signal CW3SA³⁸ was utilized to maximize expression this size-limited context. Following cloning and sequence verification, 3 SaCas9 specific gRNAs targeting mouse Agrp gene were cloned into the all-in-one (AIO) vectors using Bbs1 restriction digestion. Following identification of the most efficacious gRNA (by transfecting into Neuro-2a cells), the SCP1 and EFS promoter driven SadCas9 based AIO plasmids were sequence verified by Plasmidsaurus. Sequence verified SpdCas9 and SCP1 and EFS promoter driven SadCas9 based AIO plasmids were sent to Charles River Laboratories for AAV8 production. Titers of different AAVs are included in source data.

gRNA Design and Construction. All protospacer sequences for SpCas9 systems were designed using the Custom Alt-R® CRISPR-Cas9 guide RNA design tool (IDT). All gRNA protospacers were then phosphorylated, annealed, and cloned into chimeric U6 promoter containing sgRNA cloning plasmid (Addgene #47108) and/or an MS2 loop containing plasmid backbone (Addgene #61424) digested with Bbs1 and treated with alkaline phosphatase (Thermo) using T4 DNA ligase (NEB). The SaCas9 gRNA expression plasmid (pIBH072) was a kind gift from Charles Gersbach and was digested with BbsI or Bpil (NEB or Thermo, respectively) and treated with alkaline phosphatase and then annealed protospacer sequences were cloned in using T4 DNA ligase (NEB). gRNAs were cloned into the pU6-Cj-sgRNA expression plasmid (Addgene #89753) by digesting the vector backbone with BsmBI or Esp3I (NEB or Thermo, respectively), and then treating the digested plasmid with alkaline phosphatase, annealing phosphorylated gRNAs, and then cloning annealed gRNAs into the backbone using T4 DNA ligase. MS2-stem loop containing plasmids for SaCas9 and CjCas9 were designed as gBlocks (IDT) with an MS2-stem loop incorporated into the tetraloop region for both respective gRNA tracr sequences. crRNA expression plasmids for the Type I Eco Cascade system were generated by annealing synthetic DNA ultramers (IDT) containing direct repeats (DRs) and cloning these ultramers into the BbsI and SacI-digested SpCas9 sgRNA cloning plasmid (Addgene #47108) using NEBuilder HiFi DNA Assembly. crRNA expression plasmids for Pae Type I Cascade system were generated by annealing and then PCR-extending overlapping oligos (that also harbored a BsmBI or Esp3I cut site for facile crRNA array incorporation) into the sequentially BbsI (or Bpil) and SacI-digested SpCas9 sgRNA cloning plasmid (Addgene #47108) using NEBuilder HiFi DNA Assembly. crRNA expression plasmids for Cas12a systems were generated by annealing and then PCR-extending overlapping oligos (that also harbored a BsmBI or Esp3I cut site for facile crRNA array incorporation) into the sequentially BbsI (or Bpil) and SacI-digested SpCas9 sgRNA cloning plasmid (Addgene #47108) using NEBuilder HiFi DNA Assembly.

crRNA Array Cloning. crRNA arrays for AsCas12a and Type I CRISPR systems were designed in fragments as overlapping ssDNA oligos (IDT) and 2-4 oligo pairs were annealed. Oligos were designed with an Esp3I cut site at 3′ of the array for subsequent cloning steps. Equimolar amounts of oligos were mixed, phosphorylated, and annealed similar to the standardized gRNA/crRNA assembly protocol above. Phosphorylated and annealed arrays were then cloned into the respective Esp3I-digested and alkaline phosphatase treated crRNA cloning backbone (described above) using T4 DNA ligase (NEB). crRNA arrays were verified by Sanger sequencing. Correctly assembled 4-8 crRNA array expressing plasmids were then digested again with Esp3I and alkaline phosphatase treated to enable incorporation of subsequent arrays up to 20 crRNAs.

Lentiviral packaging. All lentiviral transfer and packaging plasmids were purified using the Endofree Plasmid Maxi Kit (Qiagen, 12362). Lentivirus was packaged as previously described³⁴ with minor modifications. Briefly, HEK293T cells were seeded into 225 mm flasks and maintained in DMEM. OptiMem was used for transfection and Sodium butyrate was added to a final concentration of 4 mM. Lentivirus was then concentrated 100× using the Lenti-X concentrator (Takara Bio, 631232). Biological titration of lentivirus by QPCR was carried out as previously described³⁹, with the following modifications. Volumes of 10, 5, 1, 0.1, 0.01, and 0 μl of concentrated lentiviral particles were reverse transduced into 5×10⁴ HEK293T cells with 8 μg/mL polybrene (Millipore-Sigma, TR1003G) in 24 well format with media exchanged after 14 hrs of transduction. gDNA was extracted 96 hours post transduction using the DNeasy Blood & Tissue Kit (Qiagen, 69506). qPCR was performed using 67.5 ng of gDNA for each condition in 10 ul reactions using Luna Universal qPCR Master Mix (NEB, M3003E).

Western Blotting. Cells were lysed in RIPA buffer (Thermo Scientific, 89900) with 1× protease inhibitor cocktail (Thermo Scientific, 78442), lysates were cleared by centrifugation and protein quantitation was performed using the BCA method (Pierce, 23225). 15-30 μg of lysate were separated using precast 7.5% or 10% SDS-PAGE (Bio-Rad) and then transferred onto PVDF membranes using the Transblot-turbo system (Bio-Rad). Membranes were blocked using 5% BSA in 1×TBST and incubated overnight with primary antibody (anti-Cas9; Diagenode #C15200216, Anti-FLAG; Sigma-Aldrich #F1804, anti-β-Tubulin; Bio-Rad #12004166). Then membranes were washed with 1×TBST 3 times (10 mins each wash) and incubated with respective HRP-tagged secondary antibodies for 1 hr. Next membranes were washed with 1×TBST 3 times (10 mins each wash). Membranes were then incubated with ECL solution (BioRad #1705061) and imaged using a Chemidoc-MP system (BioRad). The β-tubulin antibody was tagged with Rhodamine (Bio-Rad #12004166) and was imaged using Rhodamine channel in Chemidoc-MP as per manufacturer's instruction.

Quantitative Reverse-transcriptase PCR (QPCR). RNA (including pre-miRNA) was isolated using the RNeasy Plus mini kit (Qiagen #74136). 500-2000 ng of RNA (quantified using Nanodrop 3000C; Thermo Fisher) was used as a template for cDNA synthesis (Bio-Rad #1725038). cDNA was diluted 10× and 4.5 μL of diluted cDNA was used for each QPCR reaction in 10 μL reaction volume. Real-Time quantitative PCR was performed using SYBR Green mastermix (Bio-Rad #1725275) in the CFX96 Real-Time PCR system with a C1000 Thermal Cycler (Bio-Rad). Results are represented as fold change above control after normalization to GAPDH in all experiments using human cells. For murine cells, 18s IRNA was used for normalization. For CHO-K1 cells, GnbI was used for normalization. Undetectable samples were assigned a Ct value of 45 cycles.

Mature miRNA isolation and QPCR for miRNAs. Mature miRNA (miRNA) was isolated using the miRNA isolation kit (Qiagen #217084). 500 ng of isolated miRNA was polyadenylated using poly A polymerase (Quantabio #95107) in 10 μL reactions per sample and then used for cDNA synthesis using qScript Reverse Transcriptase and oligo-dT primers attached to unique adapter sequences to allow specific amplification of mature miRNA using QPCR in a total 20 μL reaction (Quantabio #95107). cDNA was diluted and 10 ng of miRNA cDNA was used for QPCR in a 25 μL reaction volume. PerfeCTa SYBR Green SuperMix (Quantabio #95053), miR-146a specific forward primer, and PerfeCTa universal reverse primer was used to perform QPCR. U6 snRNA was used for normalization.

Immunofluorescence Microscopy. Human foreskin fibroblasts (HFFs; CRL-2429, ATCC) and HFF-derived iPSCs were grown in Geltrex (Gibco, A1413302) coated 12-well plates and were fixed with 3.7% formaldehyde and then blocked with 3% BSA in 1×PBS for 1 hr at Room Temperature prior to imaging. Primary antibodies for SSEA-4 (CST #43782), TRA1-60 (CST #61220) and TRA1-81 (CST #83321) were diluted in 1% BSA in 1×PBS and incubated overnight at 4° C. The next day, cells were washed with 1×PBS, incubated with appropriate Alexaflour-488 conjugated secondary antibodies for 1 hr at Room Temperature and then washed again with 1×PBS. Cells were then incubated with DAPI (Invitrogen #D1306) containing PBS for 10m, washed with 1×PBS, and then imaged using a Nikon ECLIPSE Ti2 fluorescent microscope.

Fibroblast Reprogramming. HFFs were cultured in 1×DMEM supplemented with 1×Glutamax (Gibco, 35050061) for two passages before transfection with respective components. Cells were grown in 15 cm dishes (Corning), and detached using TrypLE select (Gibco, #12563011). Single cell suspensions were washed with complete media and then with 1×PBS. For each 1×10⁶ cells, a total of 6 μg of endotoxin free plasmids (Macherey-Nagel, 740424; 2 μg CRISPR activator plasmid, 2 μg of pluripotency factor targeting gRNA plasmid, and 2 μg of EEA-motif targeting gRNA expression plasmids) were nucleofected using a 100 μL Neon transfection tip in R buffer using the following settings: 1650V, 10 ms, and 3 pulses. Nucleofected fibroblasts were then immediately transferred to Geltrex (Gibco) coated 10 cm cell culture dishes in prewarmed media. The next day media was exchanged. 4 days later, media was replaced with iPSC induction media¹⁷. Induction media was then exchanged every other day for 18 days. After 18 days iPSC colonies were counted, and colonies picked using sterile forceps and then transferred to Geltrex coated 12-well plates. iPSC colonies were maintained in complete E8 media and passaged as necessary using ReLeSR passaging reagent (Stem Cell Technology, #05872). RNA was isolated from iPSC clones using the RNeasy Plus mini kit (Qiagen #74136) and colonies were immunostained using indicated antibodies and counterstained with DAPI (Invitrogen) for nuclear visualization.

RNA Sequencing (RNA-seq). RNA-seq was performed in duplicate for each experimental condition. 72 hrs post-transfection RNA was isolated using the RNeasy Plus mini kit (Qiagen). RNA integrity was first assessed using a Bioanalyzer 2200 (Agilent) and then RNA-seq libraries were constructed using the TruSeq Stranded Total RNA Gold (Illumina, RS-122-2303). The qualities of RNA-seq libraries were verified using the Tape Station D1000 assay (Tape Station 2200, Agilent Technologies) and the concentration of RNA-seq libraries were checked again using real time PCR (QuantStudio 6 Flex Real time PCR System, Applied Biosystem). Libraries were normalized and pooled prior to sequencing. Sequencing was performed using an Illumina Hiseq 3000 with paired end 75 base pair reads. Reads were aligned to the human genome (hg38) Gencode Release 36 reference using STAR aligner (v2.7.3a). Transcript levels were quantified to the reference genome using a Bayesian approach. Normalization was done using counts per million (CPM) method. Differential expression was done using DESeq2 (v3.5) with default parameters. Genes were considered significantly differentially expressed based upon a fold change >2 or <−2 and an FDR <0.05.

9aa TAD Prediction. 9aa TADs were predicted using previously described software (world-wide-web at at.embnet.org/toolbox/9aatad/.) 40 using the “moderately stringent pattern” criteria and all “refinement criteria” and only TADs with 100% matches were then selected for evaluation in MCP fusion proteins.

Toxicity Assays. Cellular toxicity assays in primary T cells were performed 72 hours post-transduction using the Annexin V: PE Apoptosis Detection Kit (BD Biosciences, 559763). In brief, cells were stained with 7-AAD and Annexin V: PE according to the manufacturer's protocol. Stained cell fluorescence was measured using a Sony SA3800 spectral analyzer. EGFP positive single cells were gated and assessed for 7-AAD and Annexin V: PE fluorescence. All conditions were measured in biological triplicate and measured in technical duplicate. The toxicity of treatment groups was compared to the negative control (dCas9 alone), camptothecin (5 mM), and 65° C. heat shock were used as positive controls of apoptosis and membrane permeability respectively.

Data Analysis. All data used for statistical analysis had a minimum 3 biological replicates. Data are presented as mean±SEM Gene expression analyses were conducted using Student's t-tests (Two-tailed pair or multiple unpaired). Results were considered statistically significant when the P-value was <0.05. All bar graphs, error bars, and statistics were generated using GraphPad Prism v 9.0.

### Example 2—Results

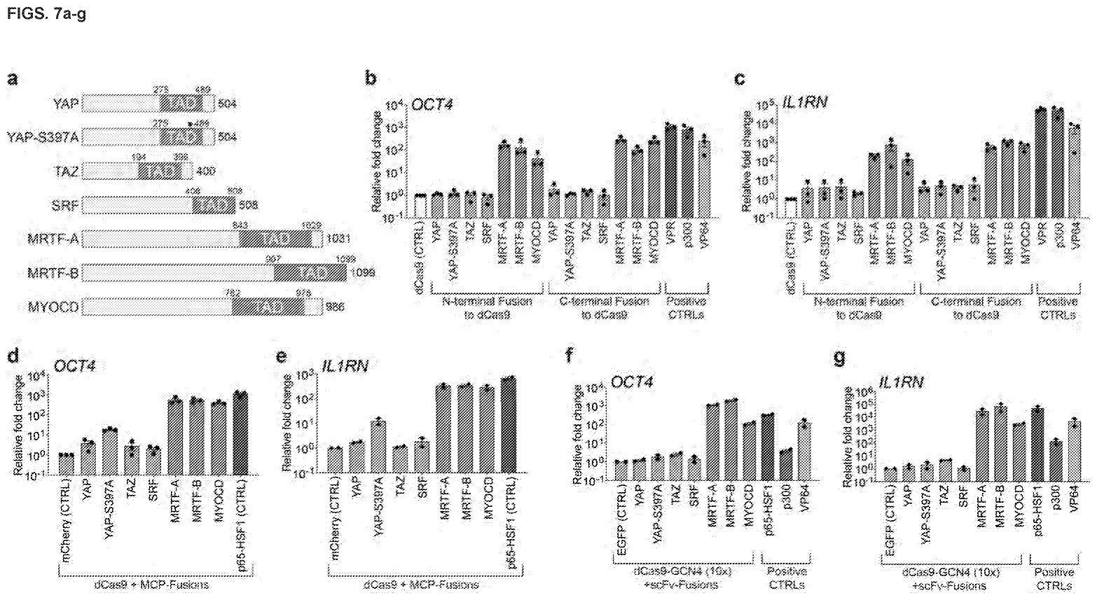

Select TADs from MTFs can activate transcription from diverse endogenous human loci when recruited by dCas9. The inventors first isolated TADs from 7 different serum-responsive MTFs (YAP, YAP-S397A41, TAZ, SRF, MRTF-A, MRTF-B, and MYOCD) and analyzed their ability to activate transcription when recruited to human promoters using either N- or C-terminal fusion to Streptococcus pyogenes dCas9 (dCas9), SunTag-mediated recruitment¹⁴, or recruitment via a gRNA aptamer and fusion to the MCP protein¹⁵ (FIGS. 7a-g). TADs derived from MRTF-A, MRTF-B, or MYOCD displayed consistent transactivation potential across recruitment architectures. The inventors next compared the optimal recruitment strategies for MRTF-A and MRTF-B TADs because they were more potent than, or comparable to, the MYOCD TAD yet slightly smaller. These results demonstrated that TADs from MRTF-A and B functioned best when fused to the MCP protein and recruited via gRNA aptamers (FIGS. 8a-f), and further that this strategy could be used with pools or single gRNAs, and to activate enhancer RNAs (eRNAs) and long noncoding RNAs (lncRNAs).

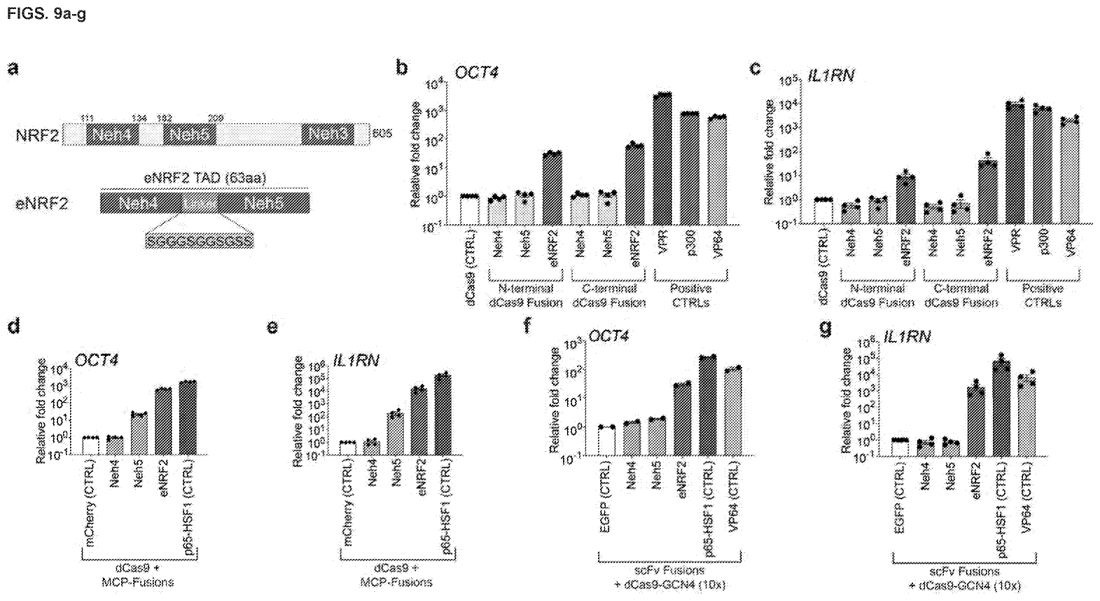

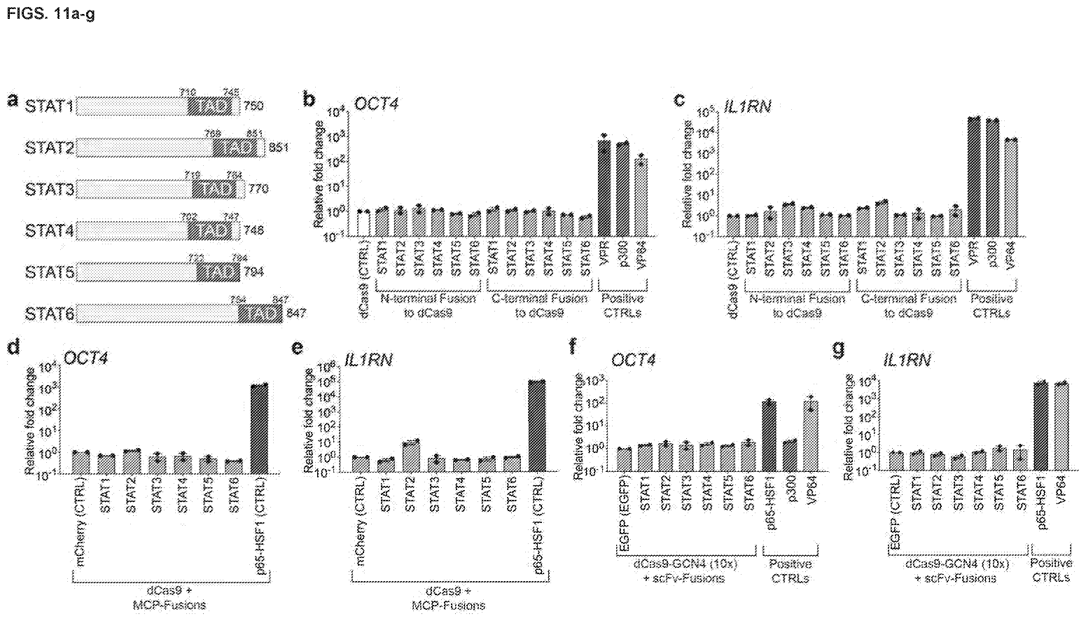

Although the NRF2-ECH homology domains 4 and 5 (Neh4 and Neh5, respectively) within the oxidative stress/antioxidant regulated NRF2 human MTF have been shown to activate gene expression in Gal4 systems²⁷, the inventors observed that neither Neh4 nor Neh5 were capable of potent human gene activation when recruited to promoters in any dCas9-based architecture (FIGS. 9a-g). Therefore, the inventors constructed an engineered TAD called eNRF2, consisting of Neh4 and Neh5 separated by an extended glycine-serine linker and found that the eNRF2 TAD stimulated high levels of transactivation in all dCas9-based recruitment configurations (FIGS. 9a-g). Similar to the MRTF-A/B TADs, eNRF2 displayed optimal potency in the gRNA aptamer/MCP-based recruitment architecture and transactivated diverse human regulatory loci (FIGS. 10a-e). The inventors next tested whether TADs derived from one of 6 different cytokine regulated/JAK-STAT family MTFs (STAT1-6) could transactivate human genes but observed that single STAT TADs alone were incapable of potent transactivation regardless of dCas9-based recruitment context (FIGS. 11a-g). Nevertheless, these data demonstrate that TADs from human MTFs can transactivate human loci when recruited via dCas9 and that these TADs are amenable to protein engineering.

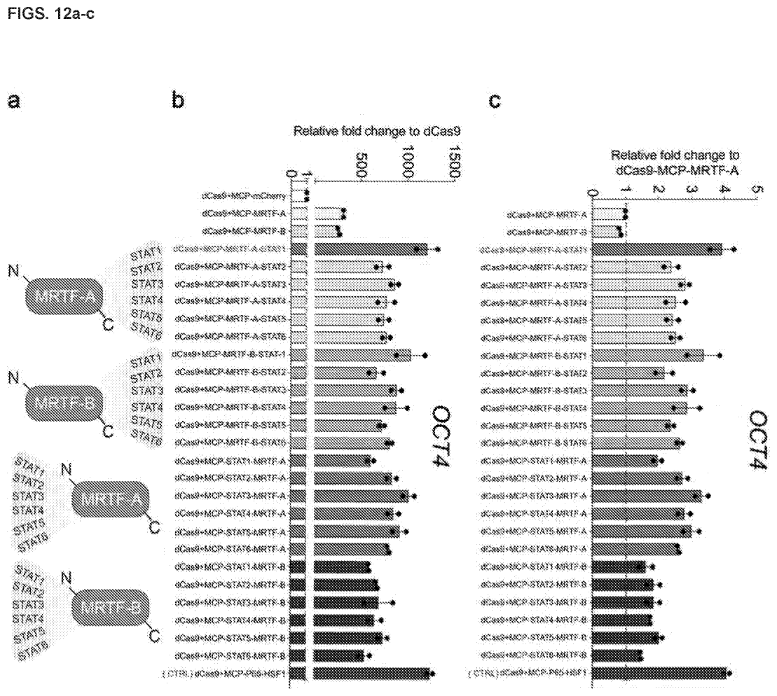

Combinations of TADs from MTFs can potently activate human genes when recruited by dCas9. STAT proteins typically activate gene expression in combination with co-factors⁴². Therefore, the inventors tested if TADs from different STAT proteins might synergize with other MTF TADs. The inventors built 24 different bipartite fusion proteins by linking each STAT TAD to the N- or C-terminus of either the MRTF-A or MRFT-B TAD and then assayed the relative transactivation potential of each bipartite fusion when recruited to the human OCT4 promoter using gRNA aptamer/MCP-based recruitment (FIGS. 12a-c). Each of these 24 fusions markedly outperformed TADs from MRTF-A/B or STAT TADs alone, and one bipartite TAD configuration (MRFT-A/STAT1) was comparable to MCP fused to the dCas9-SAM derived bipartite p65-HSF1¹⁵ module. The inventors next investigated whether the eNRF2 TAD could further enhance the potency of the MRFT-A/STAT1 module by building tripartite fusions consisting of MRTF-A/STAT1/eNRF2 (MSN) or eNRF2/MRTF-A/STAT1 (NMS) TADs. Both MSN and NMS stimulated OCT4 mRNA synthesis to levels comparable to the state-of-the-art CRISPRa platforms (FIGS. 13a-b) when recruited to the OCT4 promoter using gRNA aptamers/MCP-based targeting. Surprisingly, this potency was not further enhanced by the direct fusion of other TADs to the C-terminus of dCas9 (FIG. 13c). Collectively, these data show that gRNA aptamer/MCP-based recruitment of the MSN or NMS modules—termed the CRISPR-dCas9 recruited enhanced activation module (DREAM) platform—can efficiently stimulate transcription without viral components. These results also demonstrate that natural and engineered human TADs can have non-obvious interactions when combinatorially recruited in bi- and tripartite fashions.

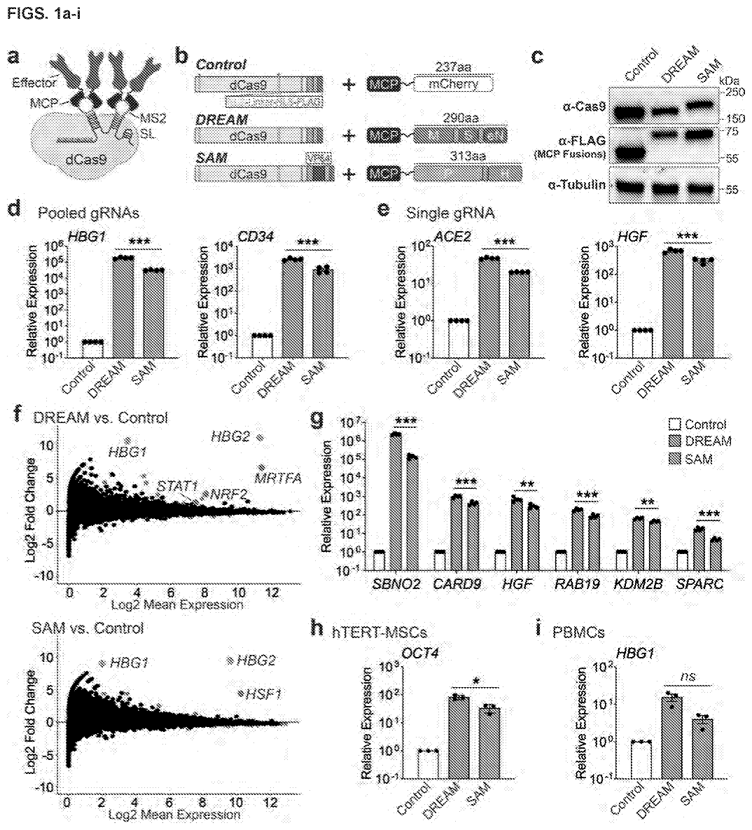

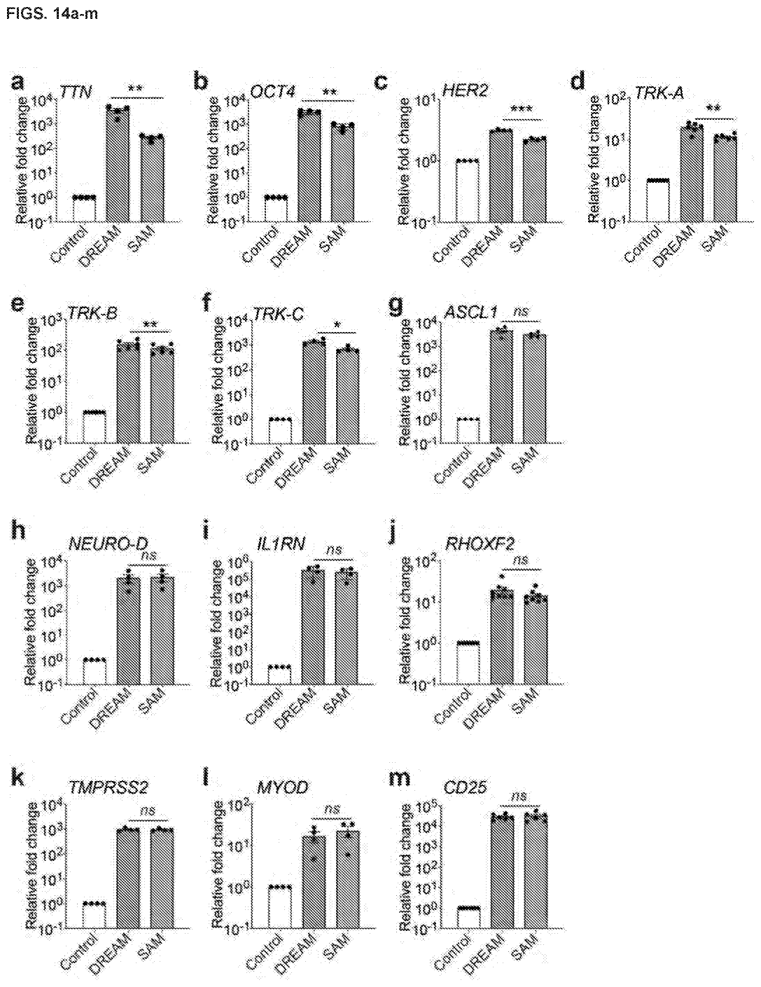

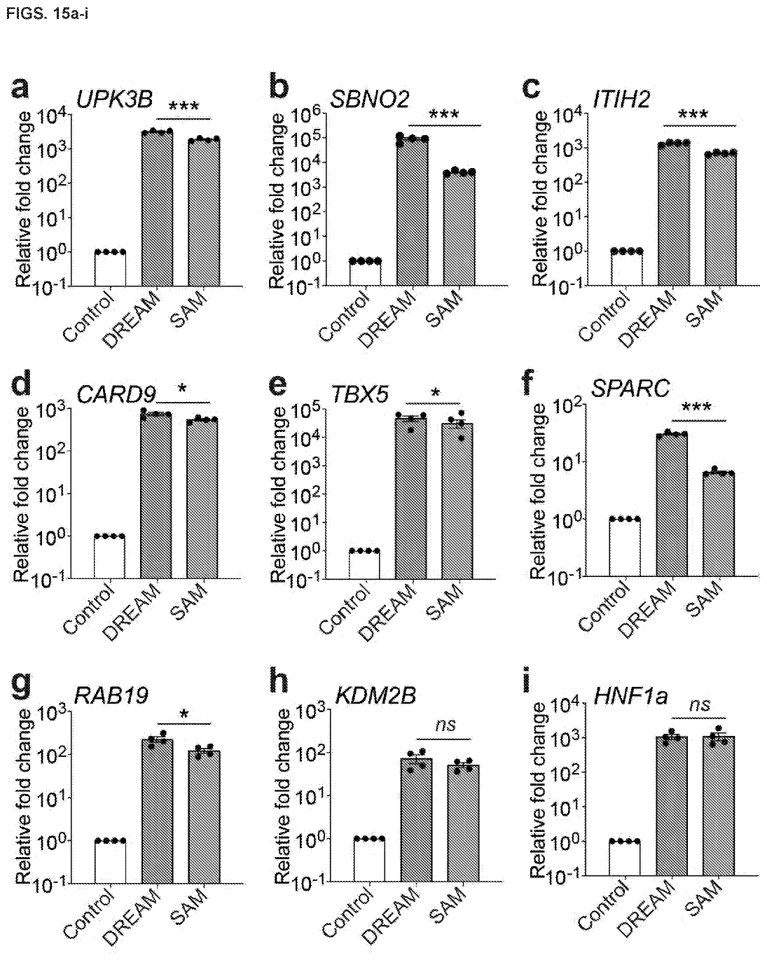

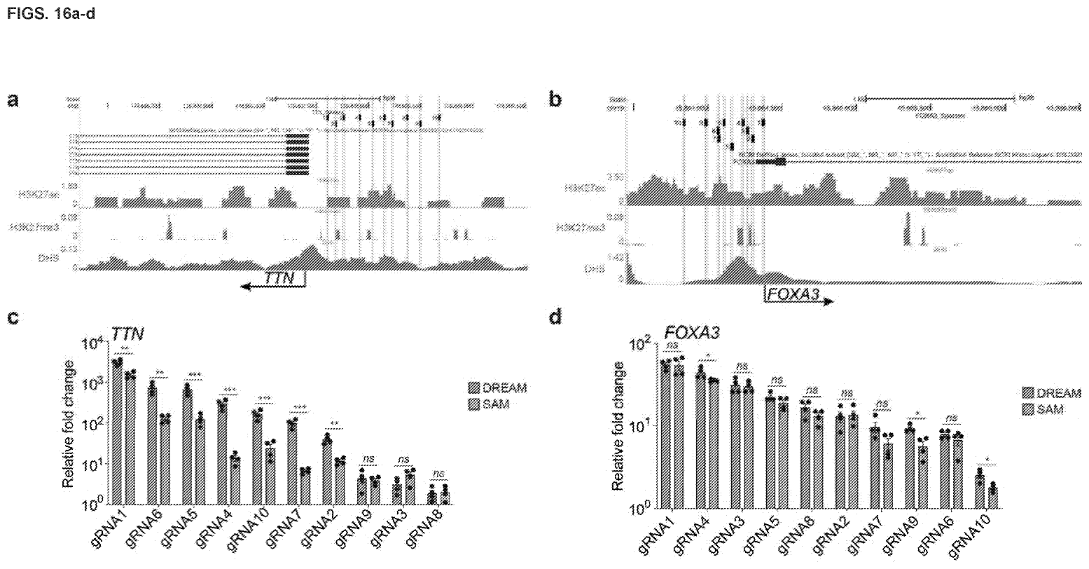

CRISPR-DREAM displays potent activation of endogenous promoters, is specific, and is robust across diverse mammalian cell types. To assess the relative transactivation potential of CRISPR-DREAM, the inventors first targeted the DREAM or SAM¹⁵ systems (FIGS. 1a-b), to different human promoters in HEK293T cells. All components for both the DREAM and SAM systems were well-expressed in HEK293T cells (FIG. 1c). At all promoters targeted using pools of gRNAs (n=15), DREAM was superior or comparable or to the SAM system (FIG. 1d and FIGS. 14a-m). Similarly, when human promoters were targeted using only single gRNAs (n=11), DREAM remained superior or comparable to the SAM system in all experiments (FIG. 1e and FIGS. 15a-i). Interestingly, this trend extended throughout ˜1 kb upstream of the transcription start sites (TSSs) surrounding human genes (FIGS. 16a-d). Collectively, these data demonstrate that, although the DREAM system is smaller than the SAM system, and is devoid of viral TADs, it displays superior or comparable transactivation potency in human cells.

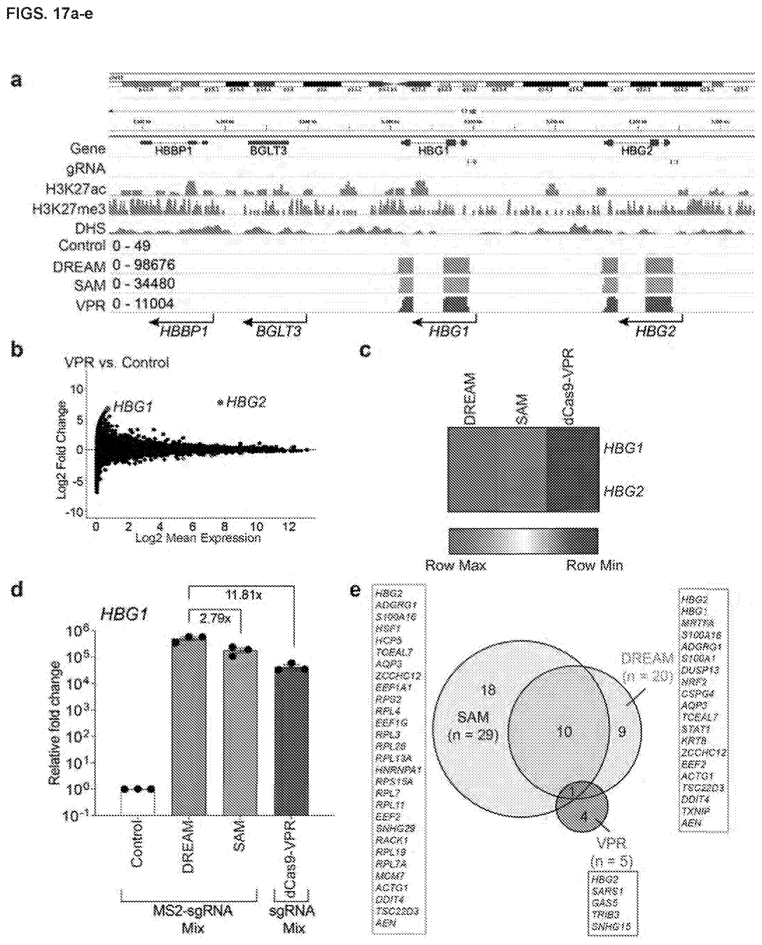

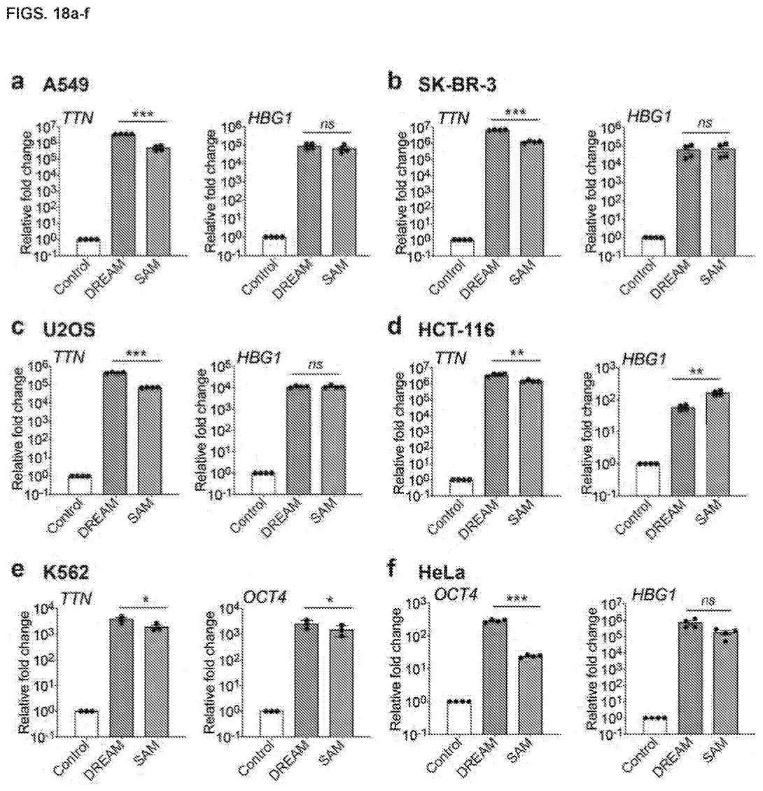

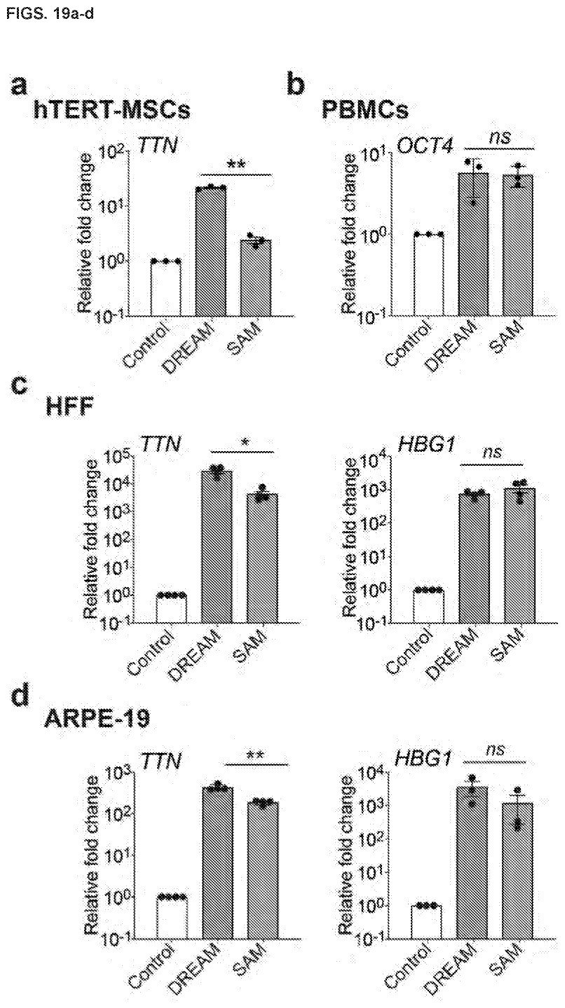

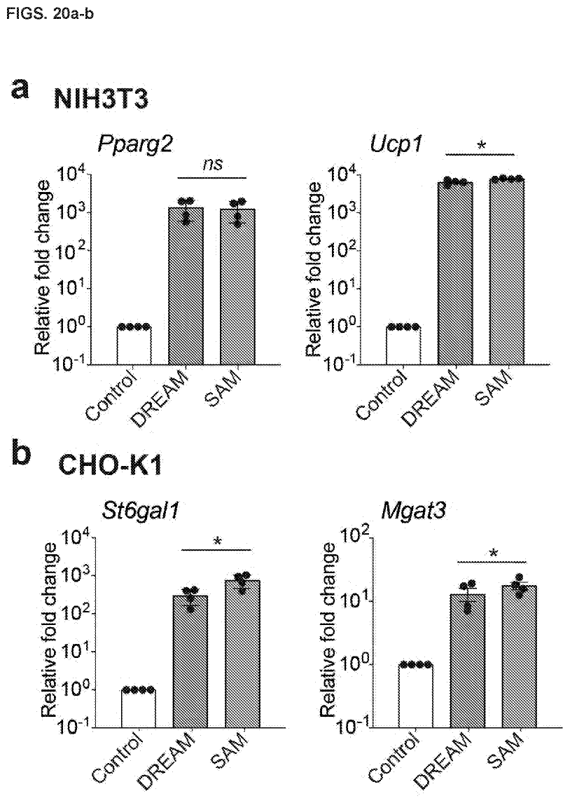

To test the transcriptome-wide specificity of CRISPR-DREAM, the inventors used 4 gRNAs to target the DREAM or the SAM system to the HBG1/HBG2 locus in HEK293T cells and then performed RNA-seq (FIG. 1f). HBG1/HBG2 gene activation was specific and potent for both the CRISPR-DREAM and SAM systems relative to dCas9+MCP-mCherry control treated cells. However, DREAM activated substantially more HBG1/HBG2 transcription than the SAM system or dCas9-VPR⁹ (FIG. 1f and FIGS. 17a-e). The inventors also found that the DREAM system was significantly (P<0.05) more potent than the SAM system at all targeted genes when each system was combined with a pool of six gRNAs, each targeting a different gene (FIG. 1g). Additionally, the inventors evaluated the efficacy of the DREAM system across a battery of different human cell types, including a diverse panel of cancer cell lines (FIG. 1h and FIGS. 18a-f) as well as primary and/or karyotypically normal human cells (FIG. 1i and FIGS. 19a-d). Finally, the inventors tested the transactivation potency of the DREAM system in mammalian cell types widely used for disease modeling/biocompatibility applications and therapeutic production pipelines (NIH3T3 and CHO-K1 cells, respectively; FIGS. 20a-b. Across all experiments the DREAM system displayed highly potent transactivation. Overall, these data demonstrate that CRISPR-DREAM is robust, broadly potent, specific, and functionally compatible with diverse human and mammalian cell types.

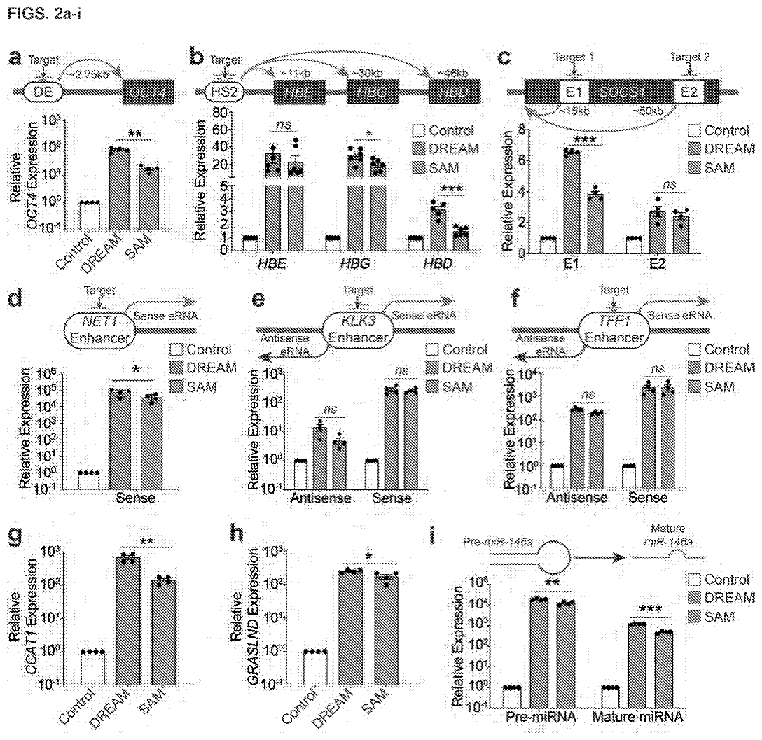

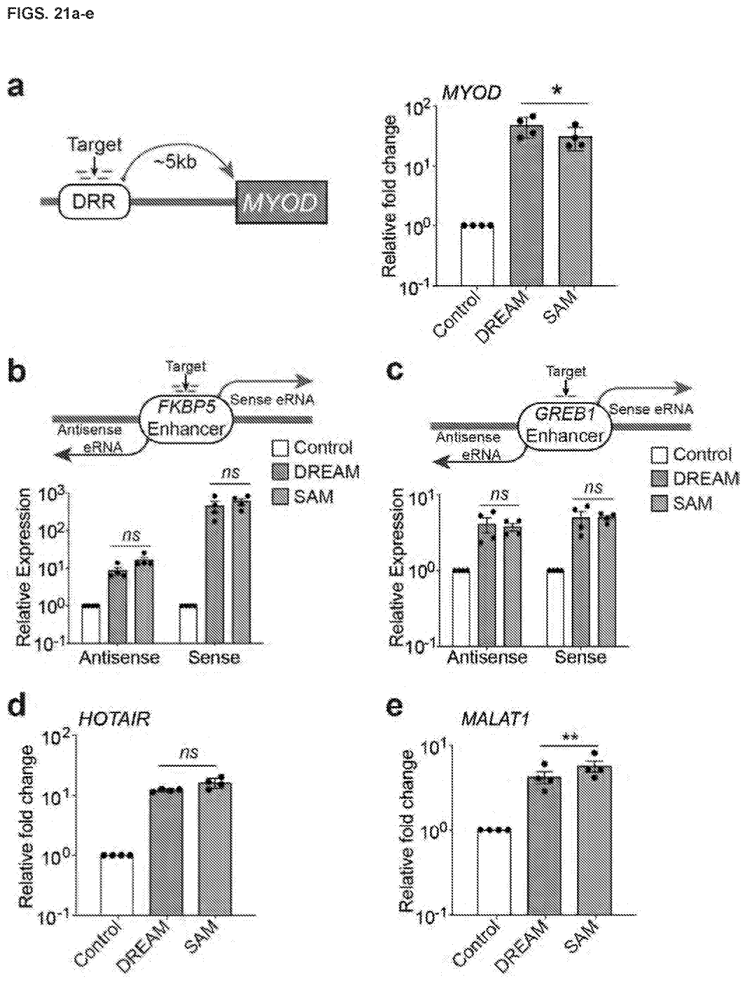

CRISPR-DREAM efficiently catalyzes RNA synthesis from noncoding genomic regulatory elements. Since CRISPR-DREAM efficiently and robustly activated mRNAs when targeted to promoter regions, the inventors next tested whether the DREAM system could also activate transcription from distal human regulatory elements (i.e., enhancers) and other non-coding transcripts (i.e., enhancer RNAs; eRNAs, long noncoding RNAs; lncRNAs, and microRNAs; miRNAs). The inventors first targeted the DREAM or SAM systems to the OCT4 distal enhancer (DE)⁴³ and found that the DREAM system significantly (P<0.05) upregulated OCT4 expression relative to the SAM system when targeted to the DE (FIG. 2a). Similar results were observed when targeting the DREAM system to the DRR enhancer⁴⁴ upstream of the MYOD gene (FIG. 21a). The inventors also targeted the DREAM system to the human HS2 enhancer^{45, 46} and observed that the DREAM system induced expression from the downstream HBE, HBG, and HBD genes (FIG. 2b). The inventors further observed transactivation of the SOCS1 gene when the DREAM system was targeted to either of two different intragenic SOCS1 enhancers; one located ˜15 kb, and the other ˜50 kb downstream of the SOCS1 TSS (FIG. 2c). Together these data demonstrate that CRISPR-DREAM can stimulate human gene expression when targeted to different classes of enhancers (those regulating a single-gene, multiple genes, or intragenic enhancers) embedded within native chromatin.

The inventors next tested whether CRISPR-DREAM could activate eRNAs when targeted to endogenous human enhancers. When targeted to the NET1 enhancer, the DREAM system activated eRNA transcription (FIG. 2d), consistent with other reports⁴⁷. Moreover, when the DREAM system was targeted to the bidirectionally transcribed KLK3 and TFF1 enhancers, the inventors observed substantial upregulation of eRNAs in both the sense and antisense directions (FIGS. 2e-f). Similar results were obtained when targeting the human FKBP5 and GREB1 enhancers (FIGS. 21b-c). CRISPR-DREAM also stimulated the production of endogenous lncRNAs when targeted to the CCAT1, GRASLND, HOTAIR, or MALAT1 loci (FIGS. 2g-h, FIGS. 21d-e). Finally, the inventors found that the DREAM system activated miRNA-146a expression when targeted to the miRNA-146a promoter (FIG. 2i). Taken together, these data show that CRISPR-DREAM can robustly transactivate regulatory regions spanning diverse classes of the human transcriptome.

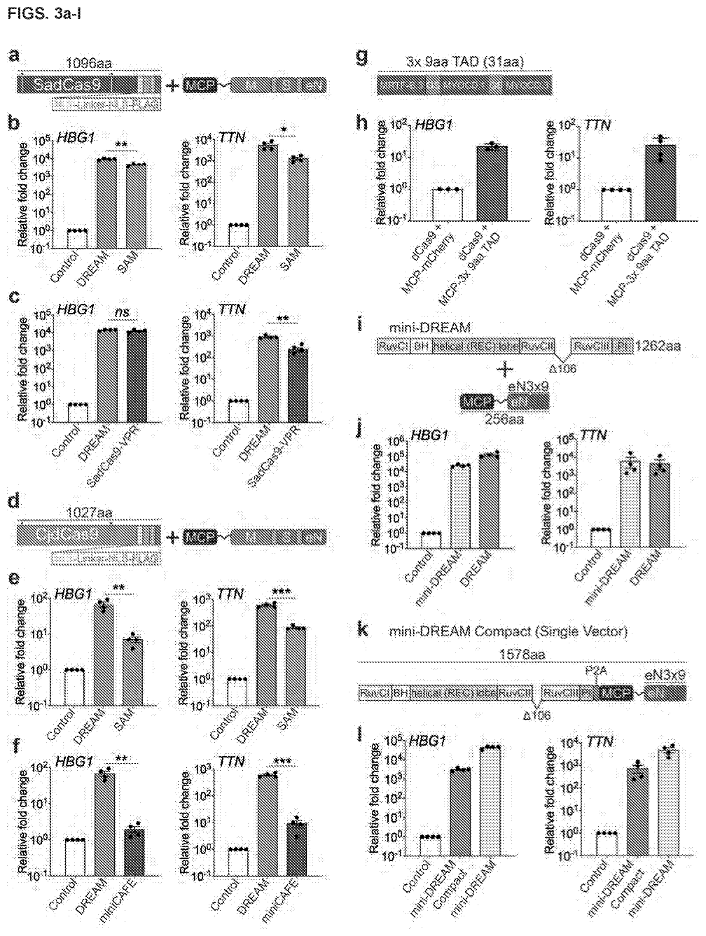

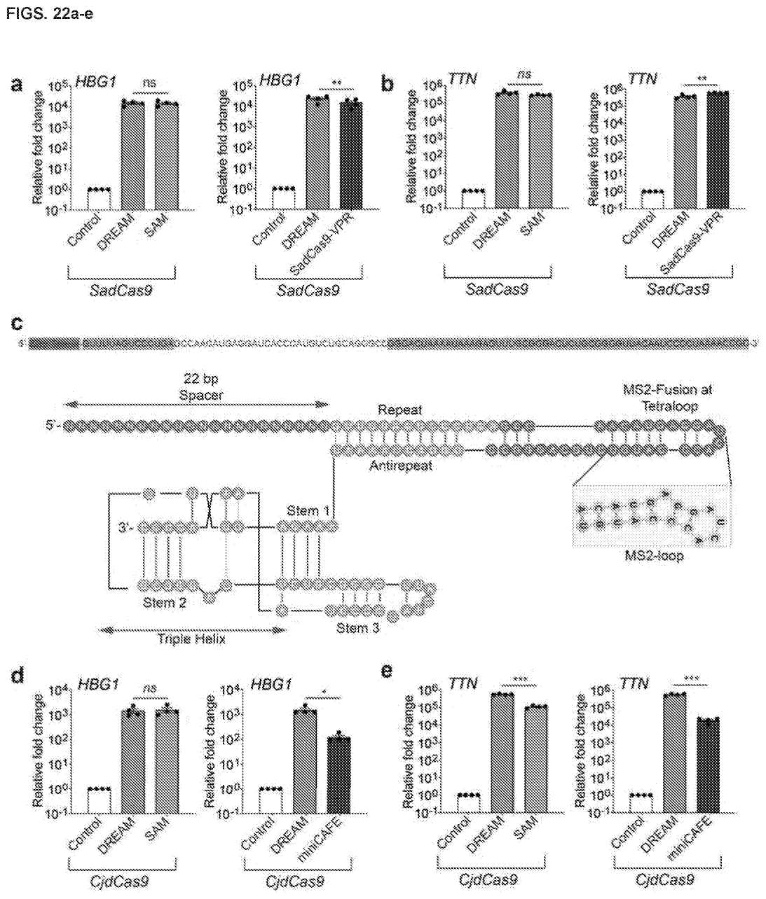

Smaller, orthogonal CRISPR-DREAM platforms enable expanded genomic targeting beyond NGG PAM sites. To enhance the versatility of CRISPR-DREAM beyond SpdCas9 and to expand targeting to non-NGG PAM sites, the inventors selected the two smallest naturally occurring orthogonal Cas9 proteins; SadCas9 (1,096aa) and CjdCas9 (1,027aa) for further analyses (FIG. 3a, FIG. 3d). The inventors used SaCas9-specific gRNAs harboring MS2 loops⁴⁸ to compare the potency between the SadCas9-DREAM and SAM systems in HEK293T cells. SadCas9-DREAM was significantly (P<0.05) more potent than SadCas9-SAM when targeted to either the HBG1 or TTN promoters (FIG. 3b). The inventors also found that SadCas9-DREAM outperformed or was comparable to SadCas9-VPR when targeted to these loci (FIG. 3c). CjdCas9-based transcriptional activation platforms have also recently been developed using viral TADs (miniCAFE)⁴⁹; however, gRNA-based recruitment of transcriptional modulators using CjdCas9 has not been described. Therefore, the inventors engineered the CjCas9 gRNA scaffold to incorporate an MS2 loop within the tetraloop of the CjCas9 gRNA scaffold (FIG. 22c). The inventors used this MS2-modified CjCas9 gRNA to generate CjdCas9-DREAM and compared the potency between CjdCas9-DREAM, CjdCas9-SAM, and the miniCAFE systems at the HBG1 or TTN promoters (FIGS. 3e-f) in HEK293T cells. At all targeted sites, CjdCas9-DREAM outperformed or was comparable to the CjdCas9-SAM or miniCAFE systems. The inventors also observed high levels of transactivation using SadCas9-DREAM and CjdCas9-DREAM in a different human cell line (FIGS. 22a, 22b, 22d, and 22e). These data demonstrate that DREAM is not only compatible with other orthogonal dCas9 targeting systems, but that it displays superior performance at most tested promoters.

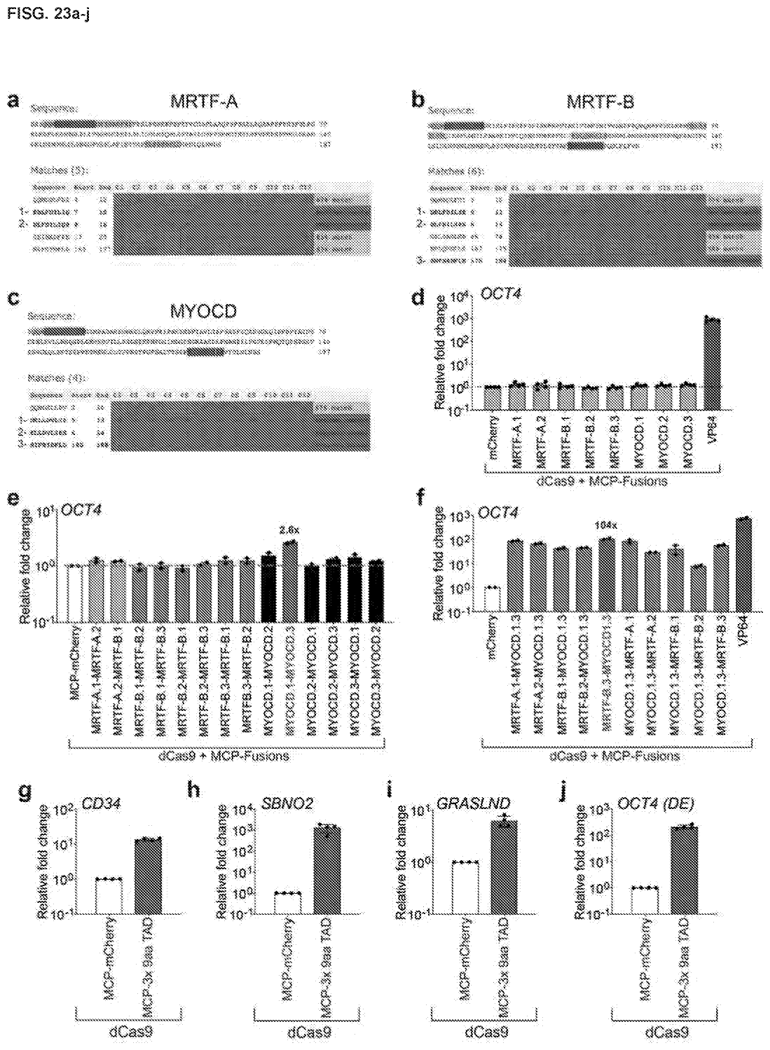

Generation and validation of a compact mini-DREAM system. The inventors next sought to reduce the sizes of the CRISPR-DREAM components. The inventors first investigated whether individual TADs could be minimized while still retaining the transactivation potency when recruited by dCas9. The inventors focused on individual TADs from MTFs that displayed transactivation potential (i.e., MRTF-A, MRTF-B, and MYCOD proteins, FIGS. 7a-g, FIG. 8a-f). 9aa TADs have been shown to synthetically activate transcription previously using GAL4 systems^{40, 50}. Therefore, the inventors used predictive software⁴⁰ to identify 9aa TADs in MRTF-A, MRTF-B, and MYCOD proteins, and recruited these TADs to human loci using dCas9 and MCP-MS2 fusions in single, bipartite, and tripartite formats (FIG. 23a-j). Interestingly, the inventors observed that only tripartite combinations of 9aa TADs were able to robustly activate endogenous gene expression, and to varying degrees (FIG. 23f). The inventors selected one tripartite 9aa combination (3× 9aa TAD; MRTF-B.3+MYOCD.1+MYOCD.3) for further analysis (FIG. 3g). This 3× 9aa TAD activated HBG1, TTN, and CD34 gene expression when recruited to corresponding promoters using dCas9 (FIG. 3h; FIG. 23g). The inventors also found that this 3× 9aa TAD combination could activate gene expression via a single gRNA, and moreover could transactivate other endogenous regulatory loci (FIGS. 23h-j). These results suggest that combinations of 9aa TADs can be used as minimal functional units to transactivate endogenous human loci when recruited via dCas9.

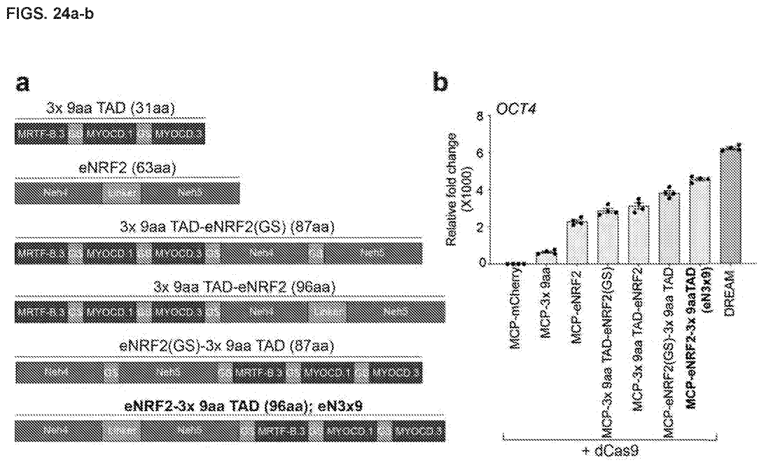

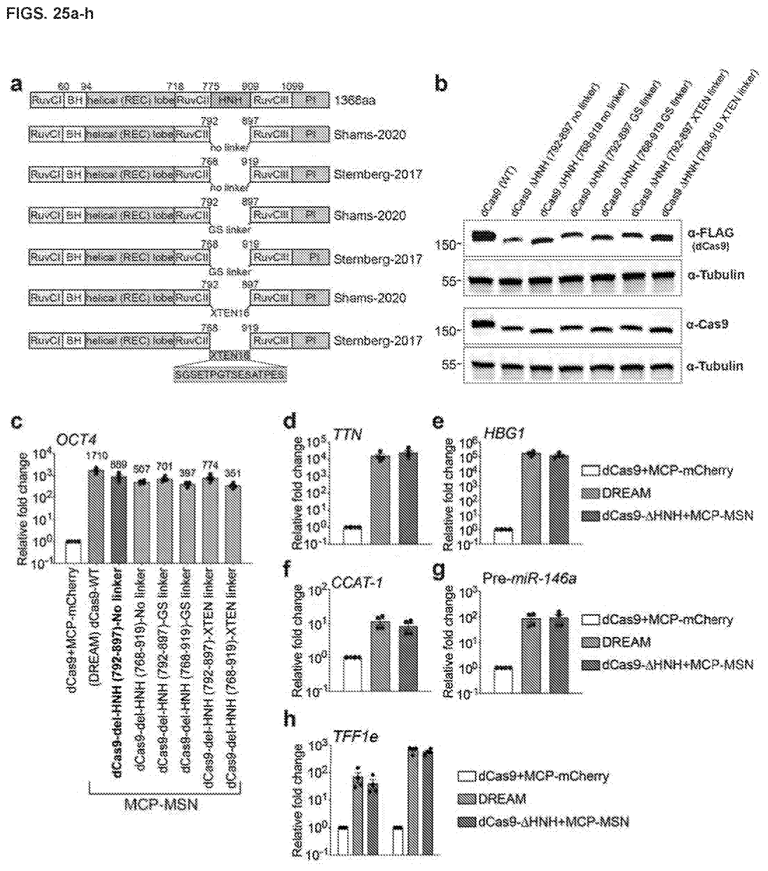

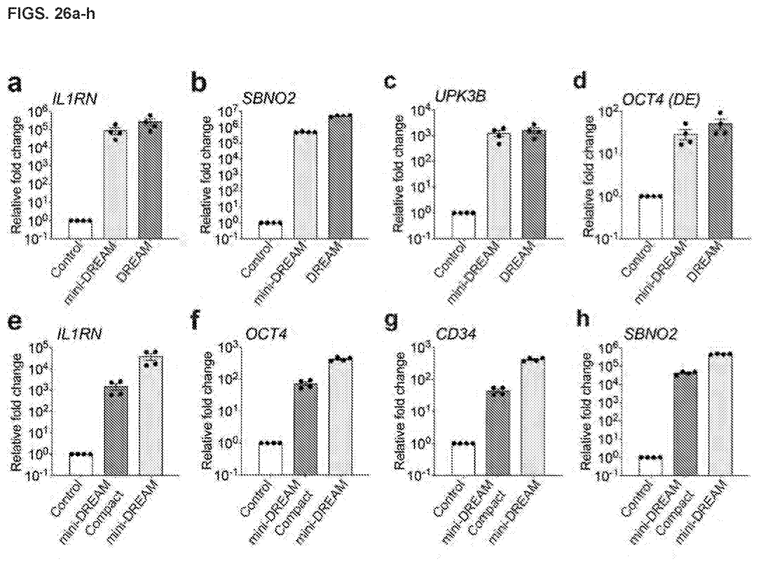

The inventors next combined the 3× 9aa TAD with the engineered NRF2 TAD (eNRF2) in four different combinations to generate a small, yet potent transactivation module called eN3×9 (FIGS. 24a-b). Notably, minimized Cas9 proteins that retain DNA binding activity have also been recently created^{51, 52}. Therefore, the inventors next evaluated the relative transactivation capabilities among a panel of minimized, HNH-deleted, dCas9 variants in tandem with MCP-MSN and found that an HNH-deleted variant without a linker between two RuvC domains was optimal, albeit with slight protein expression decreases (FIGS. 25a-b). The inventors further validated this linker-less, HNH-deleted CRISPR-DREAM variant at multiple human promoters and other regulatory elements (FIGS. 25c-h) and then combined this minimized dCas9 with MCP-eN3×9 to generate the mini-DREAM system (FIG. 3i). The mini-DREAM system transactivated HBG1, TTN, and IL1RN gene expression when recruited to corresponding promoters (FIG. 3j; FIG. 26a). The inventors also found that the mini-DREAM system could activate endogenous promoters via a single gRNA (FIGS. 26b-c), and could activate downstream gene expression when targeted to an upstream enhancer (FIG. 26d). Finally, the inventors evaluated whether the minimized components of the mini-DREAM system were functional when delivered within a single vector (FIG. 3k) and found that this compact, single vector mini-DREAM system retained transactivation potential when targeted to human promoters using pooled (FIG. 31; FIG. 26e-g), or a single gRNA (FIG. 26h). Overall, these data show that the components of the CRISPR DREAM system can be minimized to fit within a single vector delivery framework while retaining functionality.

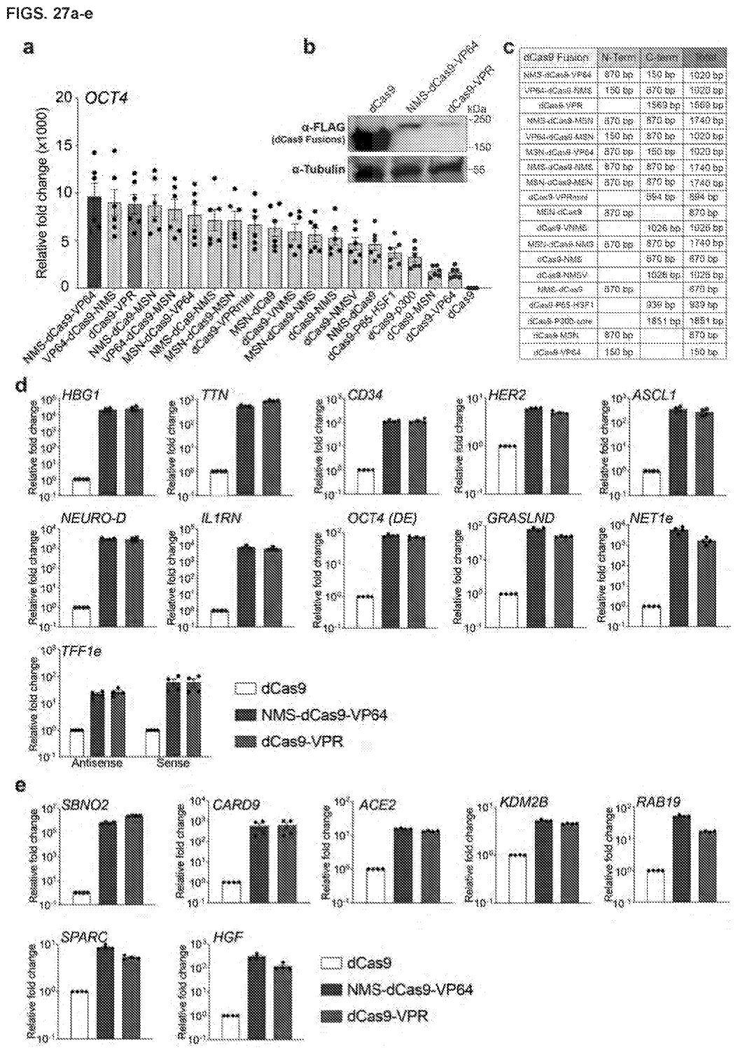

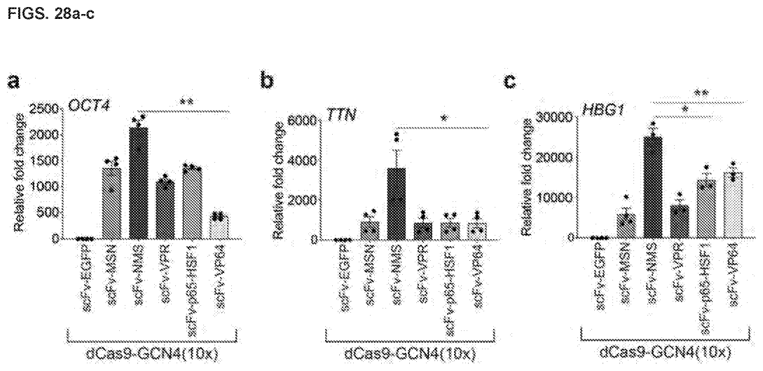

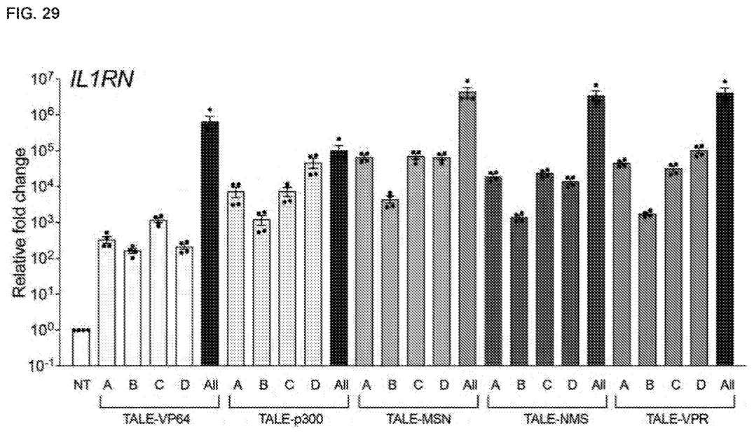

The MSN and NMS effector domains are robust across programmable DNA binding platforms. The inventors next tested the potency of tripartite MSN and NMS effectors when fused the to dCas9 in different architectures and observed that both effectors could activate gene expression when fused to the N- or C-terminus of dCas9 (FIGS. 27a-e) or when recruited via the Sun-Tag¹⁴ architecture (FIGS. 28a-c). Interestingly, in contrast to MCP-mediated recruitment (FIGS. 13a-c), additional TADs were observed to improve performance in direct fusion architectures (FIG. 27a, FIG. 27c). In the SunTag architecture, the NMS domain was superior to other benchmarked effector domains, such as VP64¹⁴, VPR⁵³, and p65-HSF1⁵⁴ (FIGS. 28a-c). To maximize the potential use of the MSN/NMS effector domains and explore their versatility, the inventors next tested whether each was capable of gene activation when fused to TALE or ZF scaffolds (FIG. 4a, FIG. 4d). Both effectors strongly transactivated IL1RN using a single TALE fusion protein (FIG. 29) or a pool of 4 TALE fusion proteins targeted to the IL1RN promoter (FIG. 4a). Similarly, both effectors activated ICAM1 expression using a single synthetic ZF fusion protein targeted to the ICAM1 promoter (FIG. 4b). These data demonstrate that the MSN and NMS effectors are compatible with diverse programmable DNA binding scaffolds beyond Type II CRISPR/Cas systems.

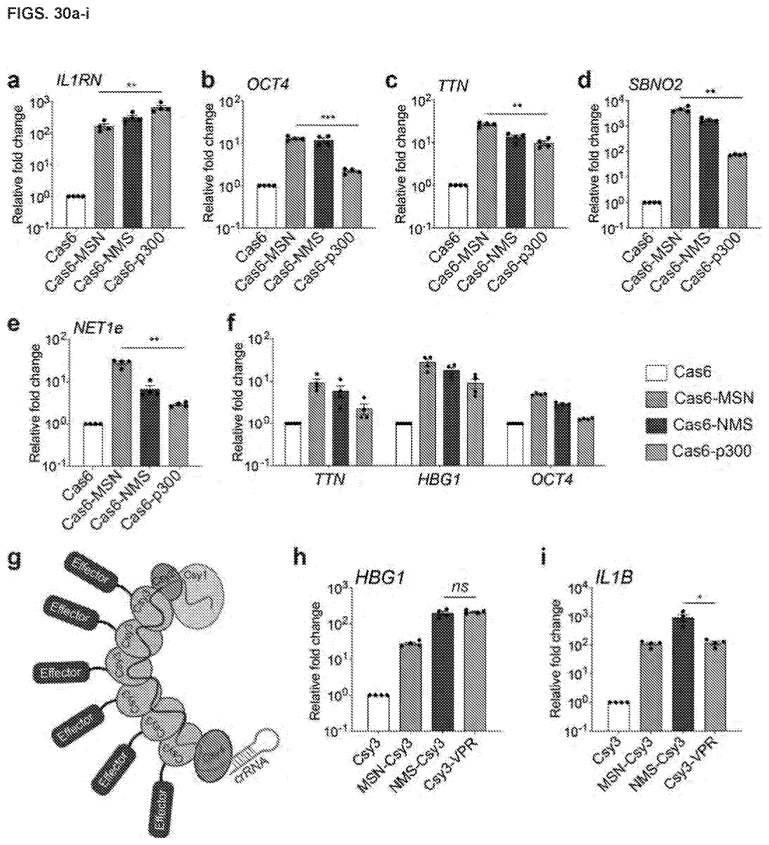

Transcriptional activators have recently been shown to modulate the expression of endogenous human loci when recruited by Type I CRISPR systems⁵⁵. Therefore, to evaluate whether MSN and/or NMS were functional beyond Type II CRISPR systems, the inventors fused each to the Cas6 component of the E. coli Type I CRISPR Cascade (Eco-Cascade) system (FIG. 4c). These data showed that Cas6-MSN (or NMS) performed comparably to the Cas6-p300 system when targeted to a spectrum of human promoters (FIG. 4d; Figd. 30a-d). The inventors also observed that the Cas6-MSN (or NMS) systems could activate eRNAs from when targeted to the endogenous NET1 enhancer (FIG. 30e). One advantage of CRISPR Cascade is that the system can process its own crRNA arrays, which can enable multiplexed targeting to the human genome. Previous reports have leveraged this capability to simultaneously activate two human genes⁵⁵. The inventors found that when Cas6 was fused to MSN, the CRISPR Cascade system could simultaneously activate up to six human genes when corresponding crRNAs were co-delivered in an arrayed format (FIG. 4e; FIG. 30f). The inventors also found that these transactivation capabilities were extensible to another Type I CRISPR system; Pae-Cascade⁵⁶ (FIGS. 30g-i). In sum, these data show that the MSN and NMS effectors are robust and directly compatible with programmable DNA binding platforms beyond Type II CRISPR systems without any additional engineering.

The NMS effector enables superior multiplexed gene activation when fused to dCas12a. The CRISPR/Cas12a system has attracted significant attention because the platform is smaller than SpCas9, and because Cas12a can process its own crRNA arrays in human cells⁵⁷. This feature has been leveraged for both multiplexed genome editing and multiplexed transcriptional control¹⁸. Therefore, the inventors next investigated the potency of the tripartite MSN and NMS effectors when they were directly fused to dCas12a (FIG. 4f). The inventors selected the AsdCas12a variant for this analysis because AsdCas12a (hereafter dCas12a) has been shown to activate human genes when fused to transcriptional effectors¹⁸. These results demonstrated that both dCas12a-MSN and dCas12a-NMS were able to induce gene expression when targeted to different human promoters using pooled or single crRNAs (FIG. 4g, FIG. 4h, FIGS. 31a-e). dCas12a-NMS was generally superior to dCas12a-MSN and to the previously described dCas12a-Activ system¹⁸ at the loci tested here. These data demonstrate that the NMS and MSN effectors domains are potent transactivation modules when combined with the dCas12a targeting system in human cells.

The inventors next tested the extent to which dCas12a-MSN/NMS could be used in conjunction with crRNA arrays for multiplexed endogenous gene activation. The inventors cloned 8 previously described crRNAs¹⁸ (targeting the ASCL1, ILIR2, IL1B or ZFP42 promoters) into a single plasmid in an array format and then transfected this vector into HEK293T cells with either dCas12a control, dCas12a-MSN, dCas12a-NMS, or the dCas12a-Activ system. Again, these data demonstrated that dCas12a-NMS was superior or comparable to dCas12a-Activ, even in multiplex settings (FIG. 31f). Finally, to evaluate if dCas12-NMS could simultaneously activate multiple genes on a larger scale, the inventors cloned 20 full-length (20 bp) crRNAs targeting 16 different loci into a single array (FIG. 31g). This array was designed to enable simultaneous targeting of several classes of human regulatory elements; including 13 different promoters, 2 different enhancers (one intrageneric; SOCS1, and one driving eRNA output; NET1), and one lncRNA (GRASLND). When this crRNA array was transfected into HEK293T cells along with dCas12a-NMS, RNA synthesis was robustly stimulated from all 16 loci (FIG. 4i). To the inventors' knowledge this is the most loci that have been targeted simultaneously using CRISPR systems, demonstrating the versatility and utility of the engineered NMS effector in combination with dCas12a. dCas9-NMS permits efficient reprogramming of human fibroblasts in vitro.

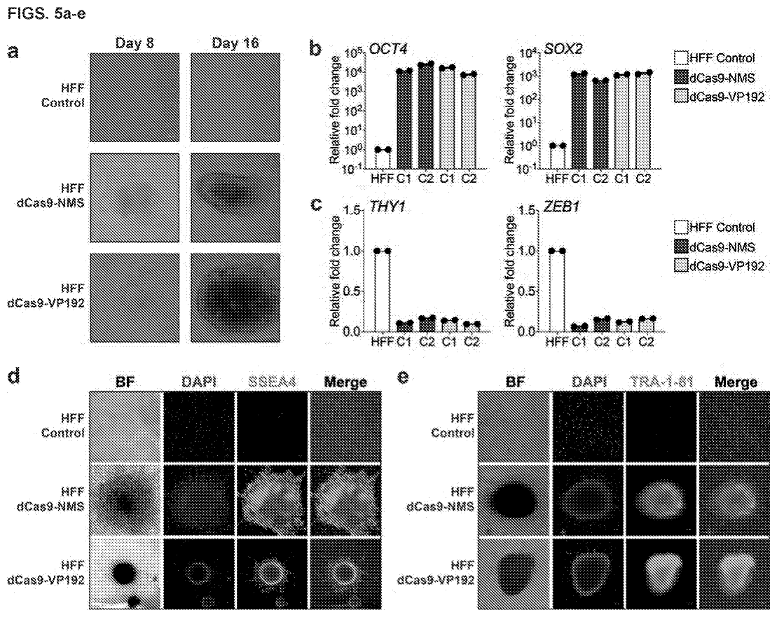

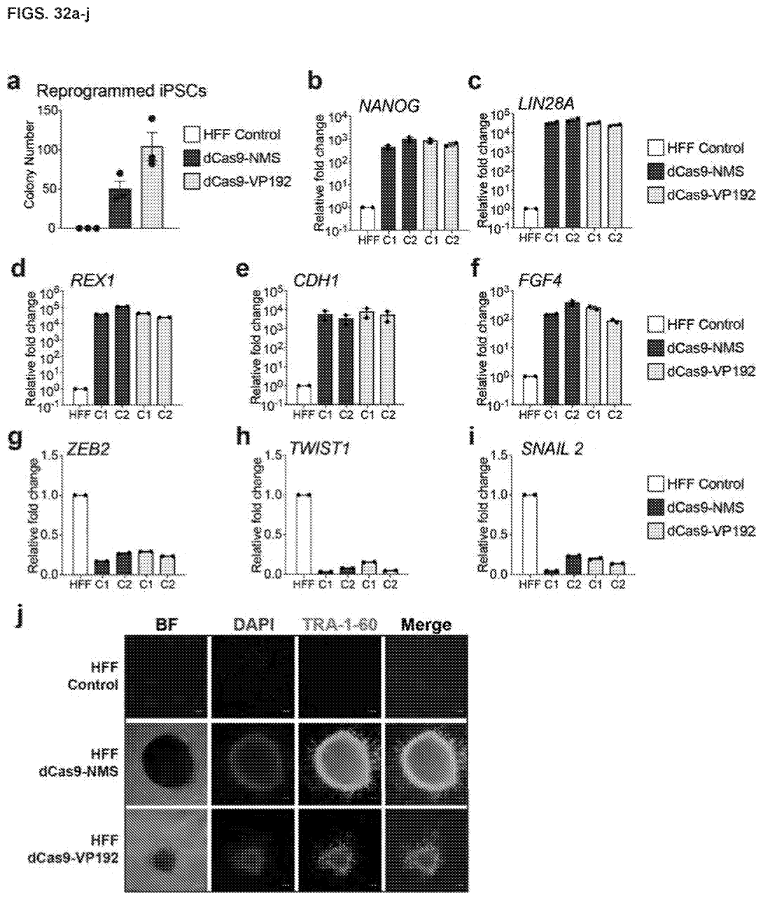

CRISPRa systems using repeated portions of the alpha herpesvirus VP16 TAD (dCas9-VP192) have been used to efficiently reprogram human foreskin fibroblasts (HFFs) into induced pluripotent stem cells (iPSCs)¹⁷. To evaluate the functional capabilities of the inventors' engineered human transactivation modules, the inventors fused the NMS domain directly to the C-terminus of dCas9 (dCas9-NMS) and tested its ability to reprogram HFFs. The inventors used a direct dCas9 fusion architecture so that the inventors could leverage gRNAs previously optimized for this reprogramming strategy and to better compare dCas9-NMS with the corresponding state of the art (dCas9-VP192)¹⁷. The inventors used the NMS effector as opposed to MSN, as NMS displayed more potency than MSN when directly fused to dCas9 (FIG. 27a). The inventors targeted dCas9-NMS (or dCas9-VP192) to endogenous loci using the 15 gRNAs previously optimized to reprogram HFFs to pluripotency with the dCas9-VP192 system. Using this approach, the inventors observed morphological changes beginning by 8 days post-nucleofection (FIG. 5a) and efficient reprogramming by 16 days post-nucleofection, although to a lesser extent than when using dCas9-VP192 (FIG. 32a).

The inventors picked and expanded iPSC colonies and then measured the expression of pluripotency and mesenchymal genes ˜40 days post-nucleofection. The inventors found that genes typically associated with pluripotency (OCT4, SOX2, NANOG, LIN28A, REX1, CDH1, and FGF4)^{58, 59} were highly expressed in colonies derived from HFFs nucleofected with the gRNA cocktail and dCas9-NMS or dCas-VP192 (FIG. 5b; FIGS. 32b-f). Conversely, the inventors observed that genes typically associated with fibroblast/mesenchymal cell identity (THY1, ZEB1, ZEB2, TWIST, and SNAIL2)^{58, 59} were poorly expressed in colonies derived from HFFs nucleofected with the gRNA cocktail and dCas9-NMS or dCas-VP192 (FIG. 5c; FIGS. 32g-i). Finally, the inventors assessed the expression of pluripotency associated markers (SSEA-4, TRA-1-81 and TRA-1-60)⁶⁰ and found that all were highly expressed in iPSC colonies derived from HFFs nucleofected with the gRNA cocktail and either dCas9-NMS or dCas-VP192 (FIG. 5d, FIG. 5e; FIG. 32j). These data show that engineered transactivation modules sourced from human MTFs can be used to efficiently reprogram complex cell phenotypes, including cell lineage.

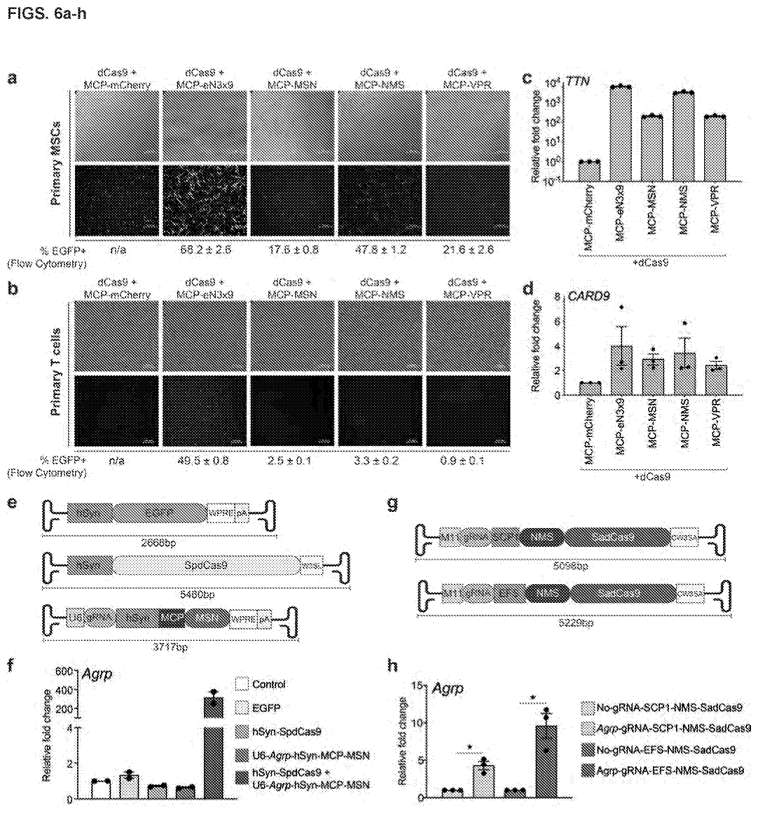

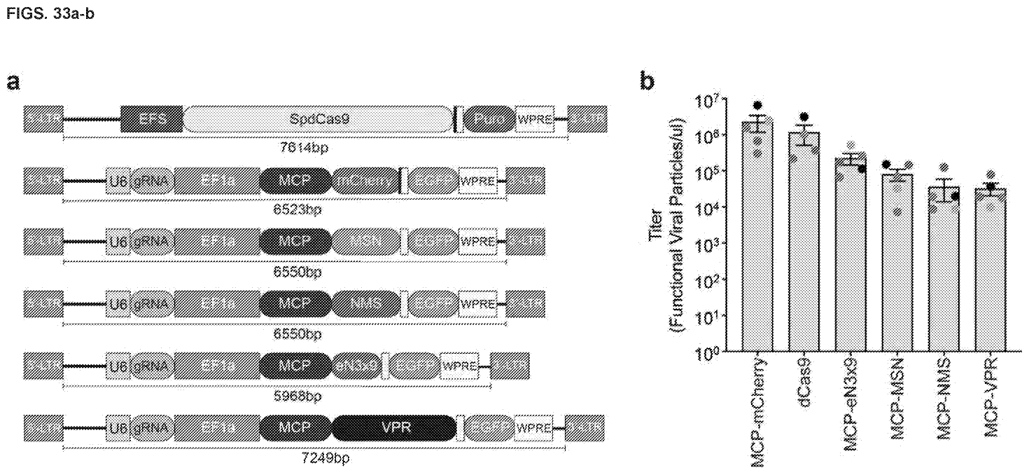

The MSN, NMS, and eN3×9 transactivation modules are well tolerated and effective in clinically useful primary human cell types. The recent development of CRISPRa tools has enabled new therapeutic opportunities^{6, 61}. However, it has been shown that in some cases, CRISPRa tools harboring viral TADs can be poorly tolerated, and even toxic^{12, 62-64} This prompted us to test the relative expression and efficacy of the human MTF derived multipartite TADs MSN, NMS, and eN3×9 tools in comparison to the viral multipartite TAD VPR in therapeutically relevant human primary cells. The inventors selected primary human umbilical cord MSCs and primary T cells for analysis. Lentiviral transduction was selected to ensure high levels of payload delivery. Interestingly, the inventors observed that lentiviral titers were influenced by fused TAD, with MCP fused to eN3×9 consistently generating the highest titers (FIGS. 33a-b). The inventors next transduced MSCs using an MOI of ˜10.0 for all conditions and observed variable expression levels among MCP fusions proteins at 72 hours post transduction using both microscopy and flow cytometry (FIG. 6a) despite using equal amounts of lentivirus. For instance, although MCP-eN3×9 and MCP-NMS displayed high levels expression via microscopy, MCP-VPR and MCP-MSN were relatively poorly expressed. Similarly, the inventors also tested the expression levels of these MCP fusions in primary T cells using lentiviral transduction at an MOI of ˜5.0 and observed that MCP-eN3×9 displayed the highest expression levels 72 hours post transduction, while MCP-VPR showed the lowest expression (FIG. 6b).

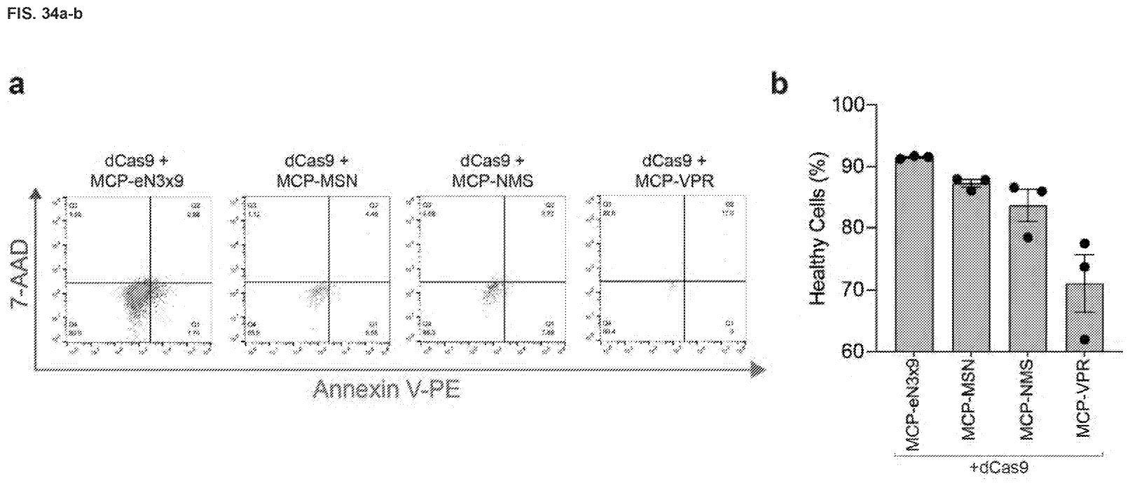

The inventors next assessed the gene activation capabilities of these MCP-TAD fusions in primary MSCs and T cells. In MSCs, eN3×9 outperformed all other effectors, and VPR showed the lowest potency when targeted to the TTN promoter (FIG. 6c). In primary T cells each TAD activated CARD9 expression to relatively similar and modest levels when targeted to the CARD9 promoter (FIG. 6d). However, in primary T cells the inventors observed that the human MTF derived multipartite TADs resulted in dramatically better T cell viability than the viral multipartite TAD VPR (FIGS. 34a-b). Collectively these data demonstrate that the human MTF derived multipartite MSN, NMS, and eN3×9 TADs are as or more potent than the VPR TAD, while also maintaining similar or superior expression levels in therapeutically relevant human primary cells. Notably, MSN, NMS, and eN3×9 are also much smaller than the VPR TAD, and in the case of primary T cells, are also much less cytotoxic.

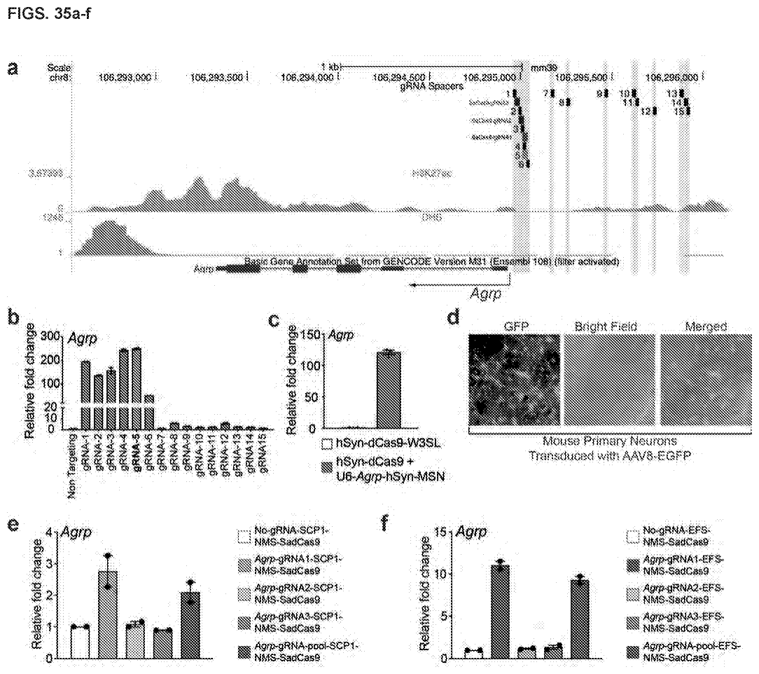

Dual and all-in-one AAV mediated delivery of CRISPR-DREAM and SadCas9-NMS systems efficiently activates gene expression in primary neurons. AAV mediated delivery has emerged as a powerful method to deliver therapeutic payloads in vitro⁶⁵ and in vivo⁶⁶. However, due to strict payload limitations, the delivery of CRISPRa tools using AAV has been limited to dual AAV systems and/or the use of viral TADs^{67, 68}. To assess the transcriptional activation potential of the compact CRISPR-DREAM components in combination with AAV mediated delivery, the inventors targeted the murine Agrp gene, which modulates food intake behavior and obesity^{69, 70}, as a proof of concept. The inventors first tested 15 individual gRNAs targeting a ˜1 kb window upstream of the Agrp promoter in Neuro-2a cells to identify a top performing gRNA (FIGS. 35a-b). Based on this result, the inventors constructed a dual AAV delivery system, wherein one AAV expressed dCas9, and the other AAV expressed the top performing Agrp-targeting gRNA along with MCP-MSN (FIG. 6e). Both recombinant AAVs (and an EGFP control AAV) used the AAV8 serotype capsid to ensure efficient neuronal transduction⁷¹ (FIG. 35e). In dual AAV-transduced (dCas9 and gRNA/MCP-MSN, respectively) primary murine neurons, the inventors observed high levels of Agrp activation (FIG. 6f).

Encouraged by this result using a dual AAV strategy, the inventors next designed two different all-in-one (AIO) AAV approaches (FIG. 6g). These designs leveraged the M11 promoter to express a gRNA, and either the SCP1 or EFS promoter to drive the expression of NMS fused to the N-terminus of SadCas9. NMS was prioritized over MSN as it showed higher potency when fused to N-terminus of dCas9 (FIGS. 28a-c). To further reduce packaging size, the inventors also selected compact engineered WPRE and PolyA³⁸ tail elements in these construct designs. After selecting a top performing Agrp-targeting SadCas9 gRNA in Neuro-2A cells (FIGS. 35g-h), the inventors made recombinant AAVs (using serotype AAV8) and delivered these AIO AAVs to primary murine neurons. In both cases, the inventors observed significant (P value <0.05) transcriptional upregulation of Agrp, with the EFS promoter harboring vector displaying superiority to the SCP promoter harboring vector (FIG. 6h). These data demonstrate that the compact components of the CRISPR-DREAM retain high transactivation potency when delivered using either dual or AIO AAV modalities.

### Example 3—Discussion

Here, the inventors harnessed the programmability and versatility of different dCas9-based recruitment architectures (direct fusion, gRNA-aptamer, and SunTag-based) to optimize the transcriptional output of TADs derived from natural human TFs. The inventors leveraged these insights to build superior and widely applicable transactivation modules that are portable across all modern synthetic DNA binding platforms, and that can activate the expression of diverse classes endogenous RNAs. The inventors selected mechanosensitive TFs (MTFs) for biomolecular building blocks because they naturally display rapid and potent gene activation at target loci, can interact with diverse transcriptional co-factors across different human cell types, and because their corresponding TADs are relatively small⁷²⁻⁷⁴. The inventors not only identified and validated the transactivation potential of TADs sourced from individual MTFs, but the inventors also established the optimal TAD sequence compositions and combinations for use across different synthetic DNA binding platforms, including Type I, II and V CRISPR systems, TALE proteins, and ZF proteins.

Our study also revealed that for MTFs, tripartite fusions using TADs from MRTA-A (M), STAT1 (S), and NRF2 (N) in one of two different combinations (either MSN or NMS) consistently resulted in the most potent human gene activation across different DNA binding platforms. Interestingly, each of these components has been shown to interact with key transcriptional co-factors. For example, individual TADs from MRTF-A, STAT1, NRF2 can directly interact with endogenous p300^{29, 75}. Moreover, the Neh4 and Neh5 TADs from NRF2 can also cooperatively recruit endogenous CBP for transcriptional activity^{27, 76}. Therefore, the inventors suspect that the potency of the MSN and NMS tripartite effector proteins is likely related to their robust capacity to recruit the powerful and ubiquitous endogenous transcriptional modulators p300 and/or CBP, which is likely positively impacted by their direct tripartite fusion.

Additionally, this study demonstrated that the superior transactivation capabilities of the CRISPR/dCas9-recruited enhanced activation module (DREAM) system—consisting of dCas9 and a gRNA-aptamer recruited MCP-MSN fusion—are not reliant upon the direct fusion(s) of any other proteins (viral or otherwise) to dCas9, in contrast to the SAM system which relies upon dCas9-VP64¹⁵. The inventors used this advantage to combine the MCP-MSN module with HNH domain deleted dCas9 variants^{51, 52}, which exhibited similar potencies to full-size dCas9 variants. To further reduce the size of CRISPR-DREAM, the inventors built a minimal transactivation module (eN3×9; 96aa) by evaluating the potency of a suite of 9aa TADs from MTFs and by next combining the most potent variants with the small eNRF2 TAD. The inventors then combined the minimized eN3×9 transactivation module with an HNH domain deleted dCas9 variant in two-vector (mini-DREAM) and single-vector (mini-DREAM compact) delivery architectures, which retained potent transactivation capabilities.

The inventors also integrated the MSN and NMS effectors with the Type I CRISPR/Cascade and Type II dCas12a platforms to enable superior multiplexed endogenous activation of human genes. This multiplexing capability holds tremendous promise for reshaping endogenous cellular pathways and/or engineering complex transcriptional networks. dCas9-based transcription factors harboring viral TADs have also been used for directed differentiation and cellular reprogramming^{9, 17, 77, 78}. Here, the inventors showed that the inventors could reprogram human fibroblasts into iPSCs using dCas9 directly fused to the NMS transcriptional effector with similar gene expression profiles, times to conversion, and morphological characteristics compared to iPSCs derived using dCas9 fused to viral TADs¹⁷. However, dCas9-NMS resulted in slightly fewer iPSC colonies than dCas9-VP192, which the inventors attribute to the reprogramming framework tested here being optimized for use with dCas9-VP192.

The inventors also demonstrated that the MSN and NMS effectors were compatible with dual and all-in-one (AIO) AAV vectors. Additionally, the AIO AAV vector design, which combines the short SCP1 promoter, the short M11 gRNA promoter and the compact CW3SA modified WPRE/poly A tail elements, holds tremendous potential for future delivery architectures. Similarly, the potency of AIO AAV vectors encoding NMS-SadCas9 empower researchers with a new streamlined modality to induce endogenous gene expression in vivo that could be used within animal models or clinical settings. Finally, the inventors found that the NMS, MSN, and eN3×9 TADs were well-expressed and potent in therapeutically important human cells. Although the tripartite VPR TAD contains the potent VP64 and RTA viral elements, in the inventors' primary cell experiments VPR showed the lowest expression levels and gene activation potencies. In contrast, the hypercompact eN3×9 TAD was well expressed in both MSCs and T cells. In MSCs eN3×9 was also extremely potent, however in T cells, gene activation efficacy was modest for all activators tested. Nevertheless, MSN, NMS, and eN3×9 TADs were substantially less toxic compared to the VPR TAD in T cells. Further analyses at other target sites and over longer time courses will likely be useful for optimized therapeutic use cases.

In summary, the inventors have used the rational redesign of natural human TADs to build synthetic transactivation modules that enable consistent and potent performance across programmable DNA binding platforms, mammalian cell types, and genomic regulatory loci embedded within human chromatin. Although the inventors used MTFs as sources of TADs here, the inventors' work establishes a framework that could be used with practically any natural or engineered TF and/or chromatin modifier in future efforts. The potency, small size, versatility, capacity for multiplexing, and the lack of viral components associated with the newly engineered MSN, NMS, and eN3×9 TADs and CRISPR-DREAM systems developed here could be valuable tools for fundamental and biomedical applications requiring potent and predictable activation of endogenous eukaryotic transcription.

### ****************

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references, to the extent that they provide exemplary procedural or other details supplementary to those set forth herein, are specifically incorporated herein by reference.
1. Qi, L. S. et al. Repurposing CRISPR as an RNA-guided platform for sequence-specific control of gene expression. Cell 152, 1173-1183 (2013).
2. Gilbert, L. A. et al. CRISPR-mediated modular RNA-guided regulation of transcription in eukaryotes. Cell 154, 442-451 (2013).
3. Perez-Pinera, P. et al. RNA-guided gene activation by CRISPR-Cas9-based transcription factors. Nat Methods 10, 973-976 (2013).
4. Thakore, P. I., Black, J. B., Hilton, I. B. & Gersbach, C. A. Editing the epigenome: technologies for programmable transcription and epigenetic modulation. Nat Methods 13, 127-137 (2016).
5. Liao, H. K. et al. In Vivo Target Gene Activation via CRISPR/Cas9-Mediated Trans-epigenetic Modulation. Cell 171, 1495-1507 e1415 (2017).
6. Goell, J. H. & Hilton, I. B. CRISPR/Cas-Based Epigenome Editing: Advances, Applications, and Clinical Utility. Trends Biotechnol 39, 678-691 (2021).
7. Gemberling, M. P. et al. Transgenic mice for in vivo epigenome editing with CRISPR-based systems. Nat Methods 18, 965-974 (2021).
8. Cabrera, A. et al. The sound of silence: Transgene silencing in mammalian cell engineering. Cell Syst 13, 950-973 (2022).
9 Chavez, A. et al. Highly efficient Cas9-mediated transcriptional programming. Nat Methods 12, 326-328 (2015).
10. Hilton, I. B. et al. Epigenome editing by a CRISPR-Cas9-based acetyltransferase activates genes from promoters and enhancers. Nat Biotechnol 33, 510-517 (2015).
11. Li, J. et al. Programmable human histone phosphorylation and gene activation using a CRISPR/Cas9-based chromatin kinase. Nat Commun 12, 896 (2021).
12. Wang, K. et al. Systematic comparison of CRISPR-based transcriptional activators uncovers gene-regulatory features of enhancer-promoter interactions. Nucleic Acids Res (2022).
13. Escobar, M. et al. Quantification of Genome Editing and Transcriptional Control Capabilities Reveals Hierarchies among Diverse CRISPR/Cas Systems in Human Cells. ACS Synth Biol 11, 3239-3250 (2022).
14. Tanenbaum, M. E., Gilbert, L. A., Qi, L. S., Weissman, J. S. & Vale, R. D. A protein-tagging system for signal amplification in gene expression and fluorescence imaging. Cell 159, 635-646 (2014).
15. Konermann, S. et al. Genome-scale transcriptional activation by an engineered CRISPR-Cas9 complex. Nature 517, 583-588 (2015).
16. Zalatan, J. G. et al. Engineering complex synthetic transcriptional programs with CRISPR RNA scaffolds. Cell 160, 339-350 (2015).
17. Weltner, J. et al. Human pluripotent reprogramming with CRISPR activators. Nat Commun 9, 2643 (2018).
18. Campa, C. C., Weisbach, N. R., Santinha, A. J., Incarnato, D. & Platt, R. J. Multiplexed genome engineering by Cas12a and CRISPR arrays encoded on single transcripts. Nat Methods 16, 887-893 (2019).
19. Li, K. et al. Interrogation of enhancer function by enhancer-targeting CRISPR epigenetic editing. Nat Commun 11, 485 (2020).
20. Dominguez, A. A. et al. CRISPR-Mediated Synergistic Epigenetic and Transcriptional Control. CRISPR J 5, 264-275 (2022).
21. Lambert, S. A. et al. The Human Transcription Factors. Cell 175, 598-599 (2018).
22. Soto, L. F. et al. Compendium of human transcription factor effector domains. Mol Cell (2021).
23. Tycko, J. et al. High-Throughput Discovery and Characterization of Human Transcriptional Effectors. Cell 183, 2020-2035 e2016 (2020).
24. Alerasool, N., Leng, H., Lin, Z. Y., Gingras, A. C. & Taipale, M. Identification and functional characterization of transcriptional activators in human cells. Mol Cell 82, 677-695 e677 (2022).
25. Mammoto, A., Mammoto, T. & Ingber, D. E. Mechanosensitive mechanisms in transcriptional regulation. J Cell Sci 125, 3061-3073 (2012).
26. Wagh, K. et al. Mechanical Regulation of Transcription: Recent Advances. Trends Cell Biol 31, 457-472 (2021).
27. Katoh, Y. et al. Two domains of Nrf2 cooperatively bind CBP, a CREB binding protein, and synergistically activate transcription. Genes Cells 6, 857-868 (2001).
28 Galli, G. G. et al. YAP Drives Growth by Controlling Transcriptional Pause Release from Dynamic Enhancers. Mol Cell 60, 328-337 (2015).
29. He, H. et al. Transcriptional factors p300 and MRTF-A synergistically enhance the expression of migration-related genes in MCF-7 breast cancer cells. Biochem Biophys Res Commun 467, 813-820 (2015).
30. Zanconato, F. et al. Transcriptional addiction in cancer cells is mediated by YAP/TAZ through BRD4. Nat Med 24, 1599-1610 (2018).
31. Dasgupta, I. & McCollum, D. Control of cellular responses to mechanical cues through YAP/TAZ regulation. J Biol Chem 294, 17693-17706 (2019).
32. Zhao, J. et al. Chemokines protect vascular smooth muscle cells from cell death induced by cyclic mechanical stretch. Sci Rep 7, 16128 (2017).
33. McSweeney, S. R., Warabi, E. & Siow, R. C. Nrf2 as an Endothelial Mechanosensitive Transcription Factor: Going With the Flow. Hypertension 67, 20-29 (2016).
34. Schmidt, R. et al. CRISPR activation and interference screens decode stimulation responses in primary human T cells. Science 375, eabj4008 (2022).
35. Weltner, J. & Trokovic, R. Reprogramming of Fibroblasts to Human iPSCs by CRISPR Activators. Methods Mol Biol 2239, 175-198 (2021).
36. Gao, Z. et al. Engineered miniature H1 promoters with dedicated RNA polymerase II or III activity. J Biol Chem 296, 100026 (2021).
37. Juven-Gershon, T., Cheng, S. & Kadonaga, J. T. Rational design of a super core promoter that enhances gene expression. Nat Methods 3, 917-922 (2006).
38. Choi, J. H. et al. Optimization of AAV expression cassettes to improve packaging capacity and transgene expression in neurons. Mol Brain 7, 17 (2014).
39. Barde, I., Salmon, P. & Trono, D. Production and titration of lentiviral vectors. Curr Protoc Neurosci Chapter 4, Unit 4 21 (2010).
40. Piskacek, S. et al. Nine-amino-acid transactivation domain: establishment and prediction utilities. Genomics 89, 756-768 (2007).
41. Zhao, B. et al. Inactivation of YAP oncoprotein by the Hippo pathway is involved in cell contact inhibition and tissue growth control. Genes Dev 21, 2747-2761 (2007).
42. Bromberg, J. & Darnell, J. E., Jr. The role of STATs in transcriptional control and their impact on cellular function. Oncogene 19, 2468-2473 (2000).
43. Nordhoff, V. et al. Comparative analysis of human, bovine, and murine Oct-4 upstream promoter sequences. Mamm Genome 12, 309-317 (2001).
44. Chen, J. C., Love, C. M. & Goldhamer, D. J. Two upstream enhancers collaborate to regulate the spatial patterning and timing of MyoD transcription during mouse development. Dev Dyn 221, 274-288 (2001).
45. Tolhuis, B., Palstra, R. J., Splinter, E., Grosveld, F. & de Laat, W. Looping and interaction between hypersensitive sites in the active beta-globin locus. Mol Cell 10, 1453-1465 (2002).
46. Carter, D., Chakalova, L., Osborne, C. S., Dai, Y. F. & Fraser, P. Long-range chromatin regulatory interactions in vivo. Nat Genet 32, 623-626 (2002).
47. Zhang, Z. et al. Transcriptional landscape and clinical utility of enhancer RNAs for eRNA-targeted therapy in cancer. Nat Commun 10, 4562 (2019).
48. Nishimasu, H. et al. Crystal Structure of Staphylococcus aureus Cas9. Cell 162, 1113-1126 (2015).
49. Zhang, X. et al. MiniCAFE, a CRISPR/Cas9-based compact and potent transcriptional activator, elicits gene expression in vivo. Nucleic Acids Res 49, 4171-4185 (2021).
50. Piskacek, M., Vasku, A., Hajek, R. & Knight, A. Shared structural features of the 9aaTAD family in complex with CBP. Mol Biosyst 11, 844-851 (2015).
51. Sternberg, S. H., LaFrance, B., Kaplan, M. & Doudna, J. A. Conformational control of DNA target cleavage by CRISPR-Cas9. Nature 527, 110-113 (2015).
52. Shams, A. et al. Comprehensive deletion landscape of CRISPR-Cas9 identifies minimal RNA-guided DNA-binding modules. Nat Commun 12, 5664 (2021).
53. Kunii, A. et al. Three-Component Repurposed Technology for Enhanced Expression: Highly Accumulable Transcriptional Activators via Branched Tag Arrays. CRISPR J 1, 337-347 (2018).
54. Zhou, H. et al. In vivo simultaneous transcriptional activation of multiple genes in the brain using CRISPR-dCas9-activator transgenic mice. Nat Neurosci 21, 440-446 (2018).
55. Pickar-Oliver, A. et al. Targeted transcriptional modulation with type I CRISPR-Cas systems in human cells. Nat Biotechnol 37, 1493-1501 (2019).
56. Chen, Y. et al. Repurposing type I-F CRISPR-Cas system as a transcriptional activation tool in human cells. Nat Commun 11, 3136 (2020).
57. Zetsche, B. et al. Multiplex gene editing by CRISPR-Cpf1 using a single crRNA array. Nat Biotechnol 35, 31-34 (2017).
58. Polo, J. M. et al. A molecular roadmap of reprogramming somatic cells into iPS cells. Cell 151, 1617-1632 (2012).
59. Nishimura, K. et al. Manipulation of KLF4 expression generates iPSCs paused at successive stages of reprogramming. Stem Cell Reports 3, 915-929 (2014).
60. Takahashi, K. et al. Induction of pluripotent stem cells from adult human fibroblasts by defined factors. Cell 131, 861-872 (2007).
61. Bashor, C. J., Hilton, I. B., Bandukwala, H., Smith, D. M. & Veiseh, O. Engineering the next generation of cell-based therapeutics. Nat Rev Drug Discov 21, 655-675 (2022).
62. Weuring, W. J. et al. CRISPRa-Mediated Upregulation of scn1laa During Early Development Causes Epileptiform Activity and dCas9-Associated Toxicity. CRISPR J 4, 575-582 (2021).
63. Ewen-Campen, B. et al. Optimized strategy for in vivo Cas9-activation in Drosophila. Proc Natl Acad Sci USA 114, 9409-9414 (2017).
64. Yamagata, T. et al. CRISPR/dCas9-based Scn1a gene activation in inhibitory neurons ameliorates epileptic and behavioral phenotypes of Dravet syndrome model mice. Neurobiol Dis 141, 104954 (2020).
65. Royo, N. C. et al. Specific AAV serotypes stably transduce primary hippocampal and cortical cultures with high efficiency and low toxicity. Brain Res 1190, 15-22 (2008).
66. George, L. A. et al. Multiyear Factor VIII Expression after AAV Gene Transfer for Hemophilia A. N Engl J Med 385, 1961-1973 (2021).
67. Matharu, N. et al. CRISPR-mediated activation of a promoter or enhancer rescues obesity caused by haploinsufficiency. Science 363 (2019).
68. Kemaladewi, D. U. et al. A mutation-independent approach for muscular dystrophy via upregulation of a modifier gene. Nature 572, 125-130 (2019).
69. Wallentin, L. et al. Efficacy and safety of dabigatran compared with warfarin at different levels of international normalised ratio control for stroke prevention in atrial fibrillation: an analysis of the RE-LY trial. Lancet 376, 975-983 (2010).
70. Beutler, L. R. et al. Obesity causes selective and long-lasting desensitization of AgRP neurons to dietary fat. Elife 9 (2020).
71. Pignataro, D. et al. Adeno-Associated Viral Vectors Serotype 8 for Cell-Specific Delivery of Therapeutic Genes in the Central Nervous System. Front Neuroanat 11, 2 (2017).
72. Ramana, C. V., Chatterjee-Kishore, M., Nguyen, H. & Stark, G. R. Complex roles of Stat1 in regulating gene expression. Oncogene 19, 2619-2627 (2000).
73. Esnault, C. et al. Rho-actin signaling to the MRTF coactivators dominates the immediate transcriptional response to serum in fibroblasts. Genes Dev 28, 943-958 (2014).
74. Tonelli, C., Chio, I. I. C. & Tuveson, D. A. Transcriptional Regulation by Nrf2. Antioxid Redox Signal 29, 1727-1745 (2018).
75. Wojciak, J. M., Martinez-Yamout, M. A., Dyson, H. J. & Wright, P. E. Structural basis for recruitment of CBP/p300 coactivators by STAT1 and STAT2 transactivation domains. EMBO J 28, 948-958 (2009).
76. Sun, Z., Chin, Y. E. & Zhang, D. D. Acetylation of Nrf2 by p300/CBP augments promoter-specific DNA binding of Nrf2 during the antioxidant response. Mol Cell Biol 29, 2658-2672 (2009).
77. Black, J. B. et al. Master Regulators and Cofactors of Human Neuronal Cell Fate Specification Identified by CRISPR Gene Activation Screens. Cell Rep 33, 108460 (2020).
78. Liu, Y. et al. CRISPR Activation Screens Systematically Identify Factors that Drive Neuronal Fate and Reprogramming. Cell Stem Cell 23, 758-771 e758 (2018).

1. A recombinant transcription activator comprising transcription activation domains MRTF-A, STAT1 and eNRF2.

2. The recombinant transcription activator of claim 1, further comprising a genomic regulatory element targeting domain and/or RNA-binding protein.

3. The recombinant transcription activator of claim 2, wherein said genomic regulatory element targeting domain is a Cas protein, such as Cas6, AsdCas12a, SpdCas9, CjdCas9, or SadCas9.

4. The recombinant transcription activator of claim 2, wherein said genomic regulatory element targeting domain is a TALE DNA binding domain or a zinc finger DNA binding domain.

5. The recombinant transcription activator of claim 1, wherein the transcription activation domains are ordered MRTF-A, STAT1 and eNRF2 in an N- to C-terminal order.

6. The recombinant transcription activator of claim 1, wherein the transcription activation domains are ordered eNRF2, MRTF-A and STAT1 in an N- to C-terminal order.

7. The recombinant transcription activator of claim 2, wherein said transcription activation domains are directly linked to said genomic regulatory element targeting domain.

8. The recombinant transcription activator of claim 2, wherein said transcription activation domains are linked to said genomic regulatory element targeting domain through a linking moiety.

9. The recombinant transcription activator of claim 8, wherein the linking moiety is GS or XTEN.

10. The recombinant transcription activator of claim 1, wherein the recombinant transcription activator is about 250-500 or about 290 amino acid residues in length.

11. A recombinant nucleic acid segment encoding a transcription activator comprising transcription activation domains MRTF-A, STAT1 and eNRF2.

12. The recombinant nucleic acid segment of claim 11, further comprising a nucleic acid segment encoding a genomic regulatory element targeting domain and/or RNA-binding protein.

13. The recombinant nucleic acid segment of claim 12, wherein said genomic regulatory element targeting domain is a Cas protein, such as Cas6, AsdCas12a, SpdCas9, CjdCas9, or SadCas9.

14. The recombinant nucleic acid segment of claim 12, wherein said genomic regulatory element targeting domain is a TALE DNA binding domain or a zinc finger DNA binding domain.

15. The recombinant nucleic acid segment of claim 11, wherein the transcription activation domain coding regions are ordered MRTF-A, STAT1 and eNRF2 in an N- to C-terminal order.

16. The recombinant nucleic acid segment of claim 11, wherein the transcription activation domain coding regions are ordered MRTF-A and STAT1 and eNRF2 or eNRF2, MRTF-A and STAT1 in an N- to C-terminal order.

17. The recombinant nucleic acid segment of claim 12, wherein said transcription activation domain coding regions are directly linked to said genomic regulatory element targeting domain coding region.

18. The recombinant nucleic acid segment of claim 12, wherein said transcription activation domain coding regions are linked to said genomic regulatory element targeting domain coding region through a coding region for a linking moiety.

19. The recombinant nucleic acid segment of claim 18, wherein the linking moiety is GS and/or XTEN.

20. The recombinant nucleic acid segment of claim 11, wherein the recombinant nucleic acid segment is about 750-1500 bp or about 870 bp in length.

21. The recombinant nucleic acid segment of claim 11, wherein the promoter is active in eukaryotic cell such as EFS or CMV.

22. An artificial recombinant transcription factor comprising or consisting of at least 3 repeated 9aa TADs generated from MRTF-B and MYOCD or transcription factors.

23. The artificial recombinant transcription factor of claim 22, wherein said recombinant transcription factor is about 250-500 or about 290 amino acids in size.

24. The artificial recombinant transcription factor of claim 22, wherein MRTF-B and MYOCD linked by linking moiety.

25. The artificial recombinant transcription factor of claim 24, further comprising the linking moieties GS and/or XTEN.

26. A method of editing gene expression in a eukaryotic cell comprising transferring into said cell the recombinant nucleic acid segment of claim 11.

27. The method of claim 26, wherein the gene regulatory element targeting domain is a Cas protein, and the method further comprises providing to said eukaryotic cell a guide RNA.

28. The method of claim 26, wherein said eukaryotic cell is an isolated cell in culture.

29. The method of claim 26, wherein said eukaryotic cell is derived from a living organism.

30. The method of claim 26, wherein said eukaryotic cell is a human cell or non-human mammalian cell.

31. The method of claim 26, wherein said eukaryotic cell is a fibroblast.

32. The method of claim 26, wherein editing results in one or more of (a) increased gene expression of one or multiple genes, (b) induction of cellular differentiation, (c) induction of cellular de-differentiation.

33. The method of claim 32, wherein editing results in induction of pluripotency/stem cells from a differentiated cell.

34. The method of claim 32, wherein editing results in expression of a native/endogenous gene in a cell deficient in expression of said native gene/endogenous gene.

35. The method of claim 32, wherein editing results in expression of a non-native/exogenous gene such that said cell is protected from or at reduced risk of development of a disease state, disease condition or disorder.

36. The method of claim 32, wherein editing system is delivered via a viral mechanism, such as adeno-associated virus, lentivirus, retrovirus, herpesvirus, baculovirus, or adenovirus.

37. The method of claim 32, wherein editing system is delivered via a non-viral mechanism, such as electroporation, nucleofection, mechanical stress, or liposomal transfer.


SEQUENCE LISTING (7 sequences)

SEQ ID NO: 1 - protein, 9 aa, synthetic construct
QQQXXXQQQ

SEQ ID NO: 2 - protein, 9 aa, synthetic construct
PPPXXXPPP

SEQ ID NO: 3 - protein, 6 aa, synthetic construct
IIXXII

SEQ ID NO: 4 - protein, 34 aa, synthetic construct
LTPEQVVAIASHDGGKQALETVQRLLPVLCQAHG

SEQ ID NO: 5 - protein, 6 aa, synthetic construct
GSSGSS

SEQ ID NO: 6 - protein, 7 aa, synthetic construct
GSSSSSS

SEQ ID NO: 7 - protein, 15 aa, synthetic construct
GGGGSGGGGSGGGGS